(19) United States
(12) Patent Application Publication
(10) Pub. No.: US 2018/0311508 A1
(43) Pub. Date: Nov. 1, 2018

(54) SYSTEM FOR OPTOGENETIC THERAPY

(71) Applicant: Circuit Therapeutics, Inc., Menlo Park, CA (US)

(72) Inventors: Brian Andrew Ellingwood, Sunnyvale, CA (US); Brian Beckey, Redwood City, CA (US); Ethan Berry, Redwood City, CA (US); Bruce Modesitt, Petaluma, CA (US); Dan Andersen, Menlo Park, CA (US); David Angeley, Charlottesville, VA (US); Michael Drews, Palo Alto, CA (US); T.D. Barbara Nguyen-Vu, Menlo Park, CA (US); Laura Leung, South San Francisco, CA (US); Andrei T. Popescu, Albany, CA (US)

(73) Assignee: Circuit Therapeutics, Inc., Menlo Park, CA (US)

(21) Appl. No.: 15/911,070

(22) Filed: Mar. 2, 2018

Related U.S. Application Data
(60) Provisional application No. 62/466,311, filed on Mar. 2, 2017.

(51) Int. Cl. A61N 5/06; A61M 25/00

(52) U.S. Cl. CPC A61N 5/0622; A61N 5/0601; A61N 2005/067; A61M 25/00; A61N 2005/063; A61N 5/062

(57) ABSTRACT

One embodment is directed to a probe for illuminating a target tissue of a patient, comprising: a plurality of optical fibers; a probe body portion having proximal and distal ends, the probe body portion being moveably coupled to the plurality of fibers and configured to at least partially encapsulate the plurality of fibers; a distal end portion coupled to the distal end of the probe body portion, the distal end portion comprising at least one guiding feature configured to redirect a path of at least one of the optical fibers comprising the plurality of optical fibers as such at least portion of one of the optical fibers is extended through and past the distal end portion by moving the plurality of fibers relative to the probe body portion. The probe further may comprise an ejector portion configured to move the plurality of fibers relative to the probe body portion.

### RELATED APPLICATION DATA

The present application claims priority to U.S. Provisional Application Ser. No. 62/466,311, filed Mar. 2, 2017. The foregoing application is hereby incorporated by reference into the present application in its entirety.

### FIELD OF THE INVENTION

This invention relates to systems and methods for treating neurological and other disorders, and more specifically to optogenetic dosage configurations, distributed emission devices, multi-fiber connectors, and anchoring configurations for use therewith

### BACKGROUND

Optogenetics utilizes light responsive membrane transport proteins to provide a means to selectively and controllably alter the function of cells. Determination of the safe ranges of such parameters, particularly in neural tissue, are among those central to the development of optogenetics as a direct therapy, with the ultimate goal of providing alteration in the targeted tissue function that results in safe and effective disease or symptom treatment. Furthermore, if use of the technique as a research tool, particularly when contemplated on a long term basis, such parameters are of importance to ensure research results are from excitation of the cell through light delivery to the opsin, rather than some overall collective impact of the light on the health of the cell.

For example, optogenetic technologies and techniques have been utilized in laboratory settings to change the membrane voltage potentials of excitable cells, such as neurons, and to study the behavior of such neurons before and after exposure to light of various wavelengths. In neurons, membrane depolarization leads to the activation of transient electrical signals (also called action potentials or “spikes”), which are the basis of neuronal communication. Conversely, membrane hyperpolarization leads to the inhibition of such signals. By exogenously expressing light-activated proteins that change the membrane potential in neurons, light can be utilized as a triggering means to induce inhibition or excitation. Thus, optogenetic therapies generally involve delivery of a light-sensitive membrane transport protein to a cell, which will then promote flux of specific ions across a cell membrane in response to specific wavelengths of light.

One example is channelrhodopsin (ChR) which is a light sensitive cation channel which, in response to blue light, opens and permits flow of sodium (Na+) ions across the cell membrane. In neurons, this causes depolarization and activation of the neuron containing this channel. An alternative example is halorhodopsin (NpHR, derived from the halobacterium Natronomonas pharaonis), a light-sensitive anion pump which pumps chloride (Cl−) ions into a cell in response to yellow light. When the cell is a neuron, NpHR will hyperpolarize the cell, thereby inhibiting it. In the context of optogenetic application, NpHR acts as an electrogenic chloride pump to increase the separation of charge across the plasma membrane of the targeted cell upon activation by yellow light. NpHR is a true pump and requires constant light to move through its photocycle. Since 2007, a number of modifications to NpHR have been made to improve its function. Codon-optimization of the DNA sequence followed by enhancement of its subcellular trafficking (eNpHR2.0 and eNpHR3.0) resulted in improved membrane targeting and higher currents more suitable for use in mammalian tissue. In addition, proton pumps archaerhodopsin-3 (“Arch”) and “eARCH”, and ArchT, Leptosphaeria maculans fungal opsins (“Mac”), enhanced bacteriorhodopsin (“eBR”), and Guillardia theta rhodopsin-3 (“GtR3”) have been developed as optogenetic tools. As described in further detail below, these optogenetic proteins, when activated by light, may be used to hyperpolarize the targeted cells by pumping hydrogen ions out of such cells. A new class of channel, recently described by Karl Deisseroth et al, such as in Science, April 2014. 344(6182):420-4, and Jonas Weitek, et al, in Science, April 2014. 344(6182):409-12, each of which are incorporated by reference in their entirety, that is based on ChR but is modified to permit cations to pass through the “inhibitory” channel (which may be termed, by way of non-limiting examples; “iChR”, “iC1C2”, “ChloC”, or “SwiChR”) will open and permit large amounts of Cl− ions to pass, thereby hyperpolarizing the neuron more effectively and thus inhibiting the cell with greater efficiency and sensitivity. Thus this new class of channel, which is based on ChR (channel rhodopsin) but is modified to permit cations to pass through the channel rather than anions, provides yet further options. In response to blue light, this new “inhibitory” channel (iChR) will open and permit large amounts of Cl− ions to pass, thereby hyperpolarizing the neuron more effectively and thus inhibiting the cell with greater efficiency and sensitivity. When these opsins are transferred into neurons in the nervous system, those neurons can be activated or inactivated at will and with great efficiency and temporal control in response to specific wavelengths of light delivered by a light emitting device. Optogenetics therefore provides opportunities to regulate circuits with great biological specificity, so that only specific populations of neurons are activated or inhibited, without influencing nearby axons which are passing by and serve functions which are not intended targets of the therapy. This also provides opportunities for greater degree of restoration of broader circuit function by specific activating and/or inactivating multiple populations of neurons in a fashion that cannot be achieved with existing therapies.

The distribution of light in brain tissue and other targets within the central nervous system (CNS) is dictated by optical scattering by subcellular structures and absorption by endogenous chromophores, such as blood. Photomedicine often requires the illumination of a relatively large volume of tissue at or above a therapeutic threshold fluence rate. Photodynamic therapy and optogenetics are two examples of such modalities. An example of a target tissue structure is the subthalamic nucleus (STN), which occupies over 100 mm³ on average in adult humans, and about 25 mm³ in the adult rhesus macaque. However, the STN of rodents is typically <1 mm³. Thus, using a single typical end-emitting fiber optic probe may be well suited for experimental work in rodents, but not ideal for use in larger animals, such as humans, because it cannot provide both broad illumination of a clinically meaningful target and minimize adverse light induced tissue effects.

Optical diffusers would seem a good choice in lieu of a simple end emitting fiber. However, current diffusers are relatively large and inefficient for practical use in therapeutic intervention.

Existing connectors that are configured for use with multiple optical fibers are too large, bulky and unwieldy to be used routinely in clinical application.

Means to design, fabricate, deploy, and safely operate such probes are disclosed herein.

### SUMMARY

One embodiment is directed to a probe for illuminating a target tissue of a patient, comprising: a plurality of optical fibers; a probe body portion having proximal and distal ends, the probe body portion being moveably coupled to the plurality of fibers and configured to at least partially encapsulate the plurality of fibers; a distal end portion coupled to the distal end of the probe body portion, the distal end portion comprising at least one guiding feature configured to redirect a path of at least one of the optical fibers comprising the plurality of optical fibers as such at least portion of one of the optical fibers is extended through and past the distal end portion by moving the plurality of fibers relative to the probe body portion. The probe further may comprise an ejector portion configured to move the plurality of fibers relative to the probe body portion. The ejector portion may comprise an elongate member configured to advance the plurality of fibers relative to the probe body portion, the elongate portion coupled to the plurality of fibers. The elongate member may comprise an elongate structure selected from the group consisting of: a wire, a fiber, a rod, and a tube. The elongate member may comprise a polymer or metal. The probe further may comprise a collar member coupled to both the plurality of fibers and the elongate member. The ejector portion may comprise a collectively grouped portion of the plurality of fibers, and wherein the at least a portion one of the optical fibers is extended through and past the distal end portion by moving the collectively grouped portion relative to the probe body portion. At least one of the plurality of optical fibers may comprise glass or polymer. The probe body portion may comprise an at least partially circumferentially coupled member relative to the plurality of optical fibers. The probe body portion may comprise a structure selected from the group consisting of: a tube, coil, or spring. The probe body portion may comprise a tube having one or more relief cuts formed in it to increase overall structural flexibility of the tube. The one or more relief cuts may be formed in an interrupted helical pattern. The probe body portion may comprise a polymer or metal material. The probe body portion may comprise a material selected to have a relatively low friction coefficient relative to the plurality of optical fibers. The probe body portion may comprise a hydrophilic coating configured to provide relatively low friction resistance to the plurality of optical fibers when in a fluid-exposed environment. At least one of the plurality of optical fibers may comprise a pre-set shape, such that when extended through and past the distal end portion, the at least one of the plurality of optical fibers is biased to occupy such pre-set shape. The plurality of optical fibers may comprise fibers of varying lengths, such that upon extension through and past the distal end portion, they form a non-symmetric pattern. The plurality of optical fibers may be configured to be inserted into brain or spinal cord tissue structures. The probe further may comprise an infusion cannula bundled with the plurality of optical fibers, the infusion cannula having proximal and distal ends and defining a lumen therebetween. The lumen may be configured to facilitate infusion of liquid compounds from the proximal end, wherein a medical provider may have direct access, to the distal end adjacent the target tissue of the patient. The lumen may be configured to facilitate delivery of liquid compounds comprising genetic material. The plurality of optical fibers may be configured to transmit wavelengths in the range of about 400 nm to about 700 nm. The liquid compounds may comprise optogenetic material.

Another embodiment is directed to an optical diffuser, comprising a composite comprising a generally cylindrical outer shape and configured to emit light along its length through its outer surface; wherein the composite comprises a matrix material and a plurality of scattering particles embedded in the matrix material, the plurality of scattering particles having a refractive index that is different from the refractive index of the matrix material. The optical diffuser further may comprise an interface configured to provide for direct coupling between the diffuser and an optical fiber. The scattering particles may comprise microspheres. The scattering particles may comprise a material selected from the group consisting of: polytetrafluoroethylene (PTFE), polycarbonate (PC), polystyrene (PS), silicon dioxide (SiO₂), borosilicate glass, dense flint glass, soda lime glass, barium sulfate (BaSO4), titanium dioxide (TiO2), and aluminum oxide (Al2O3). The scattering particles may comprise borosilicate glass sold under the tradename BK7. The scattering particles may comprise dense flint glass sold under the tradename SF10. The matrix material may selected from the group consisting of: a polymer, a gel, an epoxy, a heat-cured material, and a light-cured material. The scattering particles may occupy a volume fraction of between about 0.1% and about 10% within the composite. The scattering particles may have a characteristic size of between about 0.10 microns and about 10 microns. The scattering particles may have a refractive index that is greater than the refractive index of the matrix material. The scattering particles may have a refractive index that is less than the refractive index of the matrix material. The diffuser further may comprise a sheath configured to at least partially encapsulate the composite. The sheath may be coupled to the composite using an adhesive. The adhesive may have a refractive index that is less than the refractive index of the matrix material. The sheath may comprise a material selected from the group consisting of: polyethylene (PE), polyethylene terephthalate (PET), polyethylene terephthalate glycol (PETG), and polytetrafluoroethylene (PTFE).

Another embodiment is directed to an optical diffuser, comprising an optical waveguide featuring a plurality of cuts configured to emit light along the length of the waveguide through an outer surface of the waveguide. The plurality of cuts may be oriented at an angle nominally perpendicular to the surface of the waveguide. The plurality of cuts may be oriented at an angle nominally oblique to the surface of the waveguide. An orientation angle of one or more of the plurality of cuts may be specifically configured to cause asymmetric diffusion of light out of the diffuser from the waveguide. The orientation angle of the plurality of cuts may be varied in a pattern to cause asymmetric diffusion of light out of the diffuser from the waveguide. The orientation angle of the plurality of cuts may be varied in a longitudinal pattern along the waveguide to cause asymmetric diffusion of light out of the diffuser from the waveguide. The orientation angle of the plurality of cuts may be varied in a pattern of discrete zones to create discrete diffuser segments. A depth of one or more of the plurality of cuts may be specifically configured to cause asymmetric diffusion of light out of the diffuser from the waveguide. The depth of the plurality of cuts may be varied in a pattern to cause asymmetric diffusion of light out of the diffuser from the waveguide. The depth of the plurality of cuts may be varied in a longitudinal pattern along the waveguide to cause asymmetric diffusion of light out of the diffuser from the waveguide. The orientation angle of the plurality of cuts may varied in a pattern of discrete zones to create discrete diffuser segments.

Another embodiment is directed to an optical connection assembly, comprising a first faceplate comprising a plurality of first fiber ports configured to provide direct contact with faces of first optical fibers coupled thereto, wherein the first fiber ports are arranged in a predetermined first two-dimensional pattern; a second faceplate comprising a plurality of second fiber ports configured to provide direct contact with faces of second optical fibers coupled thereto, wherein the second fiber ports are arranged in a predetermined second two-dimensional pattern, the second two-dimensional pattern complementary with the first two-dimensional pattern; an alignment portion configured to orient the first faceplate with the second faceplate such that the first and second two-dimensional patterns are substantially aligned; and a locking portion configured to secure a coupling between the first faceplate and second faceplate. The first two-dimensional pattern may be a regular array. The regular array may be a hexagonal array. The first fiber ports may be configured to be of different size than the second fiber ports. The first fiber ports may be configured to be smaller than the second fiber ports. The alignment portion may comprise a non-radially symmetric tongue-in-groove configuration. The locking portion may comprise at least one set of complementary interlocking teeth. The locking portion and alignment portion may be integrated into a common member. The locking portion and alignment portion may comprise a non-radially symmetric tongue-in-groove configuration with at least one set of complementary interlocking teeth.

Another embodiment is directed to an anchoring assembly for coupling a portion of a probe to the cranium of a patient, comprising a ring portion comprising one or more mounting tabs, a channel, and having an inner and outer diameter, the ring portion being configured to be permanently positioned at least partially within a hole created through the cranium of the patient, wherein the one or more mounting tabs are arranged about the outer diameter of the ring portion and configured to be positioned against an exterior surface of the cranium, and wherein the channel is configured to accommodate passage of at least a portion of the probe; a collet portion having an outer diameter and inner diameter, and defining a channel and a slot, the outer diameter being selected to engage with the inner diameter of the ring portion, wherein the channel is configured to be complementary to the channel of the ring portion and also configured to accommodate passage of at least a portion of the probe, and wherein the slot is located opposite the channel and configured to accommodate passage of at least a portion of the probe; and a cap portion comprising a cap slot that is complementary to the slot of the collet portion and sized to fit within the inner diameter of the collet portion; wherein the ring channel, collet channel, and cap slot are configured to avoid kinking the at least a portion of the probe as the it is passed, at least in part, across a wall of the cranium. The one or more mounting tabs are configured to be positioned in a location selected from those consisting of: near a bottom of the ring portion; near a top of the ring portion; and in between a bottom and a top of the ring portion. The assembly further may comprise a snap-fit feature formed within the inner diameter of the ring portion, the snap-fit feature configured to mate with the outer diameter of the collet portion. The assembly further may comprise a snap-fit feature formed within the inner diameter of the collet portion, the snap-fit feature configured to mate with the outer diameter of the cap portion. The ring portion may comprise a metal or polymer. The collet portion may comprise a metal or polymer. The cap portion may comprise a metal or polymer. The ring channel, collet channel, and cap slot may be configured to maintain a minimum bend radius of the at least a portion of the probe. The minimum bend radius of the at least a portion of the probe may be greater than or equal to 3.5 mm.

Another embodiment is directed to a therapeutic system for illuminating tissue, comprising a power supply; a controller; an illumination source operatively coupled to the controller and power supply; an applicator operatively coupled to the illumination source and also configured to engage a targeted tissue structure, the applicator configured to receive photons from the illumination source and deliver at least a portion of the received photons into the targeted tissue structure; wherein the controller is configured to control the illumination source to emit photons to the targeted tissue structure with an illumination configuration selected to avoid phototoxicity of the targeted tissue structure with prolonged use. The illumination configuration may comprise a pulsatile emission configuration configured to provide a fluence rate of less than about 55 milliwatts per square millimeter. The illumination configuration may comprise a pulsatile emission configuration configured to provide a fluence rate of greater than 55 milliwatts per square millimeter only in a volume immediately adjacent to the applicator, and less than about 55 milliwatts per square millimeter elsewhere. The pulsatile emission configuration may have a duty cycle of less than or equal to about 20%. The pulsatile emission configuration may have a duty cycle of less than or equal to about 20%. The pulsatile emission configuration may have a pulse off time of greater than or equal to about 50 milliseconds. The pulsatile emission configuration may have a pulse off time of greater than or equal to about 50 milliseconds. The pulsatile emission configuration ma have a pulse on time of less than or equal to about 20 milliseconds. The pulsatile emission configuration may have a pulse on time of less than or equal to about 20 milliseconds. The volume immediately adjacent to the applicator may comprise a thickness of less than or equal to about 300 microns. The illumination configuration may comprise a continuous emission configuration configured to provide a fluence rate of less than about 2.5 milliwatts per square millimeter. The illumination configuration may comprise a continuous emission configuration configured to provide a fluence rate of greater than 2.5 milliwatts per square millimeter only in a volume immediately adjacent to the applicator, and less than about 2.5 milliwatts per square millimeter elsewhere. The volume immediately adjacent to the applicator may comprise a thickness of less than or equal to about 300 microns. The applicator may comprise an implantable applicator. The implantable applicator may be configured to be engaged with a targeted tissue structure that comprises a portion of the nervous system. The implantable applicator may be configured to be engaged with a targeted tissue structure that comprises a nerve or a portion of the central nervous system. The targeted tissue structure may be genetically modified to encode an opsin protein. The opsin protein may be an inhibitory opsin protein. The inhibitory opsin protein may be selected from the group consisting of: NpHR, eNpHR 1.0, eNpHR 2.0, eNpHR 3.0, SwiChR, SwiChR 2.0, SwiChR 3.0, Arch, ArchT, Arch 3.0, ArchT 3.0, iChR, iC++, ChloC, Slow ChloC, iC1C2, iC1C2 2.0, and iC1C2 3.0. The opsin protein may be a stimulatory opsin protein. The stimulatory opsin protein may be selected from the group consisting of: ChR2, C1V1-T, C1V1-TT, Chronos, Chrimson, ChrimsonR, CatCh, VChR1-SFO, ChR2-SFO, ChR2-SSFO, ChEF, ChIEF, and Jaws.

### BRIEF DESCRIPTION OF THE DRAWINGS

FIGS. 1A-1D illustrate certain aspects of irradiance concepts pertaining to the subject invention.

FIGS. 2A-2C illustrate certain aspects of temporal parameters pertaining to the subject invention.

FIGS. 3 and 4 illustrate certain aspects of results of the light distribution in terms of fluence rate using a Monte-Carlo simulation of light transport.

FIG. 5 illustrates certain aspects of an effective zone of operation.

FIGS. 6A and 6B illustrate certain aspects of calculated optical distributions.

FIG. 7 illustrates an illuminated volume configuration related to the variations of FIGS. 6A and 6B.

FIGS. 8-33 illustrate certain aspects of light delivery system configurations in accordance with the present invention.

FIGS. 34A and 34B illustrate certain aspects of coupling assemblies for light delivery components in accordance with the present invention.

FIG. 35 illustrates a multiple fiber configuration in accordance with the present invention.

FIGS. 36A-36G illustrate various interface configurations that lead to losses.

FIGS. 37-41 illustrates certain aspects of light delivery system configurations in accordance with the present invention.

FIG. 42 illustrates certain aspects of a therapeutic system in accordance with the present invention.

FIGS. 43-53B illustrate certain aspects of examples and experimental data.

### DETAILED DESCRIPTION

An intracerebral probe may be comprised of multiple optical fibers that emit light from zones along their distal portions. This configuration may maximize illumination within the STN while limiting both the fluence rate and amount of tissue displaced. Multiple emitters may be used to expose clinically meaningful volumes of tissue, such as the human STN, without the risk of toxic effects, such as phototoxicity and overheating due to photothermal processes that might accompany a single emitter intended to illuminate the entire structure. Multiple emitters may also be used together in a single probe to illuminate larger volumes. A probe may be affixed to the skull using a skull anchor. These components may be used together in a photomedical system.

Major aspects of the present invention and specific teachings for better understanding them are detailed in the following sections:

1. Describing an optical distribution

2. Diffusers

3. Multi-fiber probes

4. Skull anchors

5. Multi-fiber connectors and trunk cables

6. Dosages and dosage ranges

7. Systems of the above

1. Describing an Optical Distribution:

Photonic interactions with matter, including absorption, do not necessarily depend upon the directionality of incident light. In turbid media, such as tissue, light is diffuse after only a few scattering events, while absorption is less common. Any photosensitive element within tissue is therefore illuminated from multiple angles of incidence. Thus, we are concerned with the fluence rate (a scalar) at a target location, not the irradiance (a vector) as it is commonly defined, although they turn out to have the same dimensions, as is described below.

Fluence is defined as the density of energy received upon a surface, expressed in units of [J mm⁻²]. Irradiance is defined as the density of power upon a surface, expressed in [mW mm⁻²]. These definitions typically relate to light impinging directly upon a surface, and are vector quantities. To account for the diffuse nature of light in turbid medium, like biological tissue, we define the scalar fluence as the density of energy flux through a surface, again expressed in units of [J mm⁻²]. Similarly, we define “fluence rate” as the density of energy flux rate through a surface, regardless of direction, expressed in [J mm⁻² s⁻¹], which reduces to [mW mm⁻²]. The following figure and its description detail a few critical differences between these scalar and vector quantities. The portions of the sensors that collect light are shown as unshaded.

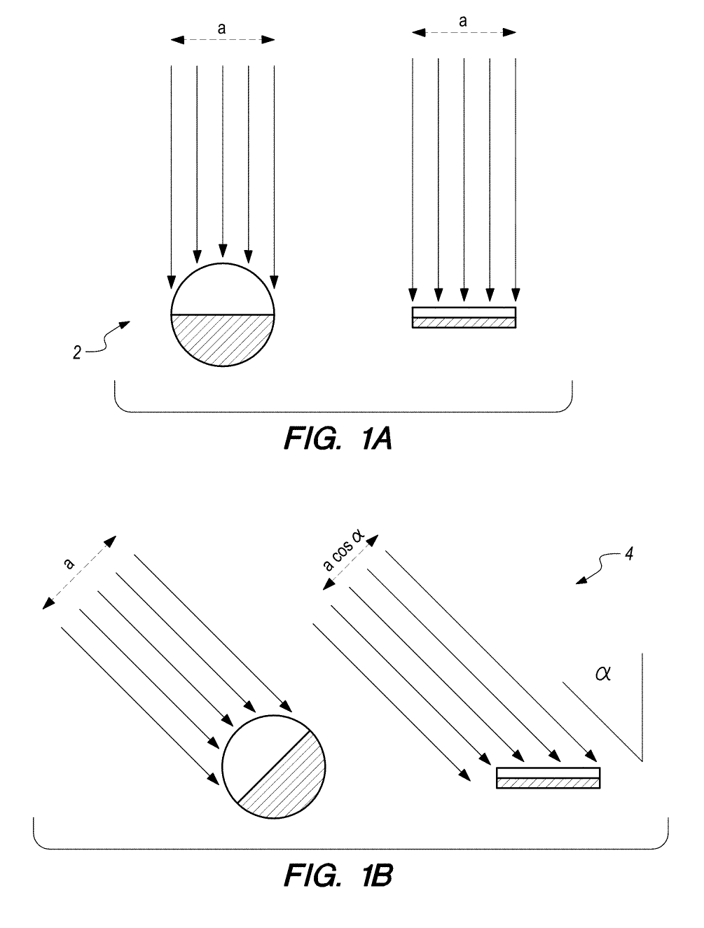

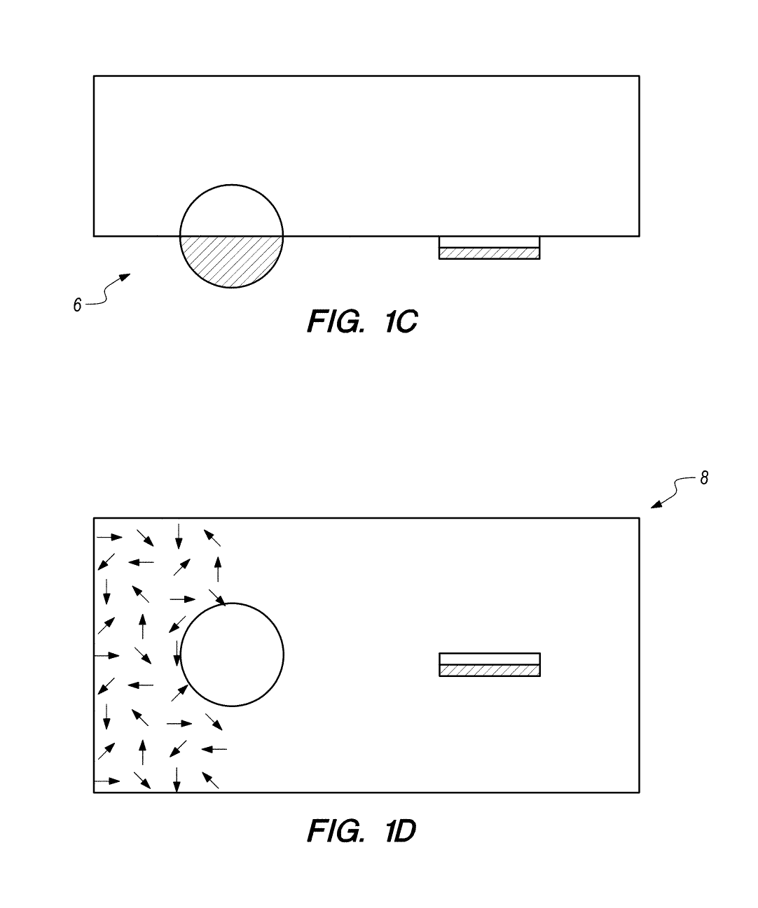

Diagram 2 of FIG. 1A shows an irradiance sensor: irradiance and fluence rate have the same numerical value. Diagram 4 of FIG. 1B shows a radiation beam at an angle to the irradiance sensor; irradiance has a smaller numerical value than fluence rate, being reduced by a factor of cos(α) due to the obliquity. Diagram 6 of FIG. 1C shows perfectly diffuse radiation from the hemisphere above the irradiance sensor; the numerical value of irradiance is exactly one half that of fluence rate because cos(α) uniformly ranges from 0 to 1 over the domain 90°≥α≥0°. Diagram 6 of FIG. 1D shows perfectly diffuse radiation from both hemispheres, both above and below the sensors: the numerical value of irradiance is one quarter that of fluence rate, wherein a spherical sensor (left) measures fluence rate, and a one-sided planar sensor (right) measures irradiance. Diagram 2 of FIG. 1A and Diagram 4 of FIG. 1B radiation is collimated and represented by parallel arrows, while Diagram 6 of FIG. 1C and Diagram 8 of FIG. 1D show radiation that is diffuse and represented by “randomly” oriented arrows.

It should be further appreciated that the concept of dosage for illumination parameters within tissue may be different than those typically reported for external photomedicine. It is also different than dosage and dosage units, as used with pharmacological intervention. Both of these cases will be handled separately.

We define the fluence rate as the spatial basis for dosage, and augment that with temporal parameters for a more accurate description of pulsatile illumination within a tissue. Temporal parameters may include, but are not limited to, a duration, a period, a frequency, an on-time, an off-time (or a “pulse interval”), and a duty cycle. Furthermore, each of these parameters may be applied to a pulse, a train of pulses (or a “burst”), a train of bursts, an overall irradiation, or a treatment. That is, temporal parameters may be selected from the list containing; a pulse duration, a pulse period, a pulse frequency, a pulse on-time, a pulse off-time, a pulse duty cycle, a burst duration, a burst period, a burst frequency, a burst on-time, a burst off-time, a burst duty cycle, a treatment duration, a treatment period, a treatment frequency, a treatment on-time, a treatment off-time, a treatment duty cycle, and combinations thereof.

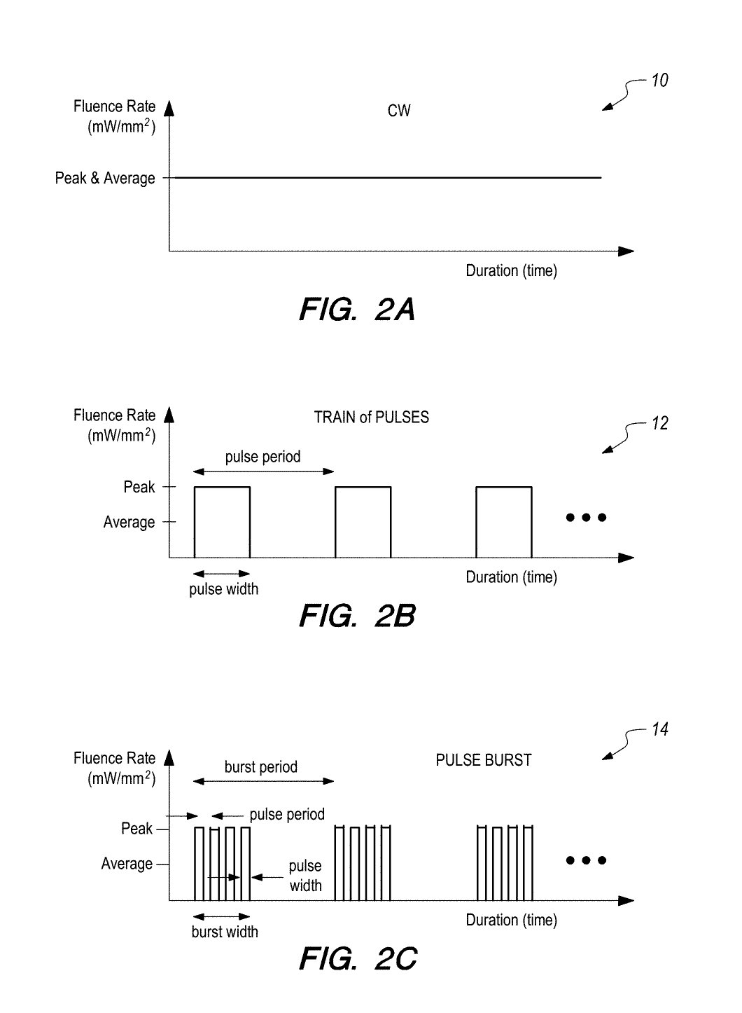

Diagram 10 of FIG. 2A shows a schematic description of temporal parameters of a continuous illumination (cw or continuous wave), and Diagram 12 of FIG. 2B shows the differences between peak power and average power for pulsatile irradiation/illumination. The fluence rate as a function of time for a CW beam is shown in Diagram 10 of FIG. 2A. Traditionally, as in the ANSI Z136 laser safety standards, the duration for a CW beam is duration >0.25 s, but from our experience with exposure to brain tissue, a more appropriate time duration to be considered continuous illumination may be duration >2 hours. The average power is then defined as the time integrated fluence rate over two hours. The peak fluence is the highest instantaneous fluence rate at any instant within the 2 hours. For a continuous beam as shown in Diagram 10 of FIG. 2A, the average and peak fluence rates are the same. An exposure using a burst is shown in Diagram 12 of FIG. 2B. In this case, the pulses are of equal amplitude and equal to the peak fluence rate. The average fluence rate is lower that the peak fluence rate due to the finite pulse interval. Another variation is a series of bursts, as shown in Diagram 14 of FIG. 2C. Each burst has a burst duration along with a burst frequency. As shown in this case the pulses are of equal amplitude and corresponds to the peak fluence rate. The average fluence rate is lower that the peak fluence rate due to the finite pulse interval and burst interval.

For clarity and to limit confusion, we will utilize the following terms to describe the dosage of pulsatile illumination herein, although other methods are considered within the scope of the present invention; a duration, an interval, and a duty cycle. We may also use the modifiers pulse, burst, and overall or treatment, if and as required. Fluence rates may be calculated using a computational optical model, as described below.

For example, a continuous wave (cw) light source (such as a laser) set to a power of 1.25 mW is configured in a system to deliver 10 ms pulses at a rate of 10 Hz, and then coupled to a standard 200 μm diameter optical fiber that delivers light to the brain. For illumination within the brain, we may say this specific configuration provides a peak fluence rate of 53 mW/mm² at a 10% duty cycle using 10 ms pulses. The fluence rate may be calculated using an optical model, as mentioned above and described in more detail below. Alternately, a continuous wave (cw) laser set to the same power of 1.25 mW that is configured to deliver 5 ms pulses in bursts of 100 pulses each with a pulse interval of 15 ms and a burst interval of 18 s would be described as having the same peak fluence rate of 53 mW/mm², but with a 5% overall duty cycle.

The following tables further illustrate the relationship between duration, interval, and duty cycle for a set of pulses, bursts of those pulses, and the overall value of duty cycle over 1 day (86400 s).

| | Duration | Interval | Frequency | Duty |
|---|---|---|---|---|
| | (ms) | (ms) | (Hz) | Cycle (%) |
| Pulse | 10 | 90 | 10 | 10 |
| Burst | 1000 | 9000 | 0.1 | 10 |
| Overall | 86400000 | — | — | 1 |

| | Duration | Interval | Frequency | Duty |
|---|---|---|---|---|
| | (ms) | (ms) | (Hz) | Cycle (%) |
| Pulse | 5 | 15 | 50 | 25 |
| Burst | 2000 | 18000 | 0.05 | 10 |
| Overall | 86400000 | — | — | 2.5 |

| | Duration | Interval | Frequency | Duty |
|---|---|---|---|---|
| | (s) | (s) | (Hz) | Cycle (%) |
| Pulse | 0.02 | 0.03 | 20 | 40 |
| Burst | 1 | 9 | 0.1 | 10 |
| Overall | 86400 | — | — | 4 |

Alternately, a “dosage unit” may also be considered in a manner similar to that used in pharmacology, where the integrated treatment energy for a given treatment duration, such as a day, for example, is described along with the temporal parameters of its delivery. An example is 10 mW of laser peak power delivered in 10 ms pulses at a 10% duty cycle may be described as a daily dosage unit of “86.4 J (Joules) delivered in 10 ms pulses at 10% duty cycle”.

For external irradiation, the size of the illuminated area, a power and a pulse duration (or an energy) are often sufficient to adequately specify spatial illumination parameters in terms of fluence or irradiance, rather than an internal fluence rate. Temporal parameters may be presented as described above.

### Tissue Optics and Computational Modeling:

The distribution of light in brain tissue is dictated by optical scattering by subcellular structures and absorption by endogenous chromophores such as blood. This is different than electrical stimulation, where bulk properties of tissue dictate the distribution of the electric field. However, predictions of therapeutic extent may be made in either case using similar computational tools, wherein an isotropic detector, such as that shown schematically in Diagram 2 of FIG. 1A, may be constructed from a single voxel of a 3- or 4-dimensional matrix that records the photon flux through its boundaries.

In such a model, tissue may be represented optically by a refractive index, n, an absorption coefficient, μₐ, a scattering coefficient, μₛ, and an anisotropy factor, g, and geometrically by a shape with bounding surfaces. The anisotropy factor may be used with the Henyey-Greenstein scatter phase function to provide a realistic distribution for expectation values of photon scattering angles, θ, in biological tissues, where ξ may be a pseudo-random number uniformly distributed between 0 and 1. These may be functions of wavelength.

$$cos ( θ ) = \frac{1}{2 g} [ 1 + {g}^{2} - \frac{1 - {g}^{2}}{1 - g + 2 g ξ} ]$$

Optical transport in turbid tissue may be expressed by the Radiative Transfer Equation (RTE), as shown below where N is the number of photons within a volume V.

$$\frac{1}{c} {\int }_{V} \frac{\partial N ( r , s , t )}{\partial t} dV = {\int }_{V} {μ}_{s} {\int }_{4 π} p ( s , {s}^{′} ) N ( r , s , t ) d {ω}^{′} dV - {\int }_{V} s \cdot ∇ N ( r , s , t ) dV - {\int }_{V} {μ}_{s} N ( r , s , t ) dV - {\int }_{V} {μ}_{a} N ( r , s , t ) dV$$

where r is the radial distance from the emitter, s the unscattered propagation direction unit vector, s′ the scattered propagation direction unit vector, c the speed of light, and p(s,s′) the Henyey-Greenstein scattering phase function.

Monte Carlo (MC) ray tracing may be used to solve the RTE. We have developed a 3D computational modeling environment utilizing FRED from Photon Engineering. The model has the flexibility to calculate the influence of complex geometries, assign parameters for each tissue type and wavelength λ and create light sources of different λ, power, and angular divergence. Rays are launched, propagated and terminated via statistical processes, and the resultant light distribution is thereby calculated.

For brain tissue, the index may not vary a great deal (e.g. 1.36≤n≤1.4) and scattering may be dominant and primarily in the forward direction (e.g. 0.8≤g≤0.9). Scattering coefficients may be large (e.g. 5 mm⁻¹≤μₛ≤50 mm⁻¹) compared to absorption (e.g. 0.01 mm⁻¹≤μₐ≤0.1 mm⁻¹). Thus, photons may undergo many scattering events before being absorbed.

In MC, computer generated random numbers allow estimation of physical quantities by sampling probability distribution functions, p[x], such as:

$$\begin{matrix}p [ x ] = {μ}_{t} {e}^{- {μ}_{t} s} \\ where \\ \frac{1}{{μ}_{t}} = \frac{1}{{μ}_{a} + {μ}_{s}}\end{matrix}$$

and the step size is

$$Δ s = - ln [ \frac{ξ}{{μ}_{t}} ]$$

Tissue optical properties are related to the probability of photon absorption and scattering per the following relations

P_{absorption}=1−e^{-μ}^{ₐ}^{Δs}, P_{scattering}=1−e^{-μ}^{ₛ}^{Δs}.

Accuracy of the model depends on the number of photons launched, N, and increases as N^{1/2}.

Using 2×10⁶ photons may achieve accurate results out to radial distances of 4 mm away from the emitter, such as may be applicable for use in modeling optical distributions within the CNS.

The presence of blood in the tissue may introduce additional absorption that is wavelength-dependent. The effect of blood on the overall absorption coefficient using the absorption coefficients for oxygenated (HbO₂) and deoxygenated (Hb) blood to modify the baseline tissue absorption coefficient using the total blood volume fraction, β, and the fractional amount of oxygenated blood, StO₂, according to the following relation may allow for improved modeling accuracy.

μₐ(λ)=β[StO₂μ_{a,HbO}_{₂}(λ)+(1−StO₂)μ_{a,Hb}(λ)]+(1−β)μ_{a,greymatter}(λ)

We chose β=4%, and StO₂=50%, which corresponds to published values for the putamen and STN. For the present disclosure, we used the following values for the RTE coefficients.

| Wavelength | μₐ | μₛ | |
|---|---|---|---|
| (nm) | (mm⁻¹) | (mm⁻¹) | g |
| 473 | 0.658 | 11.7 | 0.88 |
| 590 | 0.384 | 10.5 | 0.89 |
| 635 | 0.102 | 9.09 | 0.89 |

Single fiber end-emitters are readily available and useful for illuminating small volumes, such as may be commonly found in small animals. We have made aspects of this model available at http://optocalc.herokuapp.com/.

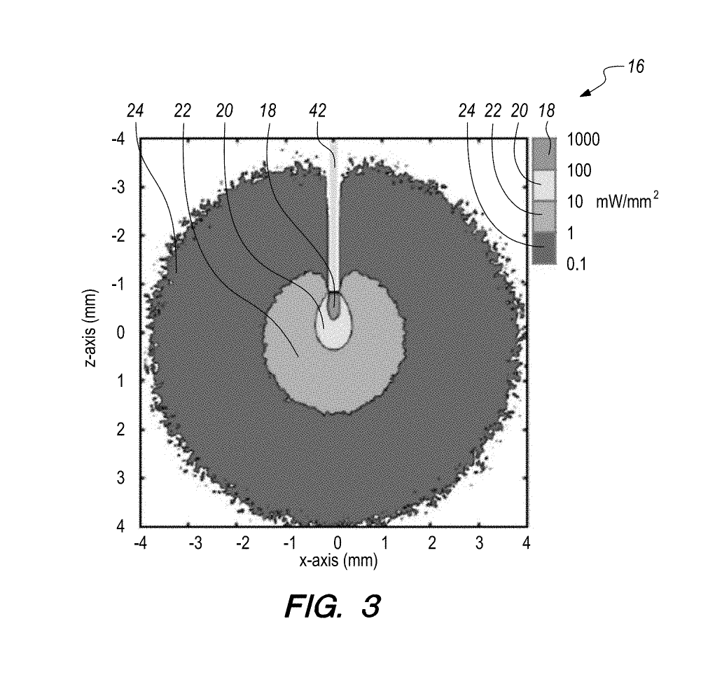

Plot 16 of FIG. 3 shows results of the light distribution in terms of fluence rate using a Monte-Carlo simulation of light transport for 10 mW of 635 nm light emitted from a single 200 μm diameter, 0.22NA fiber optic, Fiber 42, emitter embedded in grey matter containing 4% blood by volume, as is described above and expected in the human STN and other CNS tissues.

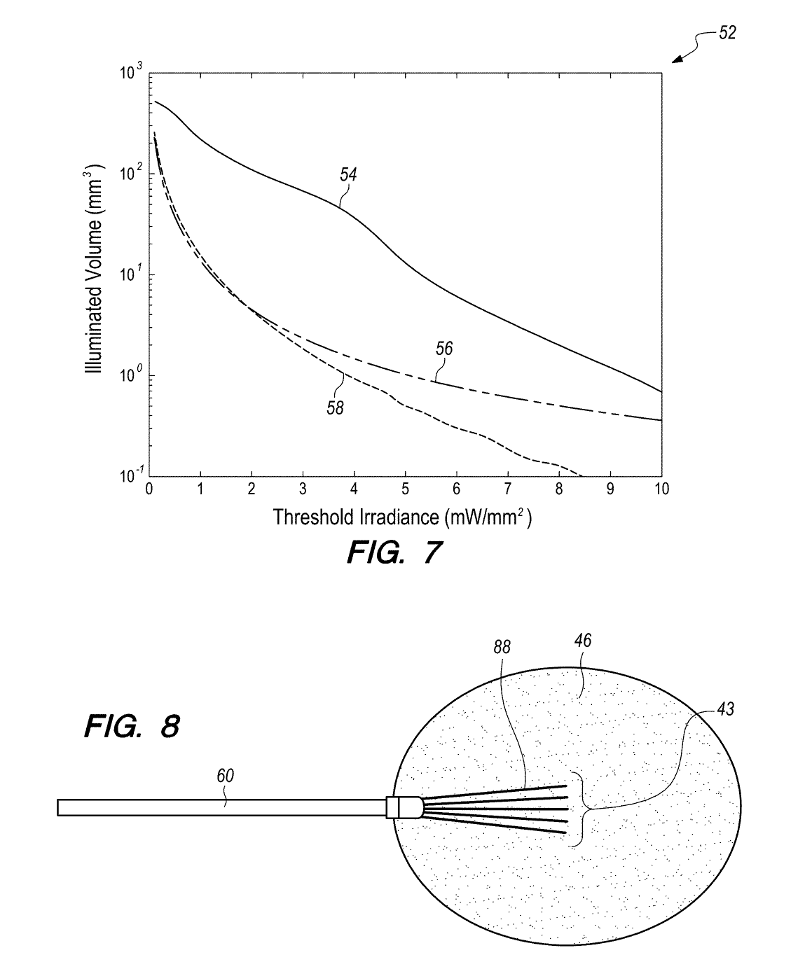

In this configuration, the power density at the fiber tip is 318 mW/mm² and builds to a peak of 415 mW/mm² at a distance of ˜400 μm distal to the fiber face due to scattering, as is indicated by region 18, which contains locations with fluence rates of between 100-1000 mW/mm². This calculation yields a 4.5 mm³ volume for fluence rates ≥2 mW/mm², and a volume=1 mm³ at fluence rates ≥5 mW/mm². These “therapeutic regions” or “therapeutic volumes” may be much smaller than is required for clinical utility. As the decade steps of the contours in the figure above show, illumination volume drops quickly as a function of opsin activation threshold fluence rate. Further defined are regions 20, 22, and 24 which are defined by the fluence rate ranges of between 10-100, 1-10, 0.1-1.0 mW/mm², respectively. The semi-logarithmic Plot 52 given in FIG. 7 illustrates this more clearly.

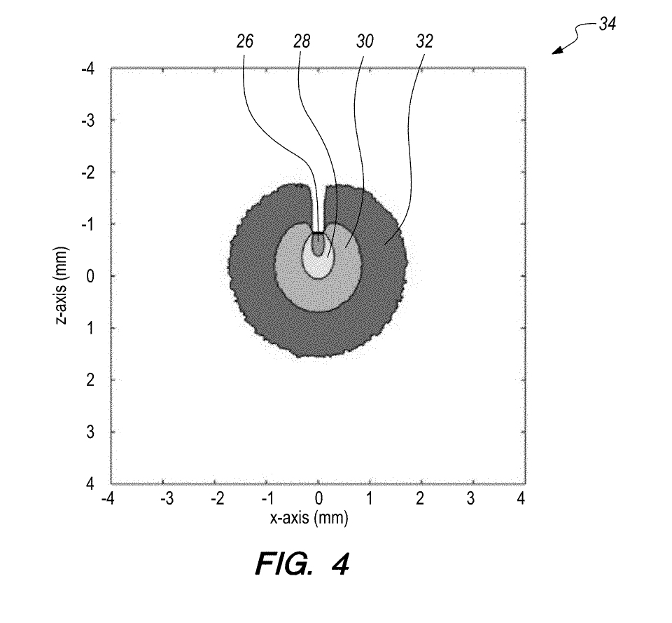

Plot 34 of FIG. 4 shows the results under the same conditions except the wavelength is changed from 635 nm to 473 nm, and designates regions 26, 28, 30, and 32 which are defined by the fluence rate ranges of between 100-1000, 10-100, 1-10, 0.1-1.0 mW/mm², respectively. In this blue light configuration, the fluence rate at the tip of Fiber 42 (not shown) is 318 mW/mm² and builds to a peak of 409 mW/mm² at a distance of ˜300 μm distal to the fiber face due to scattering. This calculation yields a 1.3 mm³ volume containing a fluence rate ≥2 mW/mm², and volume=0.4 mm³ containing a fluence rate ≥5 mW/mm². Again, these volumes may be much smaller than is required for clinical utility. Note that the volumes are smaller at the blue wavelength. This is due to the higher absorption by blood at the 473 nm relative to the 635 nm.

While this single fiber emitter approach may work well for illuminating targets within the brains of small animals like rodents, the volume of the human STN is approximately two orders of magnitude larger. To illuminate this volume at a fluence rate threshold of, say, 2 mW/mm² using 635 nm and a single fiber emitter would require 100 mW and produce a fluence rate of 4150 mW/mm², which may generate toxic effects beyond acceptable limits.

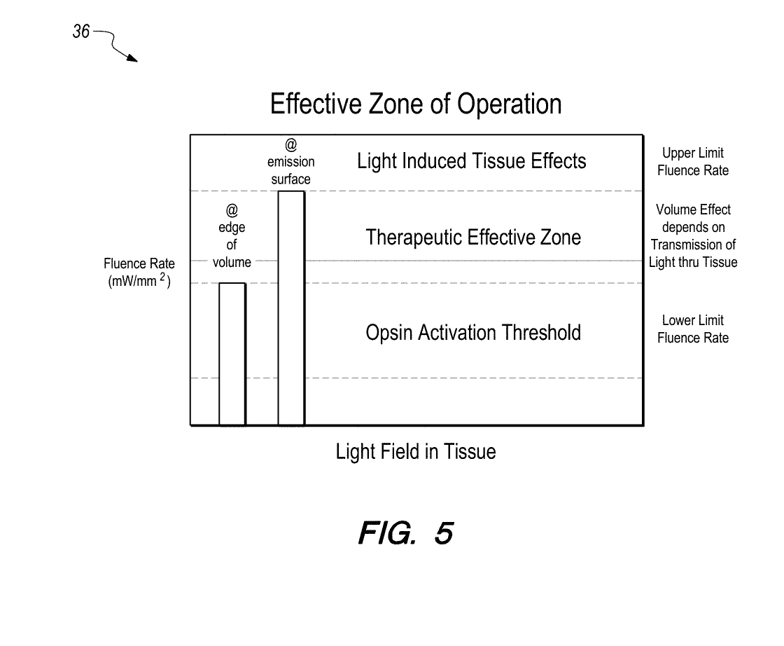

The effective zone of operation is described by diagram 36 of FIG. 5. The therapeutic effective zone is the region of tissue illuminated at fluence rate light levels above the opsin activation threshold and below the fluence levels that lead to tissue damage. This is akin to a “therapeutic window” of fluence rate. The opsin activation threshold region is represented by a band or range of numbers as opposed to a single number to represent that different opsins will have different activation thresholds. Similarly, a light induced tissue effect may be represented by a band or range of numbers rather than a single number because tissue-related effects vary with system parameters such as wavelength, duty cycle, average light power, and peak power; and also with tissue parameters such as type and the presence of blood. The therapeutic effective zone in FIG. 5 may be related to an actual volume in tissue by defining the emission geometry of the light emitters within the targeted tissue region and by the light transmission properties of the tissue itself. In general, for small separated emitters, the highest or peak fluence rate may occur at or near the emission surface of an emitter. Care must be taken to ensure that this peak value does not exceed the tissue damage limit at the emission site (note that light-induced damage may be independent from the presence of opsins). Alternately, zones of exposure beyond the damage threshold may be limited to the tissue adjacent to or nearby the emitter. The edge of the effective zone where the light level drops below the opsin activation fluence rate limit may determine the extent and therefore the volume of the tissue illuminated. The volume may also be limited by physically restricting the region modified to contain opsins by limiting the infusion volume and/or concentration.

The distances over which a fluence rate drops to the opsin activation threshold level may not be strongly affected by the size and shape of the emission. One can see that, for example, the same amount of light emitted by a single fiber may result in a higher peak fluence rate than that emitted by a uniform cylindrical diffuser, for example. In the absence of scattering, 30 mW of optical power end-emitted from a single 200 μm diameter fiber results in a peak fluence rate of 30 mW/(π×(0.1 mm)²)=955 mW/mm² at the emission face. Likewise, the same 30 mW of optical power emitted uniformly from a 200 μm diameter cylinder that is 4 mm long results in a local peak fluence rate of 30 mW/[2π×0.1 mm×4 mm]=12 mW/mm². The peak light fluence rate emitted from the cylindrical emitter or diffuser is substantially lower (˜80×) than that emitted from the end of a single fiber. The exemplary cylindrical diffuser has a larger zone of therapeutic effectiveness.

Thus, a single fiber emitter may be inadequate for illuminating large target volumes without exceeding damaging peak fluence rate levels. As used herein, the term exemplary refers to an example, rather than specifically a representation of the best configuration. Also, the terms optical distribution and light field are synonymous herein, and each may be modified to be limited in extent to comprise only that portion of an overall distribution (or field) that is above or below a certain threshold value, especially a certain value of fluence rate.

2. Diffusers:

As shown in the sections above, single fiber emitters are inadequate for illuminating large target volumes without exceeding damaging peak fluence rate levels. This can be improved by using multiple end emitting fibers but would require too many fibers to be practical. For example, using the single emission volume found above of 1.3 mm³ at 473 nm and the 200 μm diameter fiber, 77 fibers would be needed to fill a volume of 100 mm³. An alternate approach to filling large volumes at therapeutic light levels while mitigating potential toxic effects from excessive leak fluence rates a probe may be configured to use multiple fibers that may emit light along their lengths and may distribute it nominally evenly and maintain the fluence rate below about 50 mW/mm². This exemplary fluence rate limit will be described in a subsequent section.

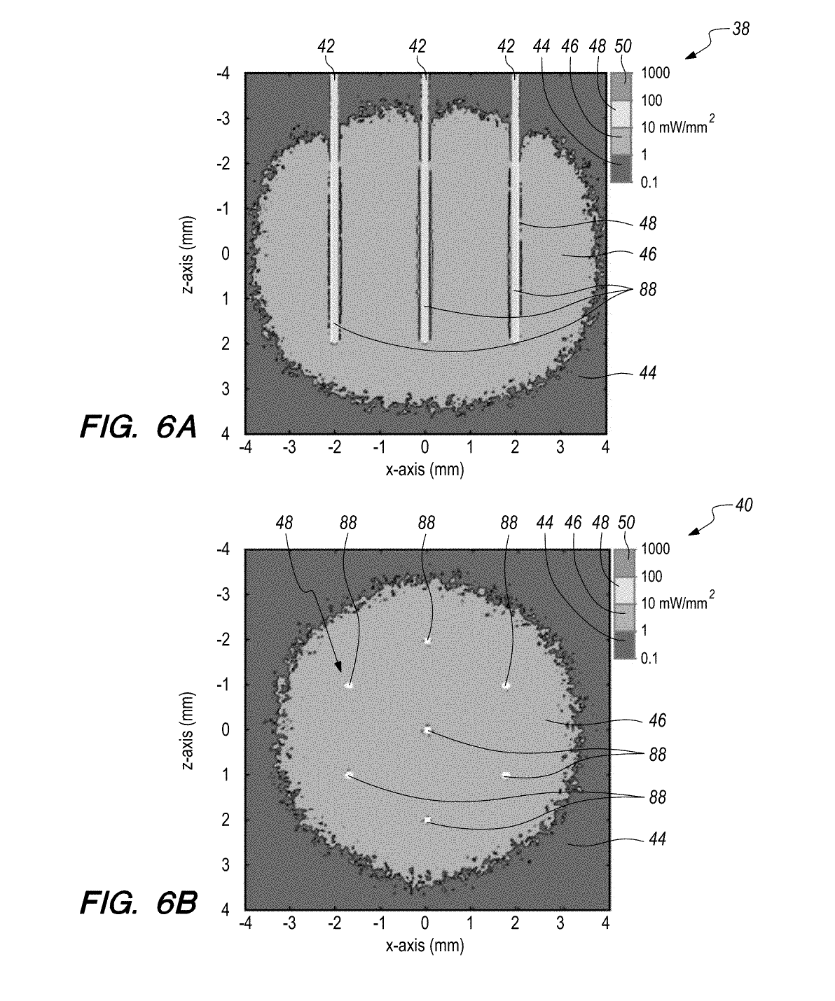

The plots 38 and 40 of FIGS. 6A and 6B, respectively, show axial and lateral views of the calculated optical distribution of an alternate embodiment comprising seven 4 mm long cylindrical diffusers 88 each emitting 30 mW of 473 nm light that can illuminate a 100 mm³ of CNS tissue as described above at fluence rates levels ≥1 mw/mm² while limiting peak fluence levels to be less than or equal to about 50 mW/mm². Diffusers 88 are located at the distal ends of optical fibers 42, and create regions 50, 48, 46, and 44; which are defined by the fluence rate ranges of between 1000-100, 100-10, 10-1, and 1-0.1, respectively. Regions 50 and 48 are very small in comparison to region 46, wherein the defined therapeutic threshold of 1 mW/mm² lies.

This configuration both distributes light more evenly than the single fiber approach and reduces the peak fluence rate. It is also less sensitive to changes in illumination volume than the single emitter shown above, as shown in plot 52 of FIG. 7.

Plot 52 of FIG. 7 illustrates the illuminated volume of the configuration described in FIGS. 6A and 6B as a function of threshold fluence rate for seven-fiber hexagonal diffuser array (line 54), showing improved performance over a single diffuser (line 58), and simple single emitter (line 56).

An exemplary embodiment of a probe consisting of multiple light diffusers 88 are located at the distal end of a light delivery system is shown in FIG. 8. In this embodiment, probe body 60 houses the seven diffusers 88 are arranged in the same pattern 43 as that of FIG. 6. Light field 46 has a peak fluence rate value near the surface of the diffusers, but slightly offset from the surface due to tissue turbidity. The edge of the effective light field is labeled as the boundary for lowest threshold. This level of the fluence rate is the minimum activation light level for the opsin. As a rule of thumb for grey matter, the peak fluence rate is approximately 1.3× the irradiance at the emission aperture and is located about 300 μm distal to it, as can be seen in the model described herein. We will use this to describe the peak fluence rates, rather than simply using the irradiance at the emission aperture or surface. Other key parameters such as the fiber diameter, the fiber separation, the emission length, and the optical power per emitter, also may need to be considered in determining the optical distribution and fluence rate levels.

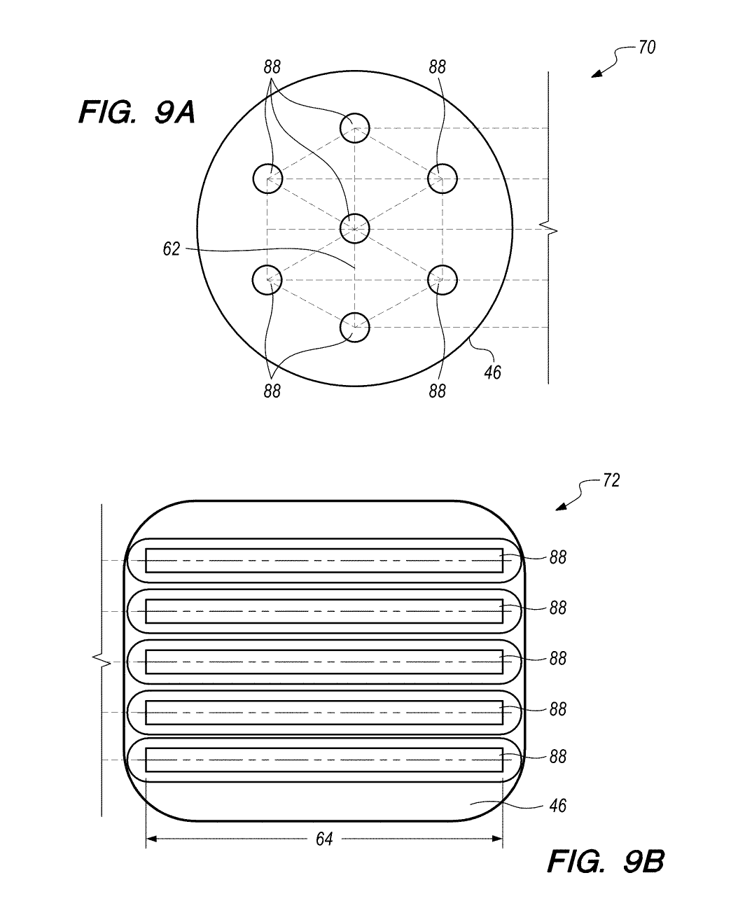

Diagrams 70 and 72 of FIGS. 9A and 9B, respectively, illustrate schematic representations of a desired illumination volume, or optical distribution, for the probe of FIG. 8. Diffusers 88 comprise an emission length 64, and a separation distance 62 to create a light field with a boundary defined by a threshold fluence rate 46. In this example, the concepts of surface fluence rate, peak fluence rate, and threshold fluence rate boundary are noted.

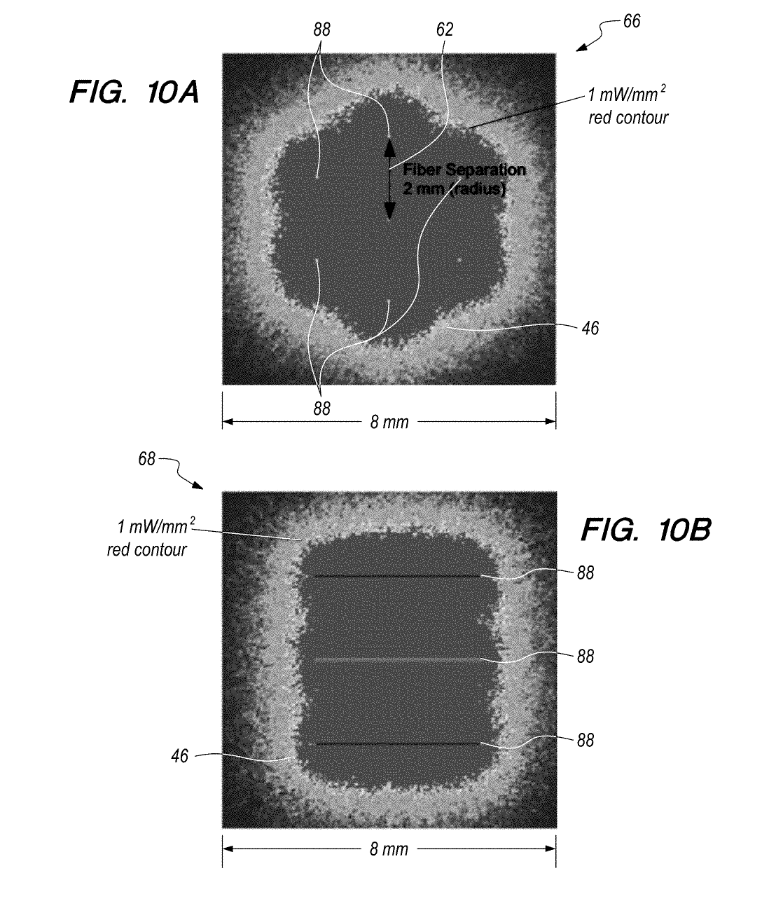

MC light transport simulation for the fluence distribution in tissue for the 7 applicator/diffuser hex pattern with a uniform emission length of 4 mm as was described with regard to in FIGS. 9A and 9B is shown in plots 66 and 69 of FIGS. 10A and 10B, respectively. Plots 66 and 68 show boundary of threshold fluence rate 46, defined in these examples as 1 mW/mm² as before, although other fluence rate definitions are possible. Likewise, separation distance 62 in this example is 2 mm, although other separation distances are possible.

The parameters used in the simulation are shown in the following tables.

SIMULATION INPUT

| | |
|---|---|
| 700,000 | rays |
| 7 | Diffusers |
| 28 | mW per diffuser |
| 196 | mW total |

GREY MATTER,
4% BLOOD

λ = 473 nm
μₛ = 11.7 mm⁻¹
g = 0.89
μₐ = 0.658 mm⁻¹

The results of the simulation are shown in the table below; wherein the volume corresponding to the 1 mW/mm2 contour is over 100 mm3, and the peak fluence rate is approximately at the 50 mW/mm2 level.

SIMULATION OUTPUT
Peak Fluence Rate: 53 mW/mm²

| Fluence Rate Cutoff | Volume |
|---|---|
| 0.5 mW/mm² | 149 mm³ |
| 1 mW/mm² | 107 mm³ |
| 2 mW/mm² | 66 mm³ |

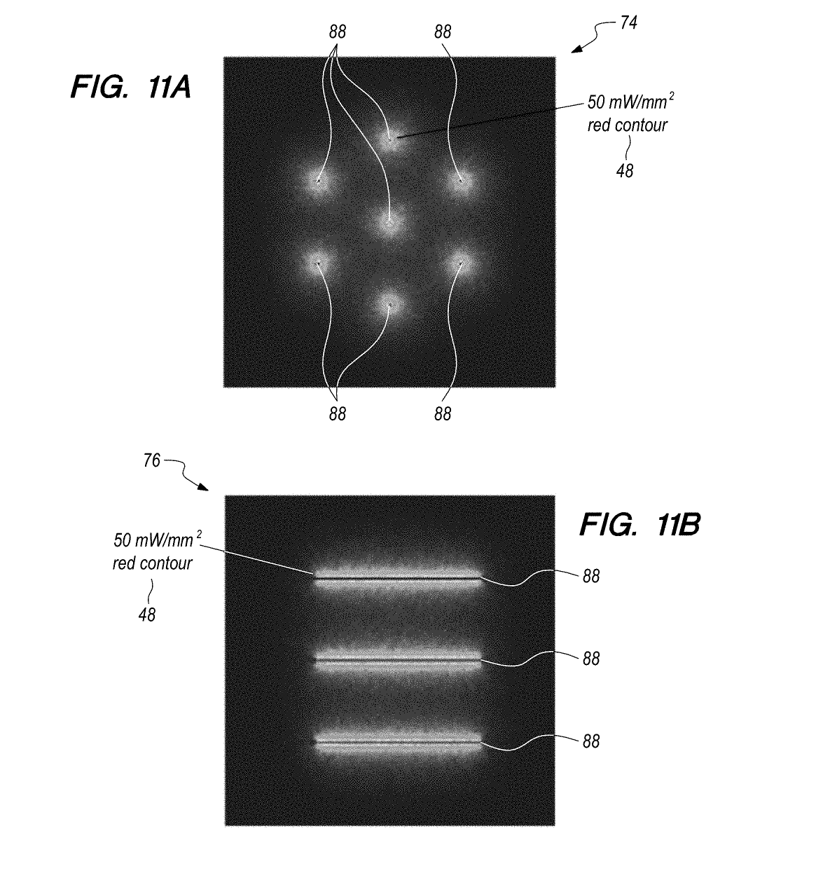

Plots 74 and 76 of FIGS. 11A and 11B, respectively, show the same light field fluence distribution as that of FIGS. 10A and 10B but with the data scaled to make the volumes containing fluence rate levels above 50 mW/mm² more visible. Region 48 describes volumes containing fluence rates ≥50 mW/mm² and can be seen to occupy only a tiny space immediately adjacent to the emission from diffusers 88. Note that in these figures, these regions are sparse and located near the diffuser emission surfaces.

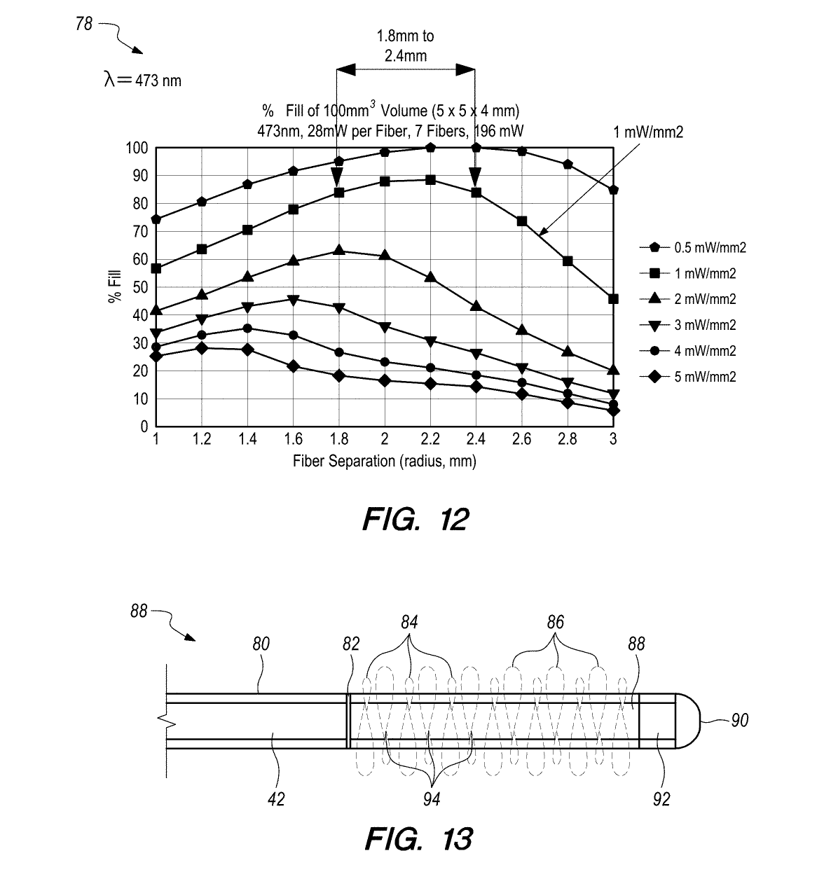

Plot 78 of FIG. 12 shows the calculated efficiency in filling a continuous 100 mm² volume as a function of the fiber separation for the configuration shown in FIGS. 11A and 11B for various threshold levels.

In this exemplary configuration, a volume fill factor of >80% for region 46 may be achieved with a fiber separation of between 1.8 to 2.4 mm. Of course, similar treatments may be applied to alternate configurations, desired fill factors, and threshold levels to generate designs thereby.

By way of nonlimiting example, for the thresholds determined for 473 nm light that are shown in the following table, dynamic ranges of 500× for cw irradiation and 26× for pulsatile irradiation are deduced by applying the ratio of Damage/Therapeutic Thresholds.

| | CW | Pulsatile |
|---|---|---|
| Fluence Rate, Damage | 2.1 | 55 |
| (mw/mm²) | | |
| Fluence Rate, Tx | 0.004 | 2.1 |
| (mw/mm²) | | |
| Dynamic range | 500 | 26 |

The values for fluence rates may be calculated by using the distance between probe and target and the powers as input to the optical models defined elsewhere herein. The dynamic range as defined may be considered a guiding value for dosage protocols and translational probe emitter design by providing power limits for the exposure level at or near the probe surface and the volume obtained thereby, as has been described elsewhere herein. Such considerations may provide for system specifications or operational boundaries. For example, using the above listed values for fluence rates and commensurate dynamic ranges yields the following system specification for the same 7× fiber diffuser described with respect to FIGS. 9-12, with the additions of the wavelength now being 473 nm instead of 635 nm and the diffuser length being 6 mm instead of 4 mm and operated at a 10% duty cycle.

| | CW | Pulsed |
|---|---|---|
| Fluence rate, peak | 2.1 | 55 |
| (mW/mm²) | | |
| Fluence rate, threshold | 0.004 | 2.1 |
| (mW/mm²) | | |
| Dynamic range | 500 | 26 |
| Illuminated volume (mm³) | 111 | 17 |
| Power/Fiber, peak (mW) | 2.9 | 75 |
| Power/Fiber, avg. (mW) | 2.9 | 7.5 |
| Emitters required to | 1 | 6 |
| fill 100 mm² | | |
| Total optical power, | 2.9 | 450 |
| peak (mW) | | |
| Total optical power, | 2.9 | 45 |
| avg. (mW) | | |

Thus, an illumination system, such as an embedded illumination system for targeting tissue in the CNS, may be defined and configured for illuminating clinically meaningful volumes of genetically modified tissue using multiple diffusers without otherwise engendering further risks to the patient beyond those encountered with common deep brain stimulation devices.

Likewise, the same analysis for a simple end-emitting 200 μm core diameter fiber optic yields the following table which shows that simple end-emitting fiber optics are insufficient to illuminate clinically meaningful volumes of tissue, e.g. a 100 mm³ STN;

| | CW | Pulsed |
|---|---|---|
| Illuminated volume (mm³) | 3.5 | 0.1 |
| Power/Fiber, peak (mW) | 0.05 | 1.3 |
| Power/Fiber, avg. (mW) | 0.05 | 0.13 |
| Emitters required to | 29 | 1000 |
| fill 100 mm² | | |
| Total optical power, | 1.45 | 1300 |
| peak (mW) | | |
| Total optical power, | 1.45 | 130 |
| avg. (mW) | | |

FIG. 13 illustrates an embodiment of a diffuser 88 that emits over its surface in a manner similar to that utilized in the previous simulations. This technique may employ cuts 94 in a plastic optical fiber 42 (POF), or monofilament to create diffuser 88 that joins the delivery fiber at fiber-diffuser interface 82. Junction 82 may be held in place using an epoxy that matches the refractive index of the fiber and/or diffuser to improve overall transmission. Examples of epoxies suitable for use with a silica fiber and a POF diffuser are Norland 61 and Norland 85, which have refractive indices at this wavelength of 1.57 and 1.46, respectively. Alternately, cuts may be made in harder material by using a dicing saw, or even pulsed laser micromachining. This cut optic may be embedded into capillary tube 80 and placed at the distal end of delivery fiber 42. A portion of the light that traverses the diffuser may be scattered by each cut, which is shown as Diffuse Light Fields DLF1 84 and Diffuse Light Fields DLF2 86. Cuts 94 may provide light fields 84 that emit from the same location on the surface as cuts 94. Cuts 94 may provide light fields 86 that emit from a location on the surface opposite from cuts 94. Diffuse Light Fields DLF2 86 may typically output couple more power than Diffuse Light Fields DLF1 84. The light emitted from each cut 94 may expand and overlap that from an adjacent cut 94 such that the light is nominally uniform when it encounters tissue. The entire assembly may be potted using a material with a sufficiently different index of refraction than that of the monofilament to maintain the optical output coupling of the cuts. The potting/embedding material may enter the cuts in the monofilament. Using an embedding material with the same index of refraction may simply render the cuts largely moot, reducing their effectiveness at coupling light from the probe and into tissue. Likewise, adjusting the refractive index of the potting material may allow for adjustment of the output coupling from the cuts to affect the overall distribution of the diffuser. A distal space 92 may be provided in between the end face of diffuser 88 and end cap 90. Distal space 92 may allow any light still guided along diffuser 88 to expand prior to encountering tissue and thus reduce its exposure level.

Alternately, the pitch of cuts 94 along the length of diffuser 88 may be chosen such that the output from each cut overlaps that of its neighbors. The depth and/or angle of cuts 94 may also be altered along the length and/or circumference of the diffuser. A further alternate embodiment utilizes modulation of the pitch and depth to provide a nominally uniform output coupling density per unit length of diffuser.

For example, a tri-polymer monofilament of 150 μm OD may be processed to include a series of cuts through its outer surface that move progressively deeper along the length of the waveguide.

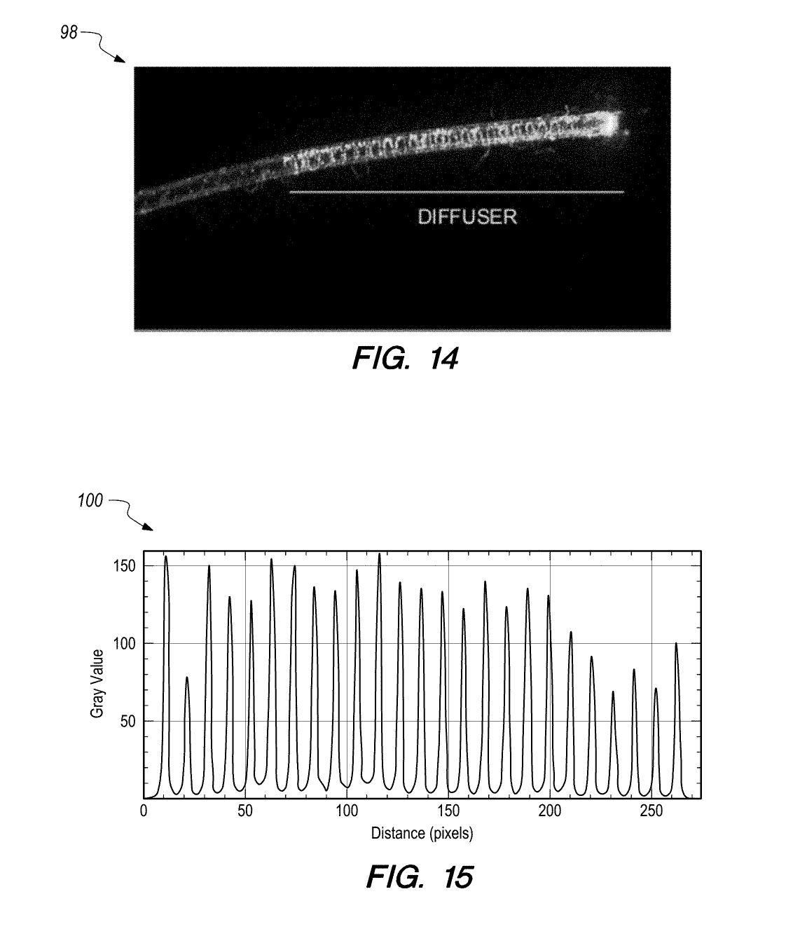

Image 98 of FIG. 14 shows an example of such a diffuser utilizing nominally 32 individual cuts 94 that were made at a constant pitch of 125 μm and oriented to be perpendicular to both the outer surface and long axis of a 125 μm core diameter plastic optical fiber. The cut depth increases from an initial depth of 15 μm at its proximal end to a final cut depth of 25 μm at its distal end. This cut pattern was produced along a first region and the fiber then rotated about its long axis by 90° and reproduced with a 20 μm lateral offset to avoid overlapping cuts from the first region. This process was then iterated twice more to ultimately produce a diffuser configured with 4 cut regions to create a nominally 4 mm diffuser length. The varying cut depth may be intended to capture light from different radial distances within the fiber/waveguide, and be dependent upon the mode properties of the waveguide/For example, shallow cuts may tend to output couple light from higher order modes within a waveguide, while deeper cuts may tend to output couple light from lower order modes. A positive cut depth gradient (cuts that increase in depth along the direction of average optical propagation) may thus output couple light first from higher order mode, and a negative cut depth gradient may thus output couple light first from both lower and higher order modes.

Plot 100 of FIG. 15 shows the intensity output profile along the length of the diffuser of FIG. 14. In this specific case, a telecentric lens was used with a CMOS camera at a resolution of 16 μm/pixel to image the outer surface of the diffuser in air to determine the uniformity of its output per cut, as seen by the peaks in the plot. Although the peaks are discrete and show close to 100% modulation depth, this configuration may be nonetheless sufficient uniform to be suitable for use in tissue, as the intrinsic turbidity of tissue ameliorates some of the discrepancies in uniformity. Relatively small discrepancies may be on the order of 50% for CNS targets. It should be noted that the optical distribution in air may not be representative of that in tissue and that the modeling described herein may provide the expected distribution and tolerances for qualifying samples in air rather than tissue.

A further alternate embodiment utilizes a configuration wherein Distal Space 92 between diffuser 88 and End Cap 90 of FIG. 13 is at least partially filled with either a scattering material and/or a retroreflective material that may serve to couple any remaining light from the diffuser core into tissue directly, or alternately back into the diffuser itself. Examples of such scattering materials are described in the following section. A suitable retroreflective material is, by way of nonlimiting example, BaTiO₃, such as part number P2453BTA-4.3 from Cospheric, Inc.

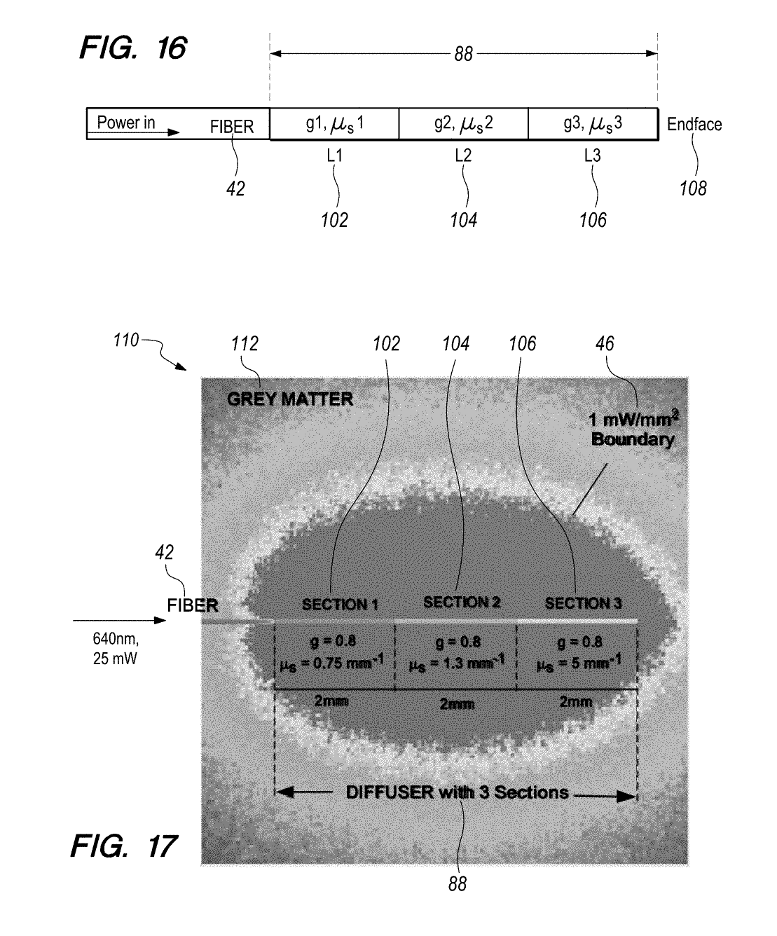

An alternate method for fabricating a diffuser is shown in FIG. 16. In this embodiment, 3 separate sections 104, 106, and 108 are attached to the end delivery fiber 42 to form diffuser 88. This attachment can be achieved using a sheath such as a capillary or other tubing to contain the segments (not shown). The sheath may be configured to engage the outer surface of both the diffusers and the optical fiber supplying light to the diffuser. It may further be configured to provide for an adhesive between the outer surfaces of the fiber and/or diffuser segments. Each section of the diffuser may have different scattering properties that when configured achieve a nominally uniform emission along the length and ideally emission out of the distal endface 108 with an irradiance less than or similar to that of the cylindrical surface of diffuser 88. Sections 102, 104, and 106 may be characterized by their scattering parameters as defined earlier herein; g₁, μₛ₁; g₂, μₛ₂; g₃, μₛ₃, respectively. The following table details the scattering parameters for this segmented diffuser when operated at 635 nm in grey matter.

| Segment | μ_{s′} (mm⁻¹) |
|---|---|
| 102 | 0.15 |
| 104 | 0.26 |
| 106 | 1.0 |

The different scattering properties can be achieved by embedding microparticles such as glass microspheres or TiO₂ particles into an embedding or encapsulating medium, such as, but not limited to, a heat- or photo-curable epoxy. The scattering properties may be adjusted or tailored by the choice of particle size, particle refractive index, particle volume concentration, and the optical performance predicted using a Mie Scattering model, as described herein.

An example of the scattering values from the example of FIG. 16 is shown in plot 110 of FIG. 17, wherein delivery fiber 42 conveys light to diffuser 88, which is comprised of segments 102, 104, and 106. Diffuser 88 and a distal portion of fiber 42 are implanted within grey matter 112 and produce a light field described by region 46, defined as before by the boundary of 1 mw/mm². Although not shown, a configuration like that above which comprises an enclosing capillary tube may also be used.

This exemplary simulation was performed at a wavelength of 640 nm but similar results may be achieved at other wavelengths. In the figure, the 1 mW/mm² fluence rate area is represented in red. Combinations of different particles and encapsulant/embedding media are possible, and a Mie Scattering model may be used to predict optical properties.

The key parameters for Mie calculations are the coefficients aₙ and bₙ to compute the amplitudes of the scattered field, and cₙ and dₙ for the internal field, respectively. Herein we utilize the mathematical notational conventions of Bohren C. F. and D. R. Huffman, Absorption and Scattering of Light by Small Particles (John Wiley, New York, N.Y., 1983), which is incorporated by reference herein in its entirety.

$${a}_{n} = \frac{{m}^{2} {{j}_{n} ( mx ) [ {xj}_{n} ( x ) ]}^{′} - {μ}_{i} {{j}_{n} ( x ) [ {mxj}_{n} ( mx ) ]}^{′}}{{m}^{2} {{j}_{n} ( mx ) [ {xh}_{n}^{( 1 )} ( x ) ]}^{′} - {μ}_{1} {{h}_{n}^{( 1 )} ( x ) [ {mxj}_{n} ( mx ) ]}^{′}}$$

$${b}_{n} = \frac{{μ}_{i} {{j}_{n} ( mx ) [ {xj}_{n} ( x ) ]}^{′} - {{j}_{n} ( x ) [ {mxj}_{n} ( mx ) ]}^{′}}{{μ}_{1} {{j}_{n} ( mx ) [ {xh}_{n}^{( 1 )} ( x ) ]}^{′} - {{h}_{n}^{( 1 )} ( x ) [ {mxj}_{n} ( mx ) ]}^{′}}$$

$${c}_{n} = \frac{{μ}_{1} {{j}_{n} ( x ) [ {xh}_{n}^{( 1 )} ( x ) ]}^{′} - {μ}_{1} {{h}_{n}^{( 1 )} ( x ) [ {xj}_{n} ( x ) ]}^{′}}{{μ}_{1} {{j}_{n} ( mx ) [ {xh}_{n}^{( 1 )} ( x ) ]}^{′} - {{h}_{n}^{( 1 )} ( x ) [ {mxj}_{n} ( mx ) ]}^{′}}$$

$${d}_{n} = \frac{{μ}_{1} {{mj}_{n} ( x ) [ {xh}_{n}^{( 1 )} ( x ) ]}^{′} - {μ}_{1} {{mh}_{n}^{( 1 )} ( x ) [ {xj}_{n} ( x ) ]}^{′}}{{m}^{2} {{j}_{n} ( mx ) [ {xh}_{n}^{( 1 )} ( x ) ]}^{′} - {μ}_{1} {{h}_{n}^{( 1 )} ( x ) [ {mxj}_{n} ( mx ) ]}^{′}}$$

where m is the refractive index of the sphere relative to the ambient medium, x=ka the size parameter, a the radius of the sphere and k=2π/λ is the wave number, λ the wavelength in the ambient medium, and μ₁ the ratio of the magnetic permeability of the sphere to the magnetic permeability of the ambient medium. The functions jₙ(z) and h⁽¹⁾ₙ(z)=jₙ(z)+iyₙ(z) are spherical Bessel functions of order n=1, 2, . . . and of the given arguments, z=x or mx, respectively, and primes mean derivatives with respect to the argument. The derivatives follow from the spherical Bessel functions themselves, namely

[zjₙ(z)]^{t}=zjₙ₋₁(z)−njₐ(z);[zhₙ⁽¹⁾(z)]′=zhₙ₋₁⁽¹⁾(z)−nh_{R}⁽¹⁾(z)

Often μ₁=1, and the following simplification is justified.

$${a}_{n} = \frac{{m}^{2} {{j}_{n} ( mx ) [ {xj}_{n} ( x ) ]}^{′} - {{j}_{n} ( x ) [ {mxj}_{n} ( mx ) ]}^{′}}{{m}^{2} {{j}_{n} ( mx ) [ {xh}_{n}^{( 1 )} ( x ) ]}^{′} - {{h}_{n}^{( 1 )} ( x ) [ {mxj}_{n} ( mx ) ]}^{′}} ; \\ {b}_{n} = \frac{{{j}_{n} ( mx ) [ {xj}_{n} ( x ) ]}^{′} - {{j}_{n} ( x ) [ {mxj}_{n} ( mx ) ]}^{′}}{{{j}_{n} ( mx ) [ {xh}_{n}^{( 1 )} ( x ) ]}^{′} - {{h}_{n}^{( 1 )} ( x ) [ {mxj}_{n} ( mx ) ]}^{′}}$$

$${c}_{n} = \frac{{{j}_{n} ( x ) [ {xh}_{n}^{( 1 )} ( x ) ]}^{′} - {{h}_{n}^{( 1 )} ( x ) [ {xj}_{n} ( x ) ]}^{′}}{{{j}_{n} ( mx ) [ {xh}_{n}^{( 1 )} ( x ) ]}^{′} - {{h}_{n}^{( 1 )} ( x ) [ {mxj}_{n} ( mx ) ]}^{′}} ;$$

$${d}_{n} = \frac{{{mj}_{n} ( x ) [ {xh}_{n}^{( 1 )} ( x ) ]}^{′} - {{mh}_{n}^{( 1 )} ( x ) [ {xj}_{n} ( x ) ]}^{′}}{{m}^{2} {{j}_{n} ( mx ) [ {xh}_{n}^{( 1 )} ( x ) ]}^{′} - {{h}_{n}^{( 1 )} ( x ) [ {mxj}_{n} ( mx ) ]}^{′}}$$

MATLAB (MathWorks, Natick, Mass.) may be used to construct a Mie Scattering model using this mathematical framework, and made to output the optical parameters μₛ, g, and μ_{s′}

$$\begin{matrix}{σ}_{s} = {AQ}_{sca} \\ ρ = \frac{{v}_{f}}{{v}_{particle}} \\ {μ}_{s} = {ρσ}_{s}\end{matrix}$$

wherein A is the geometrical cross section of the scattering particle, fᵥ is the volume fraction of particles in the mixture, V_{particle} is the particle volume, and Q_{sca} is given by

$${Q}_{sca} = \frac{2}{{x}^{2}} {\sum }_{n = 1}^{∞} ( 2 n + 1 ) ( {\vert {a}_{n} \vert}^{2} + {\vert {b}_{n} \vert}^{2} )$$

The scattering anisotropy, g= [symbol] cos(theta) [symbol] is accounted for using the following relations

$$\begin{matrix}{μ}_{{s}^{′}} = {μ}_{s} ( 1 - g ) = {ρσ}_{s} ( 1 - g ) \\ {Q}_{sca} 〈 cos θ 〉 = \frac{4}{{x}^{2}} \{ {\sum }_{n = 1}^{∞} \frac{n ( n + 2 )}{n + 1} Re ( {a}_{n} {a}_{n + 1}^{′} + {b}_{n} {b}_{n + 1}^{′} ) + {{\sum }_{n = 1}}^{∞} \frac{2 n + 1}{n ( n + 1 )} Re ( {a}_{n} {b}_{n}^{*} ) \} \end{matrix}$$

Materials for the formulations to achieve the specified scattering properties are shown in the following tables.

### Exemplary Epoxy Encapsulants/Embedding Materials.

| | Part | | Density | Viscosity |
|---|---|---|---|---|
| Mfg | Number | nd | (g/ml) | (cP) |
| Norland | 1315 | 1.32 | 1.01 | 15 |
| Dymax | 1404-M-UR | 1.47 | 1.07 | 6,000 |
| Dymax | 1206-M-SC | 1.47 | 1.05 | 300 |
| Dymax | 208-CTH-F | 1.49 | 1.01 | 225 |
| Dymax | 1165-M | 1.50 | 1.05 | 11,500 |
| Dymax | 431 | 1.50 | 1.04 | 500 |
| Dymax | 1161-M | 1.51 | 1.04 | 300 |
| Dymax | 204-CTH-F | 1.51 | 1.00 | 6,500 |
| Norland | 61 | 1.56 | 1.23 | 400 |
| Norland | 63 | 1.56 | 1.72 | 2,000 |
| Norland | 85 | 1.46 | 1.23 | 200 |
| Norland | 164 | 1.64 | 1.55 | 80 |
| Epotek | 301 | 1.52 | 1.09 | 150 |

### Exemplary Particles/Microspheres.

| | | | ø min | |
|---|---|---|---|---|
| Mfg | Part Number | Mat'l | (μm) | nd |
| Cospheric | MTL/ISO-T | Hollow | 5.00 | 1.100 |
| | | Glass | | |
| Polysciences | Microdispers- | PTFE | 0.20 | 1.310 |
| | 200 | | | |
| Polysciences | Microdispers- | PTFE | 3.00 | 1.310 |
| | 3000 | | | |
| Cospheric | SiO2MS-1.8 | SiO2 | 0.05 | 1.457 |
| | 0.166 um | | | |
| Cospheric | PMPMS-1.4 3-10 um | PMMA | 3.00 | 1.489 |
| Cospheric | PENS-XXX | PE | 0.25 | 1.500 |
| CENOSTAR | CenoBar 10 or | BaSO4 | 2.00 | 1.636 |
| | E10 | | | |
| Corpuscular | 130150-10 | Al2O3 | 0.10 | 1.766 |
| Cospheric | BTGMS-XXX | BaTiO3 | 5.00 | 2.426 |
| Corpuscular | 220150-10 | TiO2 | 0.10 | 2.582 |

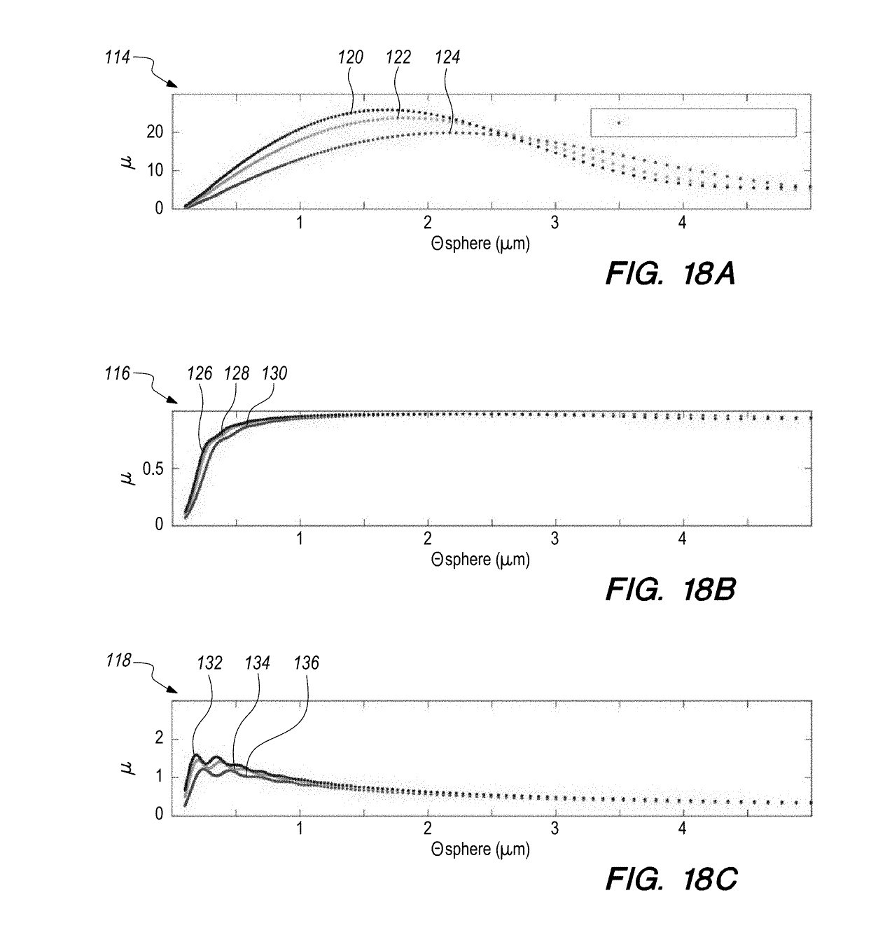

For example, plot 114 of FIG. 18A illustrates the expected results for μₛ of a 1% fᵥ of SiO₂ microspheres embedded in Norland N1315 epoxy as a function of sphere diameter for wavelengths of 488 (line 120), 532 (line 122), and 635 nm (line 124) per the above described Mie model. Similarly, plot 116 of FIG. 18B illustrates the expected results for g of a 1% fᵥ of SiO₂ microspheres embedded in Norland N1315 epoxy as a function of sphere diameter for wavelengths of 488 (line 126), 532 (line 128), and 635 nm (line 130) and plot 118 of FIG. 18C illustrates the expected results for μ_{s′} for wavelengths of 488 (line 132), 532 (line 134), and 635 nm (line 136).

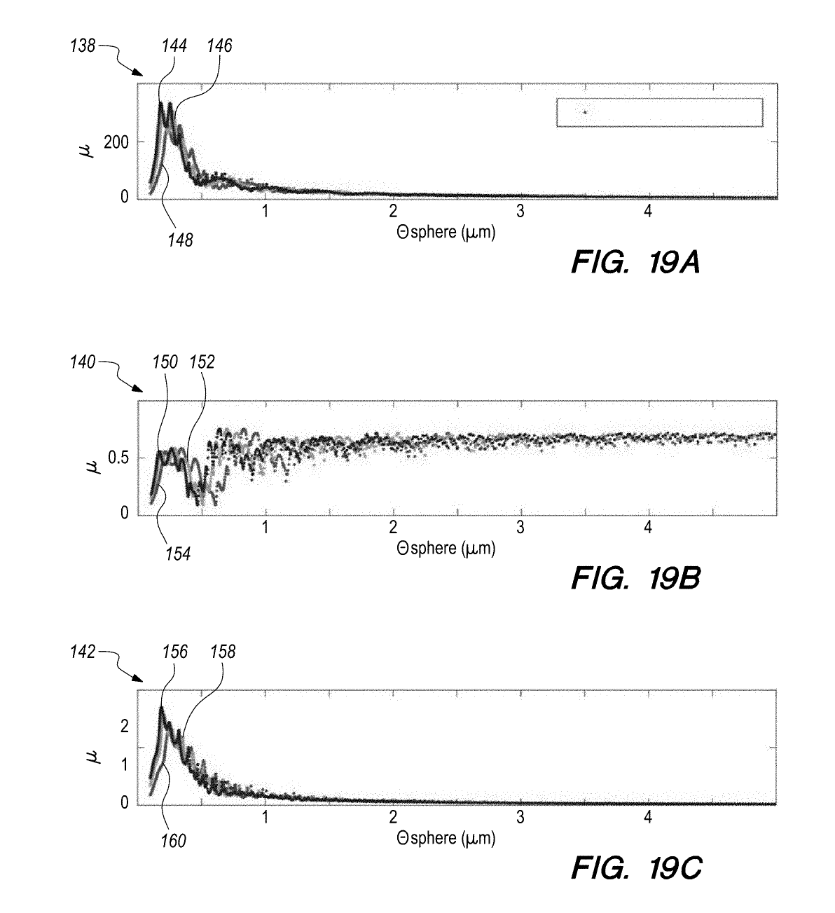

Plot 138 of FIG. 19A illustrates the expected results for μₛ of a 1% fᵥ of TiO₂ particles embedded in Norland N1315 epoxy as a function of sphere diameter for wavelengths of 488 (line 144), 532 (line 146), and 635 nm (line 148) per the above described Mie model. Similarly, plot 140 of FIG. 19B illustrates the expected results for g of a 1% fᵥ of SiO₂ microspheres embedded in Norland N1315 epoxy as a function of sphere diameter for wavelengths of 488 (line 150), 532 (line 152), and 635 nm (line 154) and plot 142 of FIG. 19C illustrates the expected results for μ_{s′} for wavelengths of 488 (line 156), 532 (line 158), and 635 nm (line 160).

Scatterers may be dispersed within the uncured epoxy directly, or through the use of a thinning agent, such as xylene or acetone to provide for easier dispersion of the particles in an otherwise viscous medium. Scattering particles may further be treated with surfactants to provide more homogenous mixing and particle distribution. Exemplary surfactants include, but are not limited to; Tween 20, Triton X-100, SDS, Poloxamer 181, CTAB, AOT, and Calgon. These may be introduced by immersing the particles in an aqueous solution of between 0.1%-1% surfactant that is gently mixed at room temperature for between 2-12 hours and then the water removed by methods such as centrifugation, compression pelletization, or drying; by way of nonlimiting examples. While surfactants may serve to more uniformly distribute the scattering particles in the embedding medium, it should be noted that their use is not strictly required. Even mixtures with agglomerated, flocculated, and/or aggregated particles may still function as a diffuser. In such cases, the effective diameter may be ascertained through microscopy for predictive modeling or samples measured to empirically determine μ_{s′} and fᵥ may then be iterated as appropriate. Mixing may be achieved using a high shear mixer and/or sonication to distribute the scatterers, which may also be performed at least partially during evaporation. Vacuum evaporation may be used to remove at least some of the thinning agent, the relationship between boiling point and pressure being understood through the Clausius-Clapeyron relation;

$${\int }_{{P}_{1}}^{{P}_{2}} d ln P = \frac{Δ {H}_{vap}}{R} {\int }_{{T}_{1}}^{{T}_{2}} \frac{1}{{T}^{2}} \cdot dT$$

$$ln \frac{{P}_{2}}{{P}_{3}} = \frac{Δ {H}_{vap}}{R} ( \frac{1}{{T}_{1}} - \frac{1}{{T}_{2}} )$$

wherein P denotes pressure, R the universal gas constant, ΔHᵥₐₚ the latent heat of vaporization and T the temperature in Kelvin. For example, acetone has a standard boiling point of 330K and ΔHᵥₐₚ=31 kJ/mol. Thus, even a roughing pump may provide for ˜−29 inHg gauge pressure and reduce the boiling point of acetone to ˜−12° C. It should be kept in mind, however, that reduced boiling points will cool the mixture. For example, placing the mixture in a heated water bath during mixing and evaporation may provide for enhanced evaporation of the thinning agent and allow for the mixture to be at least partially cured prior to forming the diffuser. Because they typically have greater densities than the media into which they're mixed, partial curing may serve to limit the sedimentation rate of scattering particles; a linear function of both the viscosity and particle diameter, and provide for easier handling prior to final curing.

The mixture may then be finally cured in a micromold, or in a tube whose ID matches that of the applicator in which it will ultimately be employed.

μₛ is a linear function of the volume fraction of scattering particles, fᵥ. Thus, altering fᵥ for a specific scattering particle may allow for tailoring a diffuser for the values of μₛ, or μ_{s′} desired. For example, the values of fᵥ in the embodiment of FIGS. 18A-18C may be adjusted to 0.15%, 0.26%, and 1% when using 0.5 μm microspheres (for which g=0.8 at 635 nm, as specified) in order to obtain the specified values of μₛ described as sections 102, 104, and 106 in the example of FIG. 17, respectively.

In a further embodiment, a tailored-refractive-index polymer (TRIP) may be used as an embedding material. A TRIP is a polymer that has a refractive index which is an amalgamation of its constituent ingredients. The refractive index is related to the molar refractivity, structure and weight of the monomer. In general, high molar refractivity and low molar volumes increase the refractive index of the polymer. Sulfur-containing substituents including linear thioether and sulfone, cyclic thiophene, thiadiazole and thianthrene are the most commonly used groups for increasing refractive index of a polymer in forming a TRIP. Polymers with sulfur-rich thianthrene and tetrathiaanthrene moieties exhibit n values above 1.72, depending on the degree of molecular packing. Phosphorus-containing groups, such as phosphonates and phosphazenes, often exhibit high molar refractivity and optical transmittance in the visible light region. Polyphosphonates have high refractive indices due to the phosphorus moiety even if they have chemical structures analogous to polycarbonates. In addition, polyphosphonates exhibit good thermal stability and optical transparency. Organometallic components also result in TRIPs with good film forming ability and relatively low optical dispersion. Polyferrocenylsilanes and polyferrocenes containing phosphorus spacers and phenyl side chains show unusually high n values (n=1.74 and n=1.72), as well, and are also candidates for waveguides.

Hybrid techniques which combine an organic polymer matrix with highly refractive inorganic nanoparticles may be employed to produce polymers with high n values. The factors affecting the refractive index of a TRIP nanocomposite include the characteristics of the polymer matrix, nanoparticles, and the hybrid technology between inorganic and organic components. Linking inorganic and organic phases is also achieved using covalent bonds. One such example of hybrid technology is the use of special bifunctional molecules, such as 3-Methacryloxypropyltrimethoxysilane (MEMO), which possess a polymerizable group as well as alkoxy groups. Such compounds are commercially available and can be used to obtain homogeneous hybrid materials with covalent links, either by simultaneous or subsequent polymerization reactions. The following relation estimates the refractive index of a nanocomposite,

n_{comp}=ϕₚnₚ+ϕ_{org}n_{org}

where, n_{comp}, nₚ and n_{org} stand for the refractive indices of the nanocomposite, nanoparticle and organic matrix, respectively, while ϕₚ and ϕ_{org} represent the volume fractions of the nanoparticles and organic matrix, respectively. The nanoparticle load is also important in designing TRIP nanocomposites for optical applications, because excessive concentrations increase the optical loss and decrease the processability of the composites. The choice of nanoparticles is often influenced by their size and surface characteristics. Direct mixing of nanoparticles with the polymer matrix often results in the undesirable aggregation of nanoparticles that scatter light. This may be avoided by modifying their surface, or thinning the viscosity of the liquid polymer with a solvent such as xylene; which may later be removed by vacuum during ultrasonic mixing of the composite prior to curing. Nanoparticles for TRIPs may be chosen from the group consisting of: TiO₂ (anatase, n=2.45; rutile, n=2.70), ZrO₂ (n=2.10), amorphous silicon (n=4.23), PbS (n=4.20) and ZnS (n=2.36). Further materials are given in the table below. The resulting nanocomposites may exhibit a tunable refractive index range, per the above relation.

| Substance | n (413.3 nm) | n (619.9 nm) |
|---|---|---|
| Os | 4.05 | 3.98 |
| W | 3.35 | 3.60 |
| Si crystalline | 5.22 | 3.91 |
| Si amorphous | 4.38 | 4.23 |
| Ge | 4.08 | 5.59-5.64 |
| GaP | 4.08 | 3.33 |
| GaAs | 4.51 | 3.88 |
| InP | 4.40 | 3.55 |
| InAs | 3.20 | 4.00 |
| InSb | 3.37 | 4.19 |
| PbS | 3.88 | 4.29 |
| PbSe | 1.25-3.00 | 3.65-3.90 |
| PbTc | 1.0-1.8 | 6.40 |
| Ag | 0.17 | 0.13 |
| Au | 1.64 | 0.19 |
| Cu | 1.18 | 0.27 |

In one exemplary embodiment, a TRIP preparation based on PDMS and PbS, the volume fraction of particles needs to be around 0.2 or higher to yield n_{comp}≥1.96, which corresponds to a weight fraction of at least 0.8 (using the density of PbS of 7.50 g cm⁻³ and of PDMS of 1.35 g cm⁻³). The information given above allows for the recipe of other alternate formulations to be readily ascertained. There are many synthesis strategies for nanocomposites. Most of them can be grouped into three different types. There are multiple preparation methods. Any and all of them may be employed to practice the present invention. Some are based on liquid particle dispersions, and may differ in the type of the continuous phase utilized. In melt processing particles may be dispersed into a polymer melt and nanocomposites obtained by extrusion. Particle dispersions in monomers and subsequent in-situ polymerization may also be employed. Although this discussion was centered around high refractive index composites, low refractive index composite materials may also be prepared in the same way. As suitable filler materials, metals with low refractive indices below 1, such as gold (shown in the table above) may be chosen, and the resulting low index material used to create a relatively negative refraction.

3. Diverters:

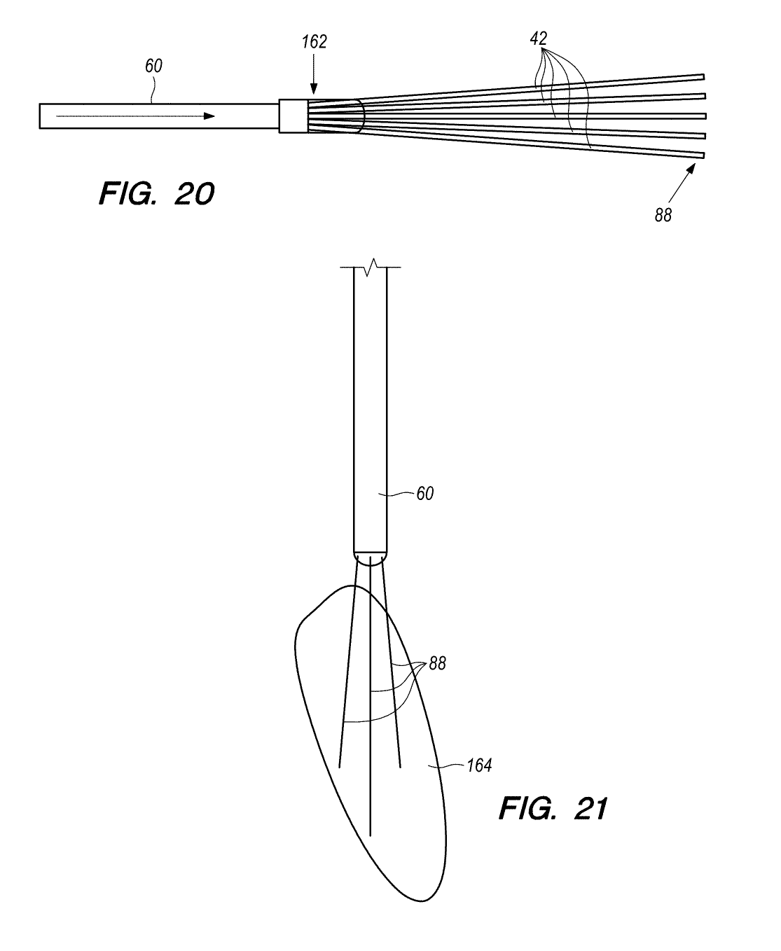

A diffuser may be attached to the distal end of a delivery fiber to form a probe. Probe body 60 may be configured to utilize diverter 162 (or “deflector”), such as that illustrated in FIG. 20, may be needed to maintain a lateral separation of the diffusers 88 and is shown in the analysis of FIG. 12. The diverter may also be used to deploy the fibers with the diffusers once the probe is placed in the tissue. The separation of the diffusers may be determined by trading-off the desired (suprathreshold) illumination volume against peak fluence rate level, as described elsewhere herein. The closer together the diffusers, the greater the increase of the fluence rate between fibers by their mutual illumination contributions may be. Likewise, the farther they are spread, the larger the volume may be. However, at too large of a distance the individual light fields emitted by each diffuser may become separate and thereby leave gaps in the regions between fibers that fall below the opsin activation threshold level that result in untreated portions of the target tissue.

Alternately, a probe may be constructed such that the applicators do not form a symmetrical pattern. Such a configuration may be useful, for example, when a target is itself asymmetrical, such as the STN, or when the target presents obliquely, or off-axis due to the surgical access route.

FIG. 21 illustrates an alternate embodiment configured for use with an asymmetrical target. In this exemplary embodiment, target 164 may be non-regular shape, as is often found in anatomy, and diffusers 88 (applicators) of probe body 60 may be configured to differing lengths, and/or diverter angles to accommodate the idiosyncrasies of the target. For example, the STN is generally biconvex-shaped structure that resembles a lens, or lenticule. Surgical access to the STN in humans may be made in a parasagittal plane moving rostral to caudal at an angle of 70° to the orbitomeatal line. In this configuration, the STN may present to the probe as an oblique oblate ellipsoid. Thus, the applicators may have a degree of symmetry, but not be completely symmetric (e.g. not radially symmetric about a center applicator).

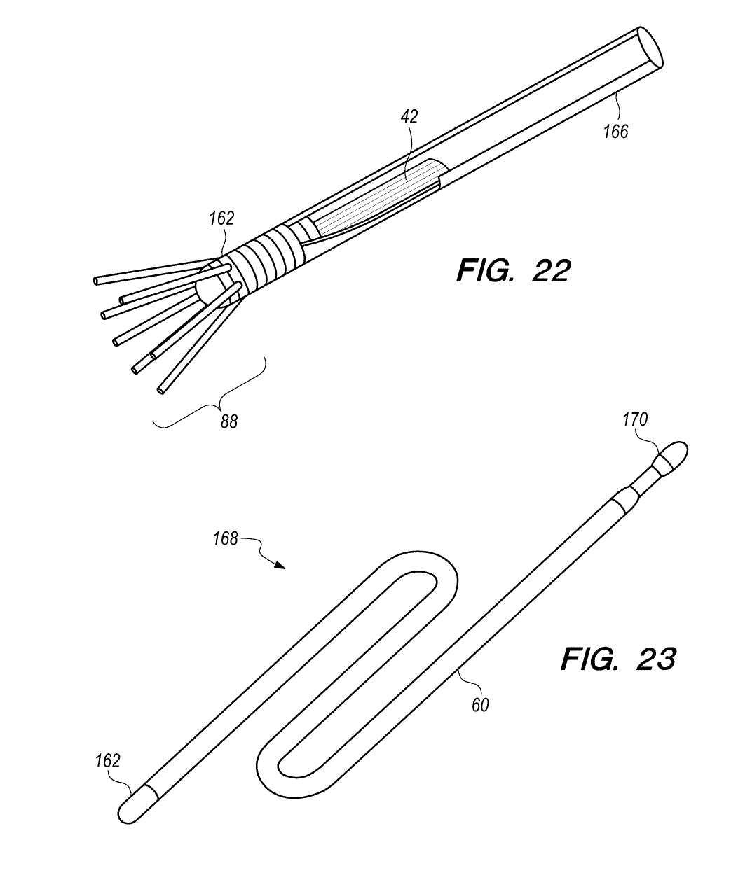

FIG. 22 illustrates a partially cut-away view of an embodiment of a probe body 60 further configured to utilize a diverter 162 to spread diffusers 88 into a region of target tissue 164 (not shown) and provide a fiber separation distance, as described above. Such a configuration may provide the benefits of minimizing the size of the implant and an enhanced illumination volume. In this example, a plurality of optical fibers 42 that each feed an individual diffuser 88 may be enclosed within a probe body that in turn may be covered with a biocompatible polymeric outer jacket 166 (such as polyurethane, for example) to prevent cellular ingrowth and contamination within the probe that may make its later removal more difficult.

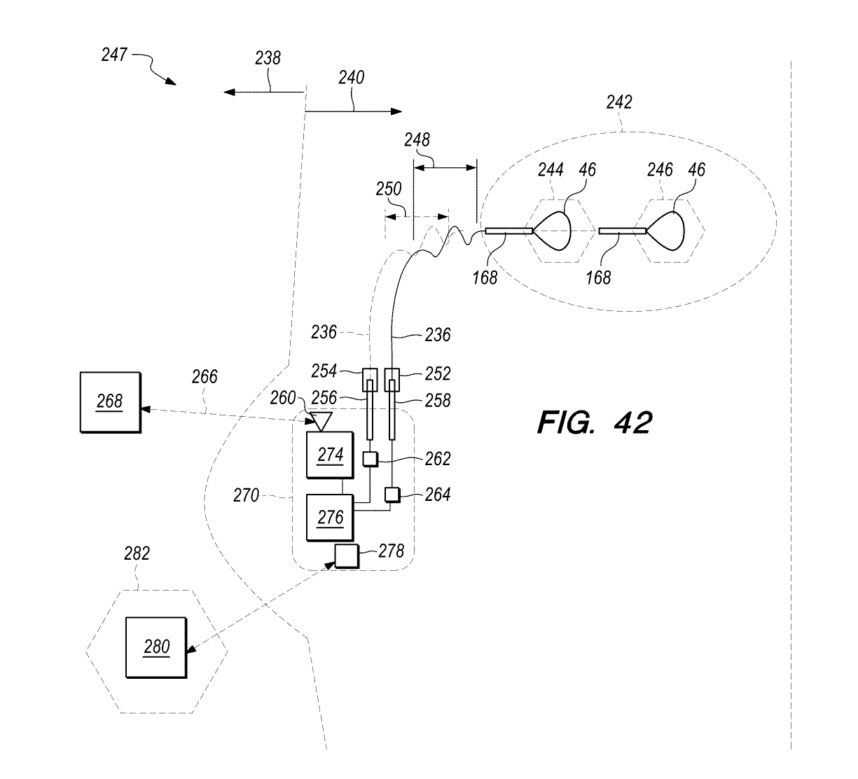

FIG. 23 illustrates a further embodiment, where a probe 168 is displayed in its entirety, including a proximal connector 170 that may be used to couple the probe to a trunk cable, or light source to form a system, such as that described with respect to FIG. 42 and also in U.S. patent application Ser. Nos. 14/737,445 and 14/737,446, both entitled, “Optogenetic Therapies for Movement Disorders”, which are incorporated by reference herein in their entireties, diverter housing 162 and a flexible probe body 60.

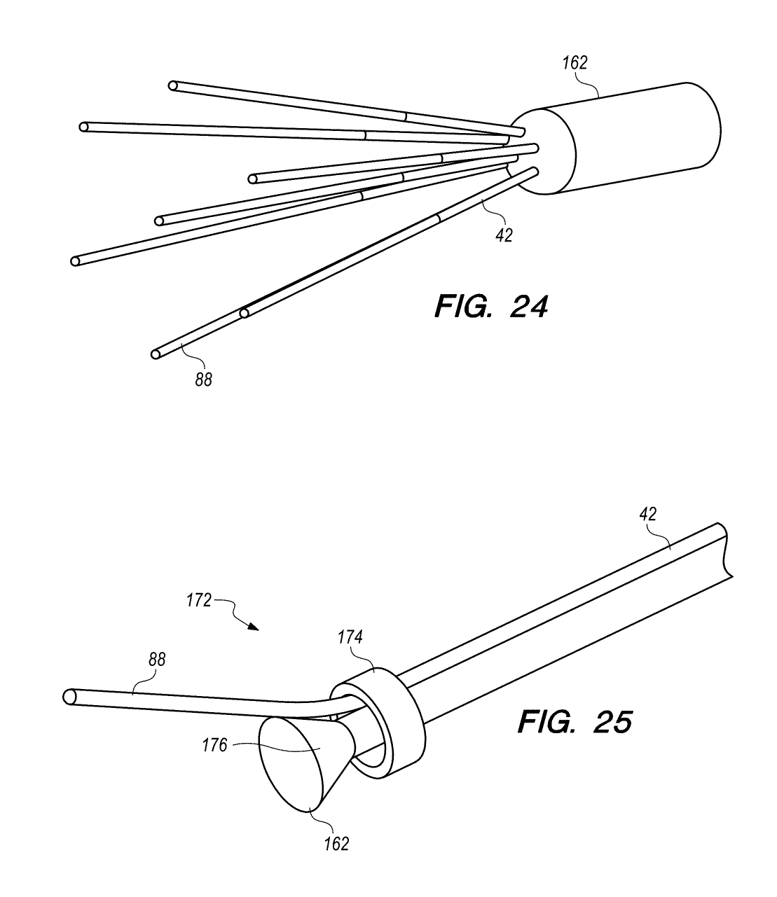

FIG. 24 illustrates a further embodiment, where more details regarding the deployment of diffusers 88 is shown as a fiber-to-diffuser configuration similar to that of FIGS. 6A-6B and FIG. 9A through FIG. 17. Light is conveyed to diffusers 88 by fibers 42, all of which may be advanced through diverter housing 162 to create a pattern of diffusers in target tissue.

The fiber optics may be contained in a blunt-nosed, tubular probe that is similar in materials, size and flexibility to those used for deep brain stimulation (DBS). For consistency with earlier examples and embodiments, a radially symmetric 7-fiber hexagonal configuration is shown in various figures herein, although other configurations and fiber numbers are considered within the scope of the present invention. The trajectory of the individual fibers may be defined by a diverter that directs the fibers as they are extended out of the probe tip (advanced) and into the target area.

Diagram 172 of FIG. 25 illustrates the basic concepts of a diverter, comprising a single optical fiber 42 terminating in a diffuser 88, a diverter 162, a guide surface 176, and a containment ring 174 surrounding at least partially fiber 42 and guide surface 176. Guide surface 176 and containment ring 174 constrain optical fiber 42 and/or diffuser 88 to deflect, or deviate from an otherwise nominally straight trajectory. That is to redirect the path of the optical fiber as it advances. It may be seen that a radially distributed plurality of such fibers in this exemplary configuration would nominally define a cone, as was shown in relation to the examples of FIGS. 20-22 and FIG. 24.

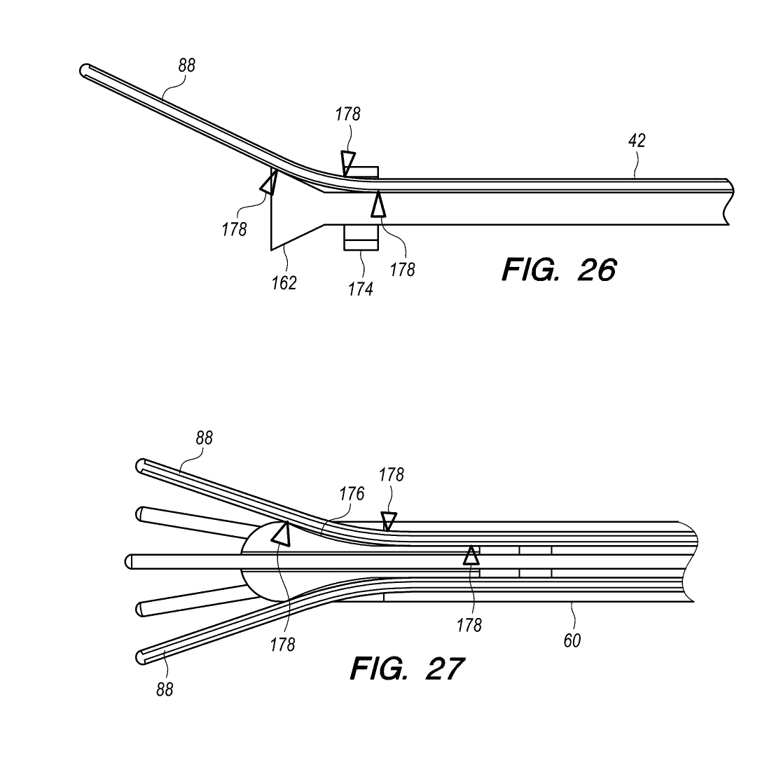

FIG. 26 expands upon the exemplary diverter shown in FIG. 25, with the added detail of contact points 178. Contact points 178 may define the deflection angle and radius of curvature of optical fiber 42. The contact points may also be surfaces, regions, or lines of contact to accomplish the same effect; as is shown as guiding surface 176 in FIG. 27, which may form at least a partial channel to guide optical fiber 42 and/or diffuser 88.

In a further embodiment, more than one diverter may be used to constrain the angular relationship between applicators. For example, a first diverter may be used to spread the fibers, and a second larger diverter may be used in a reverse orientation to “collimate” the optical fibers and/or applicators. This may be done to limit the bulk of the probe, only increasing its width or diameter in a distal region.

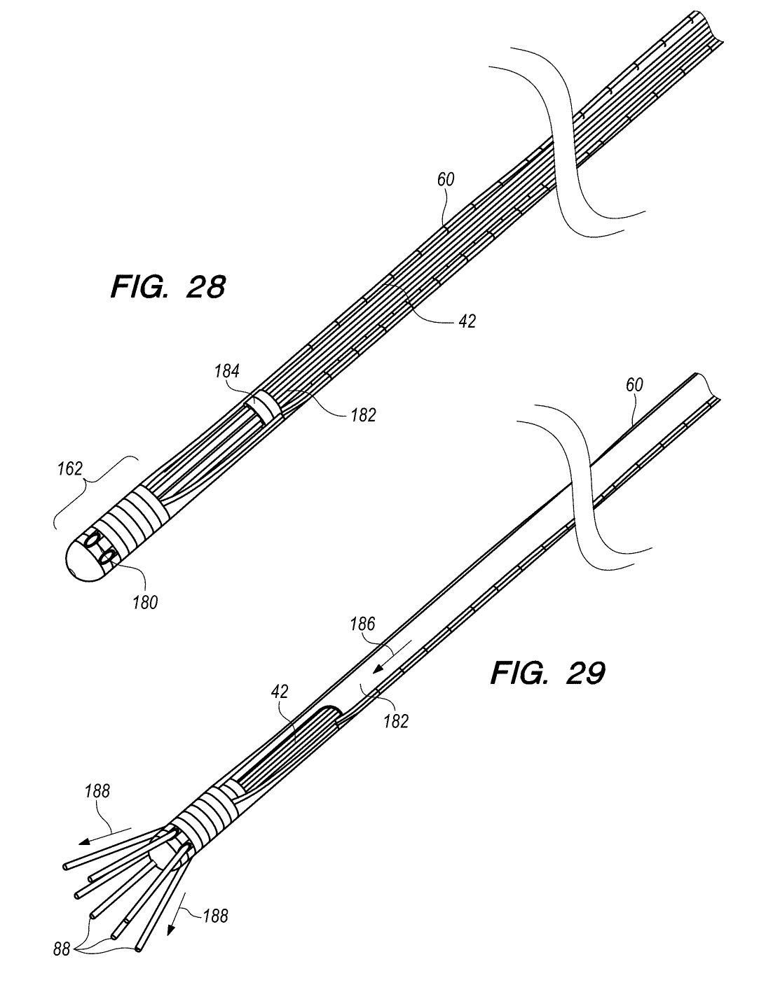

FIG. 28 illustrates an alternate embodiment of an integrated probe assembly 168, comprising probe body 60 with optical fibers 42 contained therein, a diverter tip 162 further comprising fiber ports 180 therein, and collar 184 that is attached to the optical fibers and/or the applicators. Collar 184 may be advanced distally to push the applicators into the target tissue by means of ejector 182. Ejector 182 may be a push rod, a sheath inside of the probe body, for example. A probe body 60 may be comprised of a polymer tube, an elastomer tube, a metal tube, a coil/spring, or a combination of thereof, for example. By way of nonlimiting example, a metal tube may comprise a cut in its wall to increase its flexibility, like an interrupted helical cut, for example. By way of nonlimiting example, a probe body 60 with a cut in its outer surface may be further configured to comprise a coating or cover 166 to provide a barrier and thereby reduce the open area that may allow for ingress of fluids and infiltrates. By way of nonlimiting examples, an exterior sheath or covering 166 may be chosen from the list containing; a conformal coating, a polymer coating, an elastomer coating, a silicone coating, a parylene coating, and a hydrophobic coating. The diverter tip may, for example, be fabricated from metal, such as stainless steel, or a polymer, such as PTFE, using screw machining techniques.

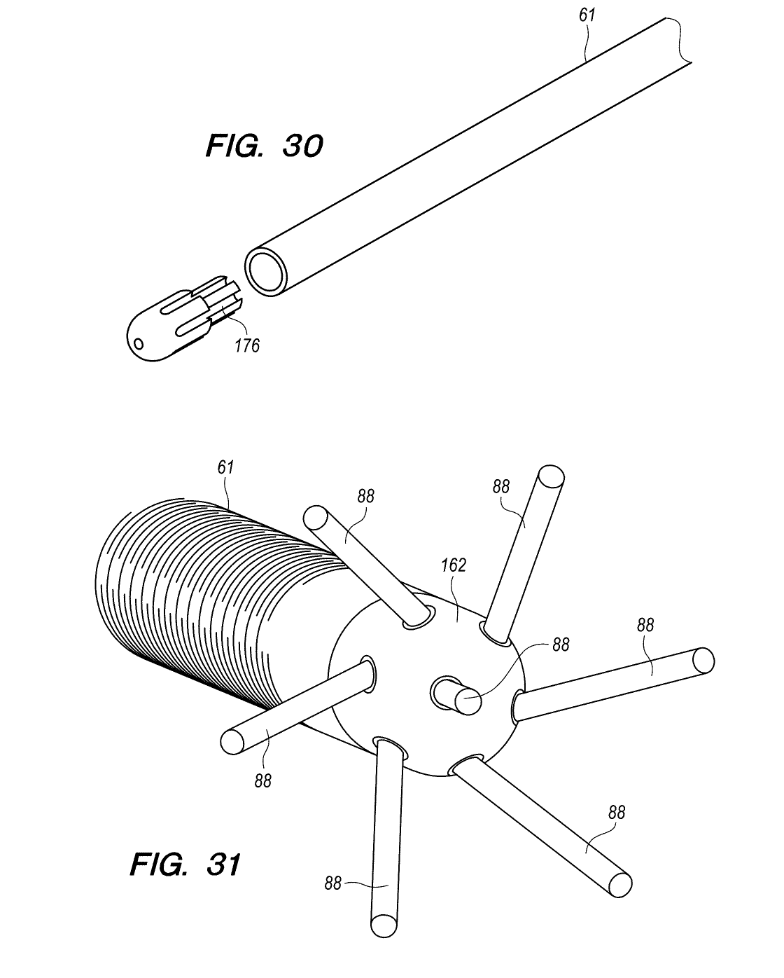

FIG. 29 illustrates an alternate embodiment of the probe of FIG. 28, with the additions of the ejector being an inner sheath that has been advanced in direction 186 to advance diffusers 88 in deployment direction 188, as indicated by arrows.

FIG. 30 illustrates an alternate embodiment, similar to that of FIGS. 28 and 29, wherein the diverter tip contains guide surfaces 176 and probe body 61 further comprises a subsumed containment ring 174 within the probe body itself.

FIG. 31 shows further embodiment, similar to that of FIG. 30 with the addition of guiding surfaces 176 (channels) made at compound angles to provide non-radial deflection which may allow for similar deflection angles but reduced radii of curvature than so-called radial deflectors by utilizing a larger volume of the diverter tip to create the deflection guiding surface/channels than may be available using a radial-diverter.

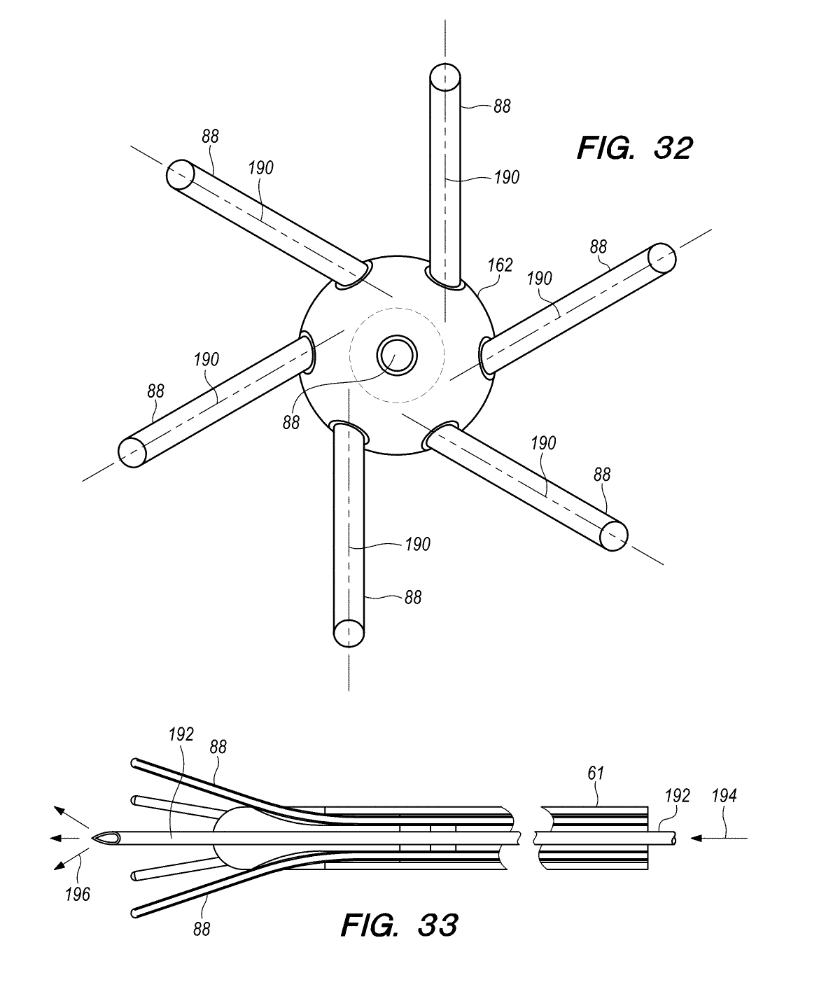

FIG. 32 shows an alternate view of the exemplary probe of FIG. 31, wherein the centerlines of diffusers 88 and guiding surfaces 176 are shown as dashed lines 190 to better indicate the path travelled in such a non-radial diverter.

A probe may be inserted into the brain of a patient using the same stereotaxic surgical apparatus akin to that used to implant a DBS probe. Furthermore, a removable infusion cannula may be incorporated into the probe, and the same probe may be used to both administer the gene therapy compound and illuminate the target tissue.

FIG. 33 shows a further embodiment, with the addition of an infusion cannula 192 that may occupy, at least temporarily, a central lumen of probe 168. In this way, the same target tissue 164 may be accessed with a single probe insertion for both delivery of a gene therapy agent and, later for tissue illumination. Arrows 196 indicate the direction of infusion of infusate 194.

Alternately, the applicators may be configured in pre-set bends that serve, at least partially, to create separation between applicators. For example, a tube may be used to contain the applicator and/or optical fiber, with the tube further comprising a thermally induced shape set that may be made by heating the tube and/or the tube/applicator assembly to a temperature sufficient for plastic deformation. The shape-set applicator may be then positioned inside the probe body through a diverter tip, or at least a fiber port and deployed, say using an ejector, as described elsewhere herein. By way of nonlimiting examples, the tube for shape-setting may be selected from the group consisting of; PEEK, Polyurethane, Tecothane, and PVDF. The shape may be set into the tube by placing it in a preconfigured channel that is cut into a block, or a pair of matched blocks that is/are then controllably heated to a temperature that renders the plastic pliable, nominally <the material glass temperature, then cooling it in place to set the shape. For example, PEEK 381G tubing may be placed in a pair of mating aluminum blocks, each containing a channel that consists of a straight section and curved section comprising 20° of a 10 mm radius of curvature. The channel may be cut using a ball endmill that is nominally only slightly larger than the outer diameter of the tubing. The blocks may be heated to from room temperature to a temperature of 85° C. over a period of 5-10 minutes and left to set for 30 seconds and then allowed to passively cool to room temperature in air over a period that is no shorter than 10 minutes to provide a uniform circular preset shape without undue residual stresses. Alternately, instead of polymeric compounds Nitonol may be used as sheath to provide a predefined shape to an applicator.

In a further alternate embodiment, a combination of pre-shaped applicators and a diverter may be used to provide nominally parallel applicator segments in tissue, as described above.

A constant curve, such as a circle or a line, may be preferable in order to avoid bisecting tissue during applicator deployment. In this manner, the applicator may follow a smooth, continuous path without lateral deviation.

Furthermore, a radio-opaque material may be used in the probe assembly to provide location information for intraoperative or postoperative imaging. Examples of radio-opaque materials are BaSO₄, metals and RO PEEK tubing, as is sold by Zeus, Inc., for use with applicators.

4. Skull Anchors:

The Skull Anchor may be attached directly to the skull and may pass through the burr hole that is created during treatment. It may serve to retain the intracerebral probe in its proper location relative to the brain region being treated (e.g. the STN). Its design may help the probe maintain a gentle bend as the probe traverses the skull. It may be made of medical grade polymers and affixed to the skull using stainless steel or titanium bone screws.

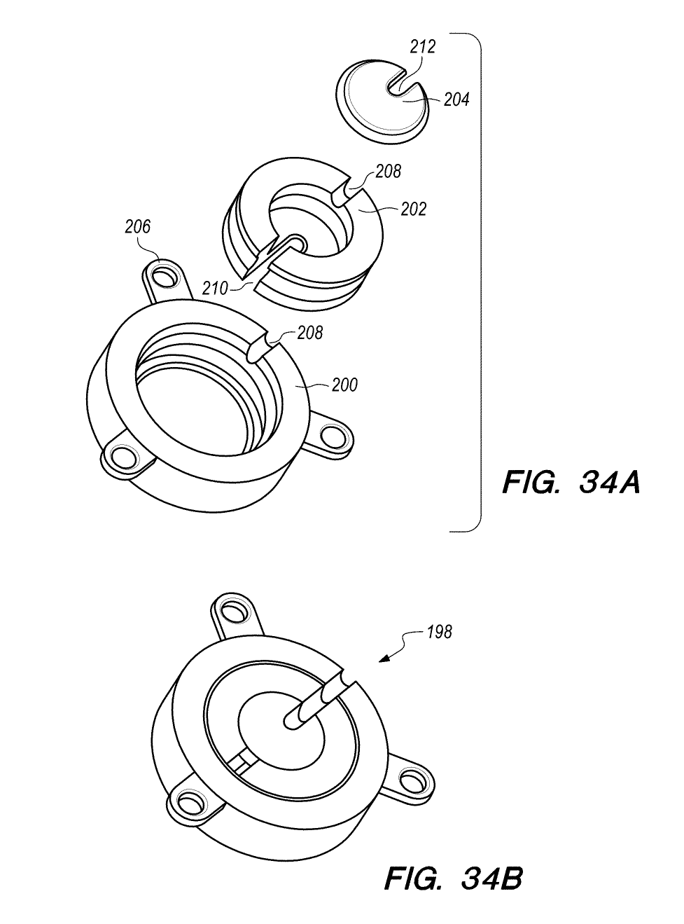

FIGS. 34A and 34B illustrate an example of a further embodiment, wherein a skull anchor assembly 198 comprises cap 204, collet 202, and ring 200. Ring 200 may be sized to fit at least partially within a burr hole in the skull and affixed to the skull using screws and mounting tabs. A probe 168 may be routed through the ring first once affixed to the skull using mounting tab 206, then collet 202 may be inserted into ring 200 and aligned such that the probe body 60 or 61 lies in slot 210 and channels 208 are aligned to provide a fixed bend in probe 168. Lastly, cap 204 may be added to the assembly of ring 200 and collet 202 with slot 212 aligned with channels 208 to provide a secure path to hold the probe in place. The height of mounting tabs 206 may be made to be about equal to the outer diameter of the probe body 60 or 61 to minimize discontinuities between anchor 198 and skull surface, as probe 168 may be made to lay atop and be routed along the skull.

Alternately, an aspect of skull anchor 198 may be to maintain a minimum bend radius for the optical fibers within probe 168 by direct contact of probe body 60 on at least a surface of anchor 198, particularly within collet 202 to form a continuous channel 208 to support probe 168. In a further alternate embodiment, the skull itself may be modified to act in concert with skull anchor 198 to route and/or maintain probe 168 in place and in a desired shape or pattern. Such skull modifications may include routing channels into its surface.

In an alternate embodiment, an outer surface of cap 204 and an inner surface collet 202 may be further configured to comprise locking elements between them. Similarly, an outer surface of collet 202 and an inner surface of ring 200 may be further configured to comprise locking elements between them. Exemplary locking elements are indentations and/or detents.

Exemplary materials for creating skull anchor 198 and its constituent components ring 200, collet 202 and cap 204 may be selected from the list comprised of; steel, stainless steel, PEEK, PMMA, PTFE, PET, and PETG. The skull anchor 198 may further provide for a minimum bend radius to be maintained for a probe 168. The minimum bend radius may be defined by a condition of the fibers 42 contained by a probe 168, as a means to mitigate optical losses and optical fiber failure due to stresses associated with tight bend radii. By way of nonlimiting example, a minimum bend radius may be about 4 mm for a probe 168 comprising optical fibers 42 that are about 110 μm in outer diameter. Furthermore, a minimum bend radius may be defined by a surface within a skull anchor assembly 198. A minimum bend radius surface may be described by a curve that lies in a single plane, or a curve that lies outside of a single plane in a compound manner, such as, by way of nonlimiting example, a helix or a helical segment. The minimum bend radius surface may be comprised of a channel, a tunnel, a hole, or a combination of a channel and/or a tunnel and/or a hole, by way of nonlimiting example.

5. Multi-Fiber Connectors and Trunk Cables:

There are two main types of conventional fiber optic connectors: one type has zero degree polish angle and is called PC (physical contact) connector, the other type is called APC (angled physical contact) connector which typically has an 8 degree tilted polish angle at the fiber facet in order to minimize back reflection. Both typically have ferrules to contain the fiber ends. These conventional approaches are typically created using a single fiber per ferrule, and make up the overwhelming majority of fiber optic connections. There are connectors that support multiple fibers, but they have been designed predominantly for use in optical mid-plane networking devices where repeated mating cycles are required, and are too large, bulky and unwieldy for clinical use. These connectors, such as the “MT” standard arrange the fibers along a line and make use of v-groove or similar mounting schemes for the fibers, which cause them to be space-inefficient and unsuitable for use in clinical applications that involve the use of cannulae to deploy probes into tissue. The use a cannula for deploying a multi-fiber probe in tissue requires that the cannula be removed by pulling the probe through it, or using what is known as a “tear-away” sheath. Tear-away sheaths do not routinely provide the required stability and rigidity required for accurate and precise placement in tissues such as the brain. Existing multifiber connectors do not allow insertion through a rigid cannula whose ID is about the same as the OD of a probe. It is often required to minimize the probe diameter in order to avoid the trauma associated with excessive and otherwise unnecessary tissue displacement. Thus, multifiber optical connectors are needed to effectively practice photomedicine using implantable probes.

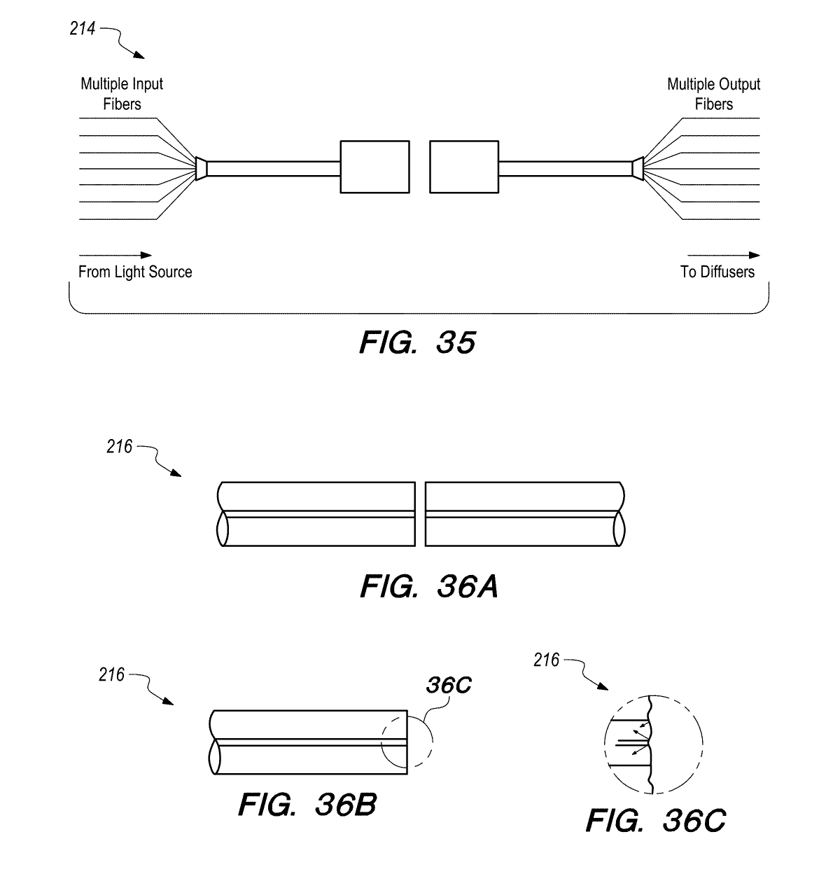

An example of the basics of a one to one connector for 7 fibers is shown in diagram 214 of FIG. 35. One purpose may be to transfer light efficiently from an input fiber to an applicator including a diffuser while enabling the ability for multiple cycles of connecting and disconnecting. A connector affords the ability of separating the light source subsystem from the applicator subsystem, which in turn provides the ability to separate the installation tasks for the applicator and light source, as well as to allow for diagnosis, and service capabilities such as subsystem replacement. The most important performance criterion may be to reduce insertion loss when connected. In this way, the highest transmission of light through the detector may be achieved. Other performance criteria may include return loss which is kept to a minimum and crosstalk between fibers which also is kept to a minimum. All these performance criteria may also be put into the context of other system considerations such as small size & usability.

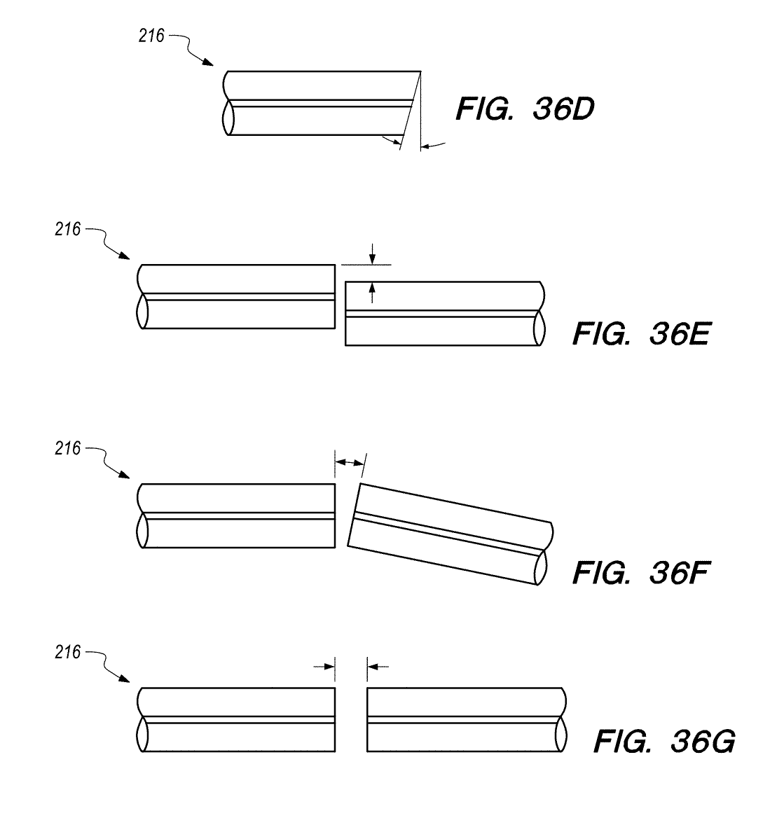

A multifiber connector may be a butt joint between two sets of fibers. Various diagrams 216 of FIG. 36 illustrate some typical issues associated with one to one butt coupling. These issues may be exacerbated by using multiple fibers in a single connector. Referring to FIG. 36A, end face losses related to reflection are illustrated. Referring to FIGS. 36B and 36C, end face losses related to surface quality issues, such as smoothness, are illustrated. Referring to FIG. 36D, end face losses related to end angle (flatness, perpendicularity) are illustrated. Referring to FIG. 36E, losses due to lateral offset (coaxiality) are illustrated. Referring to FIG. 36F, losses due to angular misalignment are illustrated. Referring to FIG. 36G, losses due to longitudinal distance (end gaps) are illustrated.

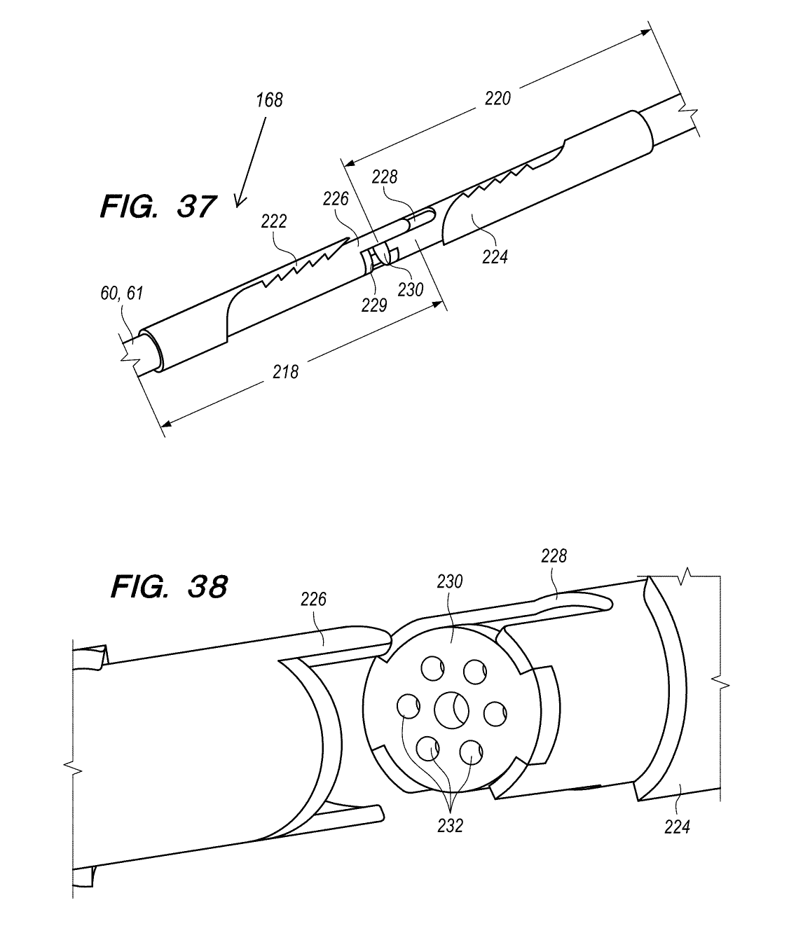

FIG. 37 shows an example of an embodiment of the present invention, a multi-fiber connector 218 is disposed on the proximal end of a probe 168, comprised of alignment feature 226, a tongue in this example, fiber face plate 229, and locking teeth 222. A second multi-fiber connector 220 is intended to be nominally complementary to multi-fiber connector 218, and comprises alignment groove 228 to engage alignment feature 226, locking teeth 224 to engage locking teeth 222 with the purpose of bringing fiber face plate 230 into contact with fiber face plate 229 of multi-fiber connector 218.

FIG. 38 shows a more detailed rendering of the multi-fiber connectors from FIG. 37, wherein the individual fiber ports 232 of multi-fiber connector 220 are resident on the ferrule face plate 230, and alignment tangs 226 may engage into alignment grooves 228 to provide rotational alignment, or clocking. Fibers are not shown for clarity. By way of nonlimiting example, a ferrule face plate may be fabricated from glass, ceramic, high density plastic, platinum and/or stainless steel, to provide a precision surface and to facilitate polishing of the fiber optics. Laser machining may provide for the required positional tolerances on fiber ports 232. Fiber ports 232 are intended to mate with complementary fiber ports on multi-fiber connector 218.

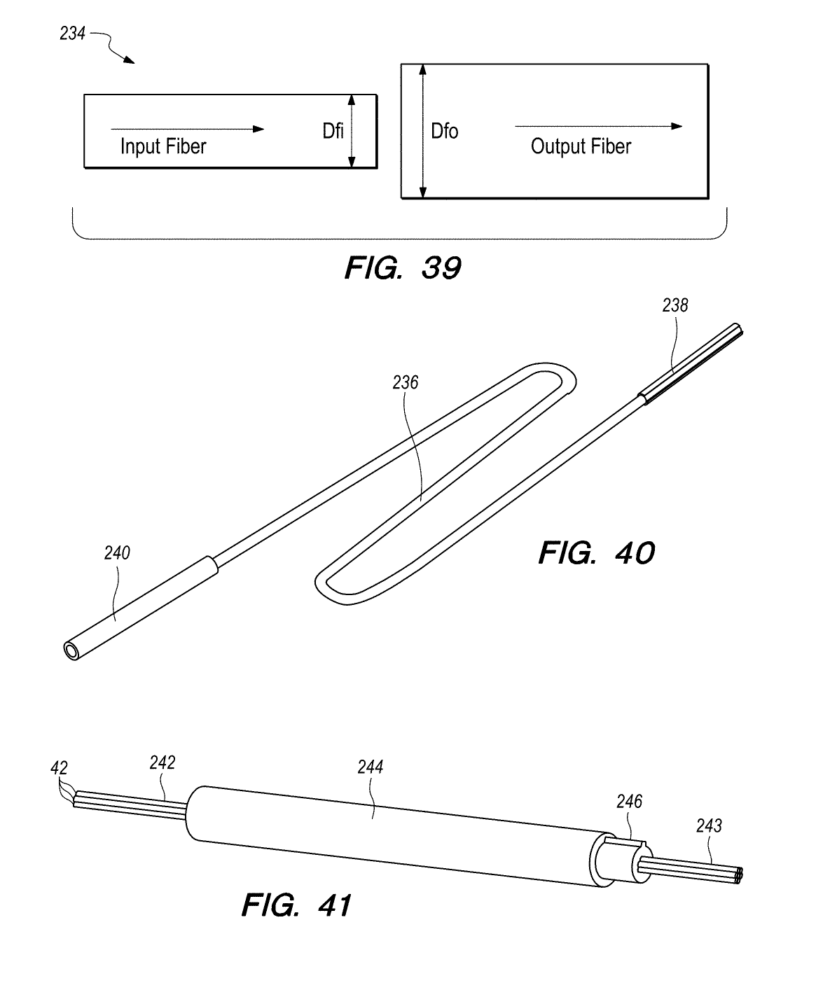

Coupling difficulties can be mitigated, at least in part, by stepping up the diameter of the output fiber relative to the input fiber. That is, increasing the diameter of a distal fiber to a proximal fiber for improved optical coupling. For example, the core of input fiber can have a diameter of 70 μm while the core of the output fiber 42 can be 100 μm. The cladding and/or buffer diameters may be nominally the same to help provide concentricity. Diagram 234 of FIG. 39 illustrates an example of this configuration, where the input (proximal) fiber has a smaller core diameter (Dfi) than that of the output (distal) fiber (Dfo), which represents fiber 42 as described in previous examples.

Additionally, the numerical aperture (NA) may also be increased in a similar manner to facilitate efficient coupling. For example, the input fiber can have an NA=0.12 while the output fiber can have an NA=0.22, and possible mitigate angular alignment sensitivities. The NA step-up may have less of an effect than the diameter step-up and matching NAs may be suitable. Combinations of proximal-to-distal diameter and NA increase are also possible.

An Extension Lead may couple the intracerebral probe to a light source. It may be included in an implanted medical device to simplify the implantation procedure and permit the implantable light source to be placed in a location more amenable to the planned size of the device, such as the anterior chest wall (infraclavicular) or abdomen. The extension lead may consist of a polyurethane tube, which may contain a plurality of fiber optics; terminated with two precision optical connectors that efficiently link the fibers of an intracerebral probe to the laser output from an implantable laser controller, such as is described elsewhere herein. Alternately, a short wire (such as Silver core MP35N) that may serve as an antenna for the MICS system may also be resident in the extension lead. Furthermore, they may be covered by a boot (made from polyurethane, PTFE, or other materials) to prevent fluid ingress and cellular infiltration that may degrade the performance of the system.

FIG. 40 illustrates an embodiment of an aspect of the present invention, an extension lead 236 (or trunk cable), as described elsewhere herein, comprising a female distal end connector 240 and a proximal end connector 238, but without the boot covering for clarity. Both connectors 238 and 240 may be multi-fiber connectors.

FIG. 41 illustrates an alternate embodiment, wherein an extension lead 236 is connected to a probe 168 using multi-fiber connectors 238 and 240, respectively, that are similar to those of FIG. 37, with the addition of a keyway to provide clocking (i.e. rotational alignment about the long axis of the lead, or “roll”). The keyway is comprised of female connector 244 and male connector 246 that are used to couple fibers 42 in fiber bundle 242 from a probe 168 to fibers of fiber bundle 243 of an extension 236.

6. System Configurations:

FIG. 42 illustrates an exemplary embodiment of system 247, configured for therapeutic use such as those described in U.S. patent application Ser. Nos. 14/737,445 and 14/737,446, both entitled, “Optogenetic Therapies for Movement Disorders”, both of which are incorporated by reference herein in their entireties. Distributed emission probes 168 have light delivered to via extension leads 236, respectively, to create light fields 46, within the target tissues 244 and 246 within brain 242. Light fields 46 may be configured to provide illumination of a target tissue within the fluence rate range of 0.01-100 mW/mm², and may be dependent upon one or more of the following factors; the specific opsin used, its distribution and/or concentration within the tissue, optical properties of the target tissue and/or adjacent tissue(s), toxicity limits, and the size of the target structure(s). Optical fibers 42 for conveying light within probe 168 to diffusers 88 may be, by way of nonlimiting example, 100 μm core diameter/110 μm cladding diameter/130 μm polyimide buffer coated 0.22NA step index fiber (such as SFS100/110/130T from FiberGuide) that may be affixed to a 125 μm POF diffuser 88 (such as SMPOF125 from Paradigm Optical), and the fiber-diffuser assembly enclosed in a 200 μm OD biocompatible polymer capillary tube (such as PETG tubing CTPG150-200-5 from Paradigm Optical), whose distal end 108 may be encapsulated with a biocompatible epoxy to minimize contact between the optical surfaces and fluids within the body and thus better maintain its optical function. An extension lead 236, may also be used to conduct light from light sources 262 and 264 to diffusers 88 utilizing multiple fiber connectors 218 and 220, not shown. Multi-fiber connectors 252 and 254 may be configured to operatively couple light to extensions 236 from light sources 262 and 264. Extensions 236 and/or probes 168 may further comprise Undulations 248 and 250, respectively, which may provide strain relief and be held by a skull anchor assembly 198 (not shown). Light Sources 262 and 264 may be configured to be a blue laser source, such as the NDA4116 from Nichia that produces up to 120 mW of 473 nm light with a slope efficiency of ˜1 W/A, or the NDB4216E from Nichia that produces up to 120 mW of 450 nm light with a slope efficiency of ˜1.5 W/A, which are suitable for use in optogenetic intervention using such opsins as ChR2, iC1C2, SwiChR, and/or iChR2, by way of non-limiting examples. The system contained within housing 270 that may be located within intracorporeal space 240 may further comprise a telemetry module 274 coupled to antenna 260 that via communication link 266 to communicate with clinician/patient programmer 268 located in extracorporeal space 238 for defining illumination parameters such as pulse duration, repetition rate, and duty cycle; a controller 276 and a recharging circuit 278 that may communicate with external charging device 280, which itself may be contained within a mounting device 282.

Furthermore, also presented herein are dosages of light capable of therapeutic function through activation of an opsin within a cell, and dosage ranges that may correspond to threshold fluence rates and/or pulsatile parameters for therapeutic utility at the low end and a peak fluence rate and/or pulsatile parameters that may correspond to damage limit at the upper end to provide a safety range. These ranges relate to instantaneous and averaged values of the fluence rate within a tissue, and the temporal parameters of their delivery, as is defined elsewhere herein. These dosages may be administered to the cells of a patient to cause treatment of a disease or symptom, most commonly, but not limited to a neurological disease or symptom.

The upper dosage limit that can be provided safely to the cell may be defined as the peak fluence rate that is less than a damage limit. Several examples of empirically determined ranges are given in subsequent sections. Alternately, a “dosage unit” may also be considered in a manner similar to that used in pharmacology, where the integrated treatment energy for a given treatment duration, such as a day, for example, is described along with the temporal parameters of its delivery, as is described in an earlier section herein.

Importantly, the presently described light dosage is not dependent on a particular device or means of providing the light to the patient's cells. The light can come from natural sources, such as the sun. Additionally, the light may come from chemiluminescence or bioluminescence provided along with the opsins, or otherwise in proximity to the transformed cells such as, but not limited to, luminopsins (Tung et al. Scientific Reports, 5, Article number 14366 (2015)) or Nanoluc-based luminescence (Yang et al. Nature Communications 7, Article number 13268 (2016)), each of which is incorporated by reference herein in its entirety.

The lower dosage limit that can be provided may be that which is the minimal amount that provides a clinical benefit to the patient. One of ordinary skill in the art may determine such levels using any clinical readout that is appropriate for the disease to be treated. Some non-limiting examples include symptomatic measurements such as patient questionnaires, outward symptom measurement such as motor response, or any other means of measuring a change in a patient's disease or symptom state. See, e.g. Clinical Trials in Neurology, Ravina et al., eds., Cambridge University Press, 2012, incorporated by reference herein in its entirety. Alternatively, indirect measurements such as a change in pharmacodynamic biomarkers can form the basis for a finding of clinical benefit.

A second possible means of defining the lower limit of function of an opsin is the lowest amount of light that results in a physiological response in the organism that the opsin was originally isolated from. In this way, it can be anticipated that at least some impact of the light delivered would be expected upon the neuron which has been transformed with the protein, given the evolutionary development of the opsin for some functions within the source organism.

Given the relatively low limit of safe exposure of neural tissue to light, reported herein there may be a necessary limit to those opsins that are not highly sensitive to light exposure for the contemplated uses.

As described in the examples below, one relatively sensitive opsin is SwiChR++(Berndt et al. 2016, Structural foundations of optogenetics: Determinants of channelrhodopsin ion selectivity. Proceedings of the National Academy of Sciences, 113(4), pp. 822-829, see also WO2015148974; each of which is incorporated by reference herein in its entirety). A second set of opsins are those described by Spudich et al. Natural light-gated anion channels: A family of microbial rhodopsins for advanced optogenetics, Science349(6248): 647-650; incorporated by reference herein in its entirety. Finally, it is anticipated that alterations in known opsins through mutational analysis will improve sensitivities such that their function will fall within the safety limits described herein. See, e.g. Mclssac et al., Recent advance in engineering microbial rhodopsins for optogenetics, Curr Opin Struct Biol, 2015 33:8-15; incorporated by reference herein in its entirety.

The methods and configurations described herein may be utilized in many therapeutic scenarios, such as to treat neural tissues pertinent to neurological disease states including but not limited to Parkinson's disease, essential tremor, non-Parkinson's cerebellar degenerative disease, Alzheimer's disease, non-Alzheimer dementia, dystonia, epilepsy, migraine, non-migraine headache, trigeminal neuralgia, Guillain-Barre syndrome, Huntington's disease, myasthenia gravis, ataxia, narcolepsy, amnesia, transient global amnesia, cerebellar disease, and pain.

### Example 1—Effects of Long Term Illumination of Neural Tissue

One clinical application of optogenetic therapy for Parkinson's Disease (PD) patients is anticipated to be continuous modulation of the subthalamic nucleus (STN), similar to deep brain stimulation (DBS). Thus, we conducted a pilot study to determine whether the therapeutic effect continues for periods what had been tested previously in the art, typically 90 minutes. In this pilot study, 60HDA lesioned mice infused with AAV.hsyn-Arch-eYFP (n=4), as previously described (Duty, S. & Jenner, P., 2011. Animal models of Parkinson's disease: a source of novel treatments and clues to the cause of the disease. British journal of pharmacology, 164(4), pp. 1357-1391; Chow et al., 2010. High-performance genetically targetable optical neural silencing by light-driven proton pumps, Nature, 463:98-102; and Kügler S, Kilic E, & Bähr M, 2003. Human synapsin 1 gene promoter confers highly neuron-specific long-term transgene expression from an adenoviral vector in the adult rat brain depending on the transduced area. Gene Ther. 10(4), pp. 337-47; each of which is incorporated by reference herein in its entirety). The lesioned, transformed mice were fitted with a laser fiber tip that was positioned approximately 0.5 mm above the STN and were treated with 24 hours of continuous illumination. Animals were acclimated to the open field testing chamber containing bedding material, food and gel packs for 33 hours prior to the 24-hour presentation of illumination and an additional 14 hours post illumination. The normal 12 h light/12 h dark cycle was maintained during the entire experiment. For opsin activation, we used an illumination pattern that produced a therapeutic effect of 2 ms pulses at 250 Hz with peak intensity of 10 mW at 635 nm, which was equivalent to approximately 5 mW average power in the brain over the 24-hour period, resulting in a peak fluence rate of 415 mW/mm², and an illumination volume of 13.7 mm² for levels ≥1 mW/mm2. The Examples within the present application therefore provide data regarding extended continuous illumination in neural tissue at levels that can activate opsins and are therapeutically relevant.

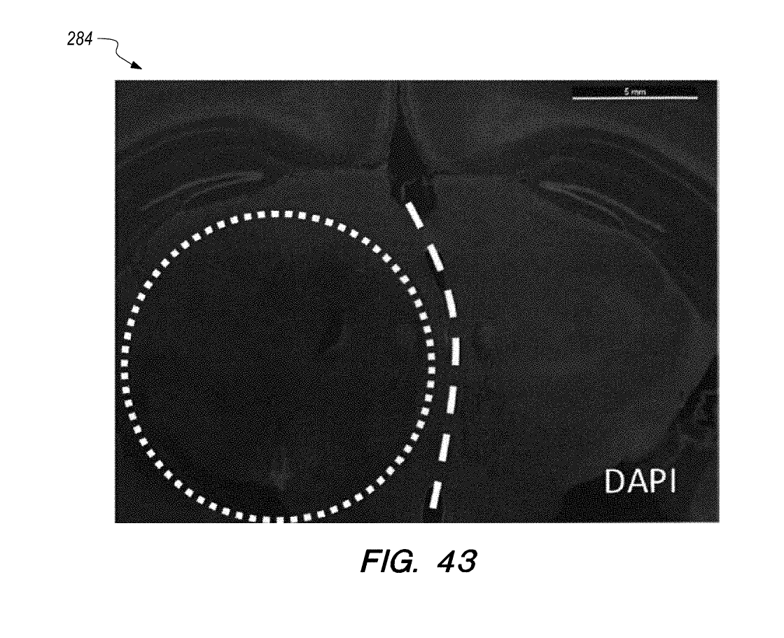

We discovered that this continuous 24-hour illumination treatment paradigm produced adverse consequences. Our behavioral observations indicated that there was a measurable rotational effect in the therapeutic direction for at least 2 hours, and by hour 10 of the continuous illumination, the animals exhibited hyperlocomotion, which might be consistent with therapeutic efficacy. However, by hour 15, the animals shifted back to tight rotation towards the side of injury, opposite of a therapeutic effect, and became immobile by hour 18 of illumination. Physical assessment of the animals after the 24 h illumination treatment showed that mice also had increased muscle tone contralateral to the side of treatment. Despite a 14-hour observation period after the illumination treatment ended, there was no reversal of the immobility, and a progressive deterioration in the overall status of these animals. Post-mortem histological analysis with a DAPI counter stain revealed massive brain edema and loss of brain cells (FIG. 43). Image 284 of FIG. 43 illustrates a micrograph of a DAPI stained mouse brain section, documenting neuronal loss resulting from optogenetic treatment within the large dotted line circle. We measured the area affected by of cell loss in the medial to lateral direction and anterior to posterior direction to determine the extent of damage caused by the illumination treatment, and found it to cover a large portion of the mouse brain of approximately 38±8 mm³. For this and all subsequent Examples, we used this post-mortem histological analysis of cell loss as an end-point measure of the illumination treatments, and deemed is safe only if there was no observable loss of cells, as assessed by DAPI counter staining.

### Example 2—Elimination of Opsin or Lesion Model Effect

Further investigation confirmed that the toxicity was not associated with the expression of the opsin Arch or the 60HDA lesion model, but was the result of the extended illumination of brain tissue. The same brain damage was observed in WT mice (n=16) that did not receive the 60HDA lesion and did not receive infusion of AVV to express Arch following 24 h illumination with 2 ms pulses at 250 Hz, with peak intensity of 10 mW at 635 nm (approximately 5 mW average power over the illumination period), resulting in a peak fluence rate of 415 mW/mm², and an illumination volume of 13.7 mm³ for levels ≥1 mW/mm2. We also discovered that this damage from extended illumination could be induced in different regions of the brain. In addition to the STN, which is a deep structure, we tested the thalamus (n=2), a midbrain structure, and motor cortex (n=3), a more superficial structure and observed the same type of damage. The damage was also not species specific, because we were able to replicate the results when testing for 24 hours of illumination in the brains of rats (n=4).

### Example 3—Investigation into Heat Contribution

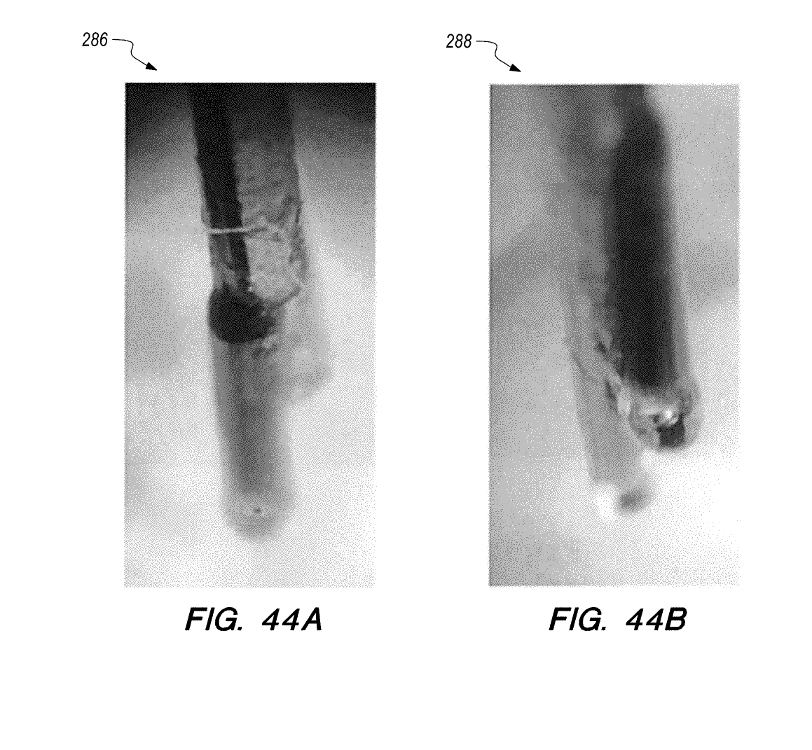

Traditionally, one key concern of continual illumination treatment in brain tissue has been the absorption and conversion of photons into heat. Thus, we sought to understand whether the therapeutically relevant illumination parameters were generating sufficient heat to explain the toxicity. We designed special light probes, consisting of a 200 um optic fiber attached to a thermistor that could record the average rise in temperature of the brain tissue around optic fiber during the illumination (FIGS. 44A and 44B). Image 286 of FIG. 44A is a photograph of a thermistor attached to front of the fiber delivering laser light utilized to investigate the role of heat in the observed results. Image 288 of FIG. 44B is a photograph of a thermistor attached behind the fiber delivering laser light utilized to investigate the role of heat in the observed results. Thermistors were individually calibrated and specific Steinhart-Hart coefficients were calculated and used to provide accurate temperature readings that were validated using a NIST-traceable thermocouple. Using these probes we measured the immediate change in temperature and found an increase of ˜0.6° C. (n=192 measurements across 3 pigs and another n=15 measurements across 5 mice) when using the same illumination parameters for which we saw extensive toxicity in the brain (see Example 1). This nominal temperature rise is well below the consensus standard limit of 2° C. and not commonly thought to cause tissue damage.

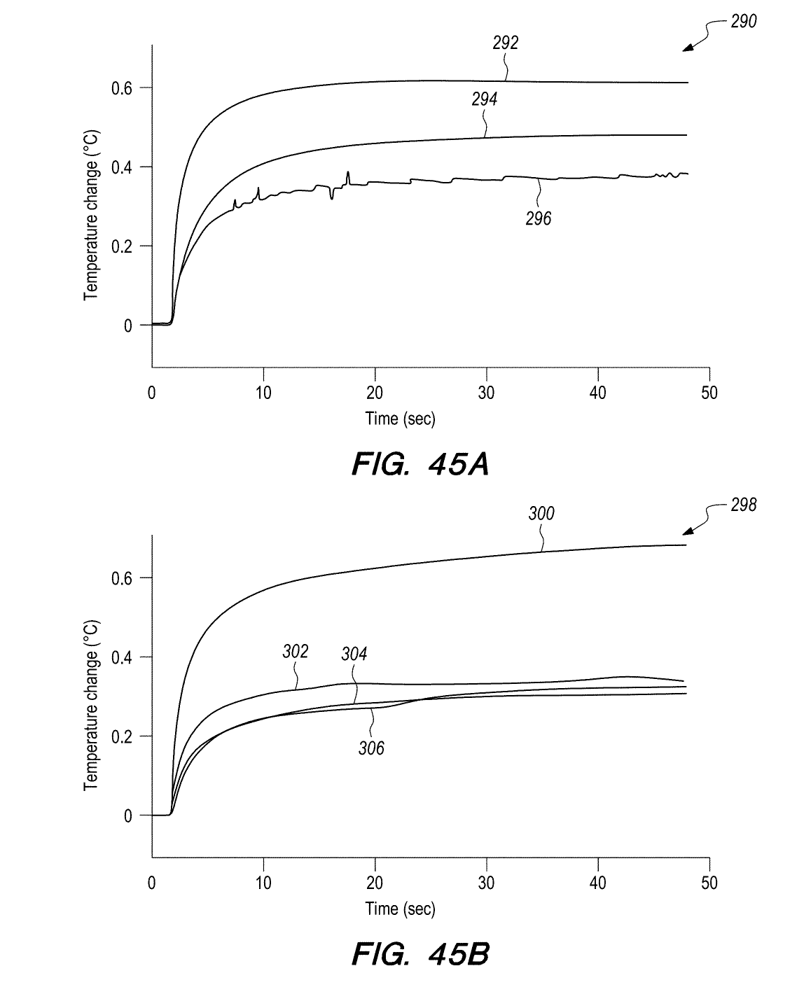

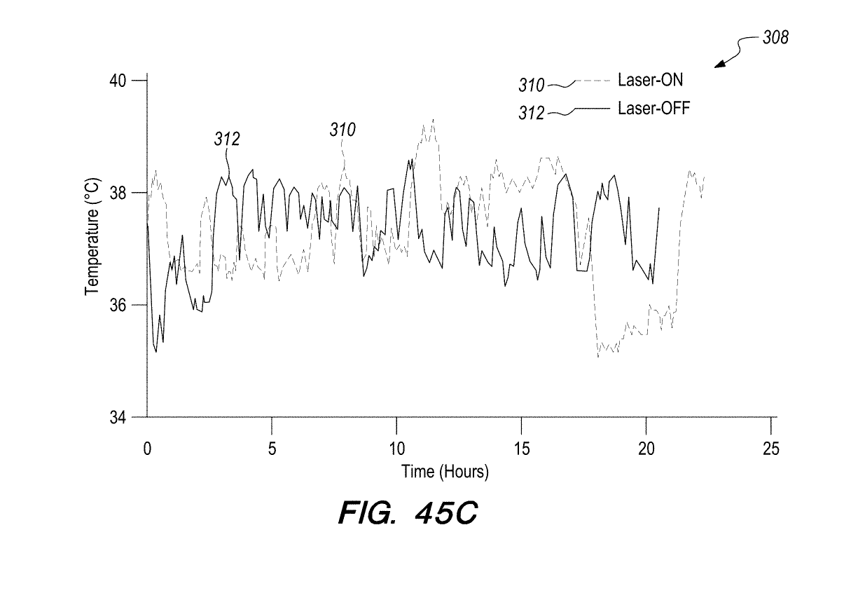

Plot 290 of FIG. 45A plots the short-term temperature change over time in an anesthetized mouse, comprising data 292, 294, and 296 representing measurements for different delivered optical powers. Plot 298 of FIG. 45B plots the short-term temperature change over time in an awake mouse, comprising data 300, 302, 304, and 306 representing measurements for different delivered optical powers. These results agree well with the predicted temperature rise, given by the following relation

$$Δ T = \frac{{P}_{a}}{4 πα r} erfc ( \frac{r}{\sqrt{4 α t}} )$$

$${P}_{a} = {μ}_{a} ( λ ) P ( r , θ , φ )$$

wherein α denotes the thermal diffusivity of tissue, r the distance from the heat source, t the time, Pₐ the absorbed optical power, μₐ the optical absorption coefficient, and P the optical power distribution.

Plot 308 of FIG. 45C shows a comparison of the temperature changes with the laser on (trace 310) and with the laser off (trace 312) over 22 hours of therapy. The maximal immediate increase in temperature was similar in both anesthetized and awake mice, with recorded temperature rises tending to be smaller in the awake condition (compare FIG. 45A (anesthetized) and FIG. 45B (awake)), suggesting greater heat dissipation in the awake mice. This was further corroborated by continuous monitoring of changes in temperature of the brain during the extended illumination of over 22 hours (FIG. 45C). Here, the brain tissue temperature was monitored for 20 hours in the absence of illumination to determine a baseline (FIG. 45C, trace 312) and compared to the 22+ hours of illumination (FIG. 45C, trace 310) at the same parameters (see above) that produced extensive toxicity in brain tissue. The fluctuation in average brain temperature was indistinguishable between the two conditions. Furthermore, there was no apparent accumulation of heat, with the temperature rise during with the temperature rise during the course of the 22+ hours of illumination. This suggests that, overall, mice are able to compensate for the additional illumination-induced heat, and maintain the brain tissue temperature in the same physiological range, which is inconsistent with heat being the root cause of the toxicity.

### Example 4—Testing of Heat Contribution without Light

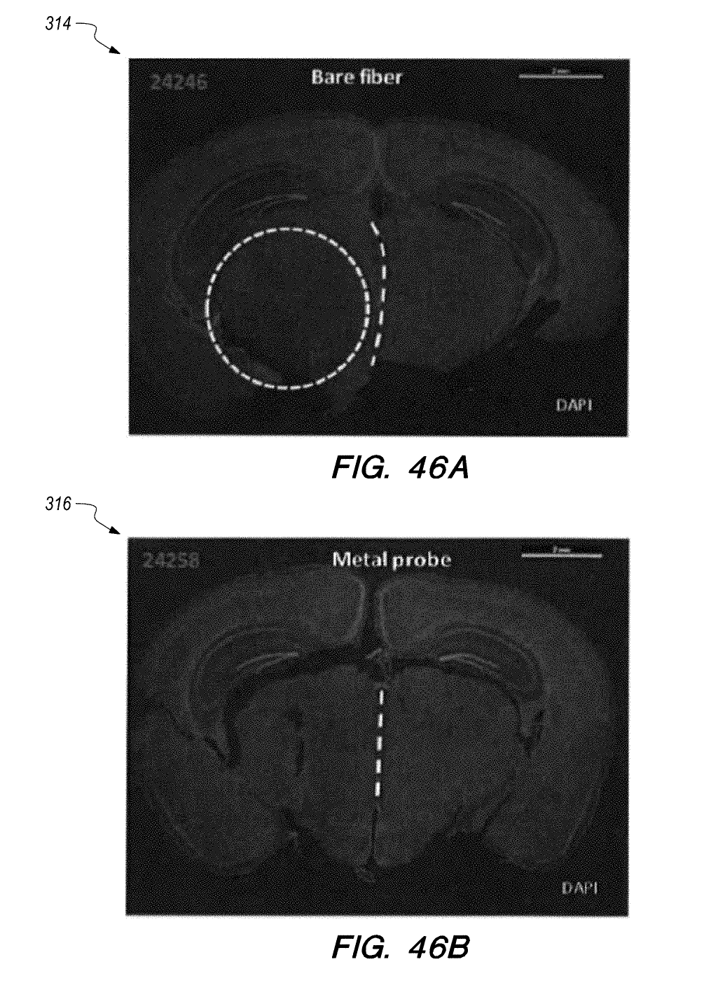

To more thoroughly investigate whether heat was the cause of the observed toxicity in the brain, we covered the optic fiber tips with metal probes. These metal probes were designed to absorb the light from the illumination treatment and transfer the energy to the brain tissue as heat. When implanted in WT mice (n=7) and tested with the same illumination parameters (see above) for 24 hours, these heat probes failed to produce the type of toxicity that was observed when the energy was delivered through a light probe, in that there was no evidence of edema or prominent loss of brain cells (Compare FIG. 46A to FIG. 46B). Image 314 of FIG. 46A is a repeated presentation of FIG. 43, placed side by side with Image 316 of FIG. 46B for comparison. Image 316 of FIG. 46B illustrates the type of neuronal damage caused solely by heat, as delivered with a metal probe. This data suggests that heating from the illumination therapy was not sufficient to drive the toxicity.

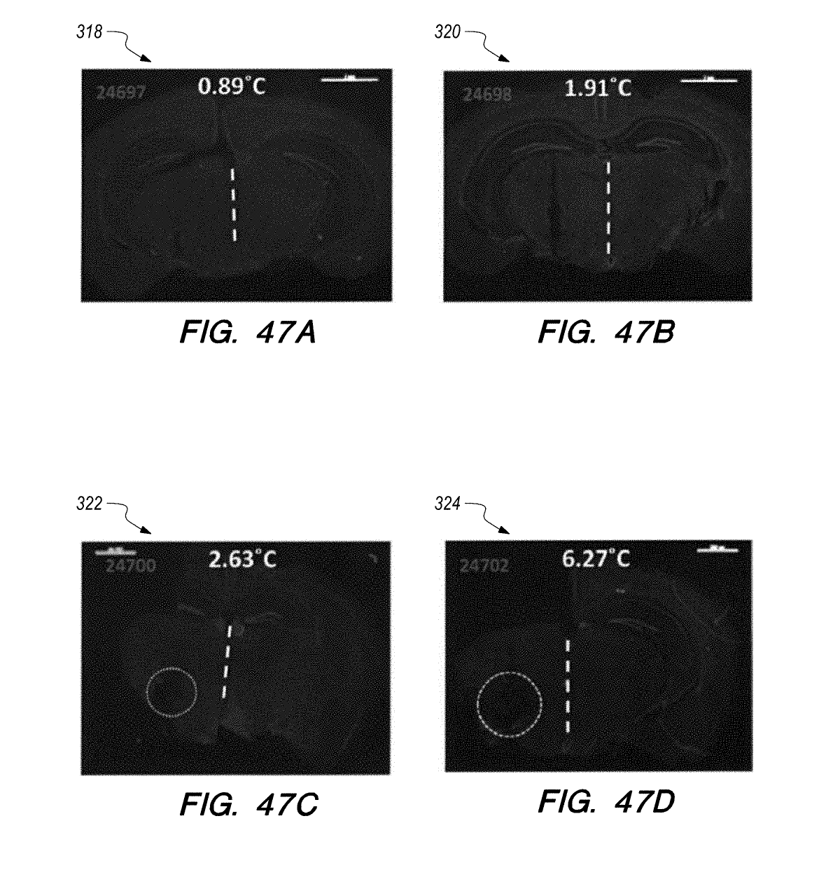

To determine the type of damage and toxicity that one might expect to observe with heating alone, we tested the same metal probes with parameters predicted to induce various temperature rises (0.9°, 1.9°, 2.6°, 4.0° and 6.3° C.) for 24 hours in the brains of WT mice. We found that even at constant heat generating a temperature rise of 6.3° C. for 24 hours, we could not replicate the type of damage observed with the original illumination treatment (see above) that generated an increase of ˜0.6° C. Although there was clear tissue disruption in the immediate vicinity of the implanted metal probe (FIGS. 47A-47D), we did not observe the extensive cell loss and edema (see FIG. 43). Image 318 of FIG. 47A illustrates the damage resulting from 0.89 degrees increase using direct delivery of heat. Image 320 of FIG. 47B shows the damage resulting from 1.91 degrees increase using direct delivery of heat. Image 322 of FIG. 47C shows the damage resulting from 2.63 degrees increase using direct delivery of heat. Image 324 of FIG. 47D shows the damage resulting from 6.27 degrees increase using direct delivery of heat. Our studies suggest that 24 hours of constant heat delivery of above ˜2.0° is required to cause disruption in the brain tissue, which corresponds to that of consensus safety standards. However, the damage generated by heat did not have the same characteristic of toxicity observed with delivery of illumination to the brain tissue, suggesting that phototoxicity might be the main mechanism of brain tissue damage.

### Example 5—Spectral Effects

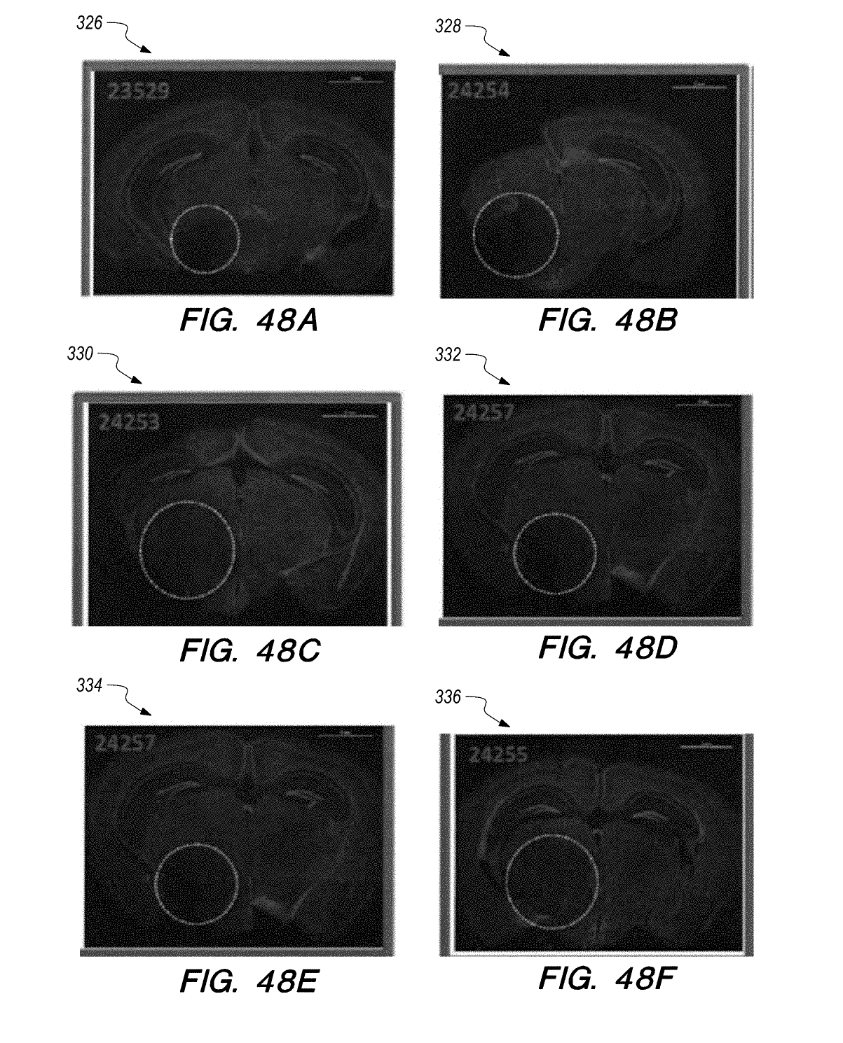

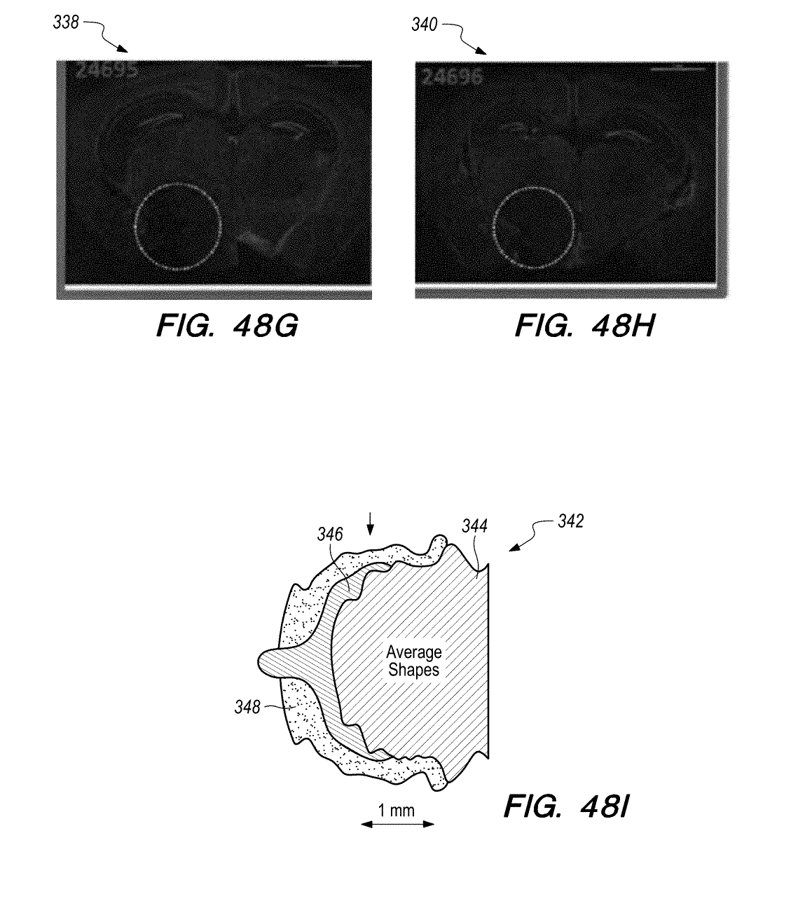

We further characterized the phototoxicity to determine whether it was generalized to the larger visual light spectrum, or specific to the red wavelength (635 nm) that we were interested in for optogenetic illumination therapy. Image 326 of FIG. 48A illustrates the damage resulting from green light administered as described in Example 5. Image 328 of FIG. 48B shows the damage resulting from green light administered as described in Example 5. Image 330 of FIG. 48C shows the damage resulting from green light administered as described in Example 5. Image 332 of FIG. 48D shows the damage resulting from green light administered as described in Example 5. Image 334 of FIG. 48E shows the damage resulting from red light administered as described in Example 5. Image 336 of FIG. 48F shows the damage resulting from red light administered as described in Example 5. Image 338 of FIG. 48G shows the damage resulting from blue light administered as described in Example 5. Image 340 of FIG. 48H shows the damage resulting from blue light administered as described in Example 5. Image 342 of FIG. 48I is a two-dimensional representation of the damage extents occurring using the three light spectra tested in Example 5 where the colors in the representation are the color of the light administered. In WT mice, we tested different visual wavelengths of blue, green, and red (473 nm n=2, FIGS. 48G, 48H; 532 nm n=4, FIGS. 48A-48D, and 635 nm n=2, FIGS. 48E, 48F) at the same parameters, as tested before, of 2 ms pulses at 250 Hz (a 50% duty cycle) and peak power of 10 mW, which was equivalent to an average power of 5 mW in the brain, resulting in a peak fluence rate of 409 mW/mm². With all wavelengths tested, we observed toxicity in the brain tissue (FIGS. 48A-48H), consistent with the idea that the damage was caused by the photons of visible light rather than heating. Furthermore, the extent or spread of the damage followed our predictive model of how far different wavelengths can penetrate in the brain (FIG. 48I). Here, we demonstrate a novel discovery that light, specifically photons in the visible light spectrum, when delivered continuously over periods 10+ hours can have toxicity effects on brain tissue.

### Example 6—Determination of Damage Threshold for Observed Phototoxicity

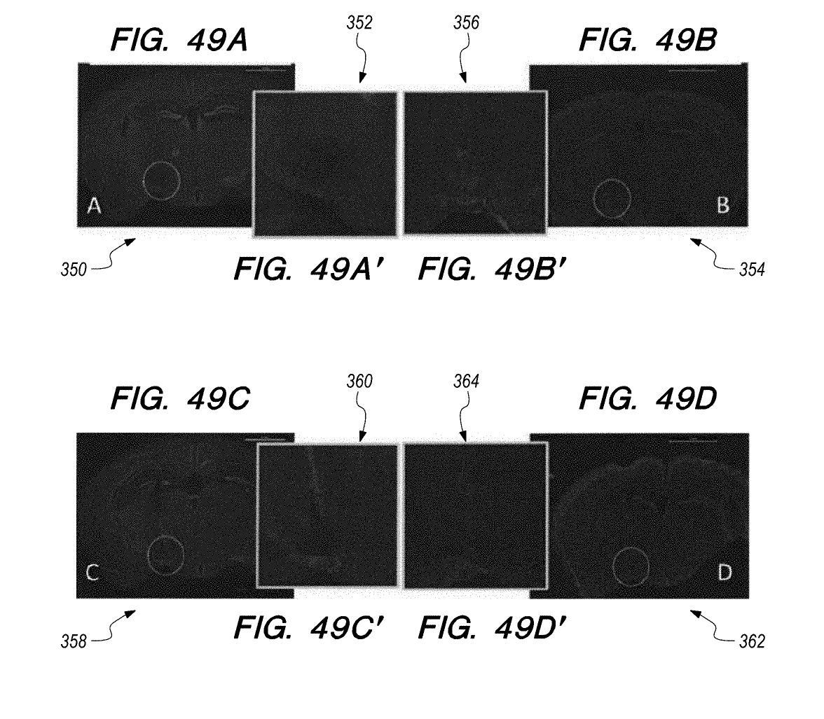

Our extensive in-vivo testing shows that there is an observable damage limit of light delivery to brain tissue. Images 350 and 352 of FIGS. 49A and 49A′, respectively, illustrate the impact upon neural tissue with 24 hours continuous light at 0.1 mW at λ=488 nm. Note that FIG. 49A′ is a magnification of the dotted area of FIG. 49A. Cellular damage and edema is evident. Images 354 and 356 of FIGS. 49B and 49B′, respectively, illustrate the impact upon neural tissue with 168 hours continuous light at 0.05 mW at 488 nm. Note that FIG. 49B′ is a magnification of the dotted area of FIG. 49B. No damage is evident. Images 358 and 360 of FIGS. 49C and 49C′, respectively, illustrate the impact upon neural tissue with 24 hours continuous light at 0.1 mW at 488 nm. Note that FIG. 49C′ is a magnification of the dotted area of FIG. 49C. Cellular damage and edema is evident. Images 362 and 364 of FIGS. 49D and 49D′, respectively, illustrate the impact upon neural tissue with 168 hours continuous light at 0.05 mW at 488 nm. Note FIGS. 49D′ is a magnification of the dotted area of FIG. 49D. No damage is evident. When testing for 24 hours, there was evidence of phototoxicity after continuous illumination with constant 520 nm and 488 nm light at levels as low as 0.1 mW, or 4 mW/mm² peak. Here, since we were testing with constant light, the peak fluence rate and average fluence rate were identical. Only when the optical power was lowered to 0.05 mW (resulting in a peak fluence rate of 2 mW/mm², and an illumination volume of 0.13 mm³ for levels ≥1 mW/mm²) was continuous illumination with constant light at 488 nm safe, in that there was no observable toxicity even after 168 hours (7 days) of continuous illumination exposure (FIGS. 49B and 49D, compare to FIGS. 49A and 49C). Thus, the safety window for delivery of light to brain tissue in-vivo, in awake behaving animals over extended periods of time may be limited. This damage threshold may be generalized to different visible wavelengths, including those wavelengths relevant to activating both existing excitatory and inhibitory opsins, such as those within the range of 400 nm to 650 nm. Thus, the prospect of translating optogenetics into viable direct therapies for patients with neurological disorders may face a challenge of low tolerance to phototoxicity in brain tissue. However, to support our efforts to translate optogenetics into a clinical setting, we have discovered that we can extend the safety window for phototoxicity by using different pulse patterns, and we also identified opsins that are functional well below the damage threshold.

### Example 7—Functional and Safe Inhibitory Opsin: SwiChR++

We have confirmed a solution for the damage limit of low tolerance to phototoxicity of brain tissue by demonstrating the efficacy of an ultra-sensitive inhibitory opsin, SwiChR++(Berndt et al. 2016 Berndt, A. et al., 2016. Structural foundations of optogenetics: Determinants of channelrhodopsin ion selectivity. Proceedings of the National Academy of Sciences, 113(4), pp. 822-829, see also WO2015148974; each of which is incorporated by reference herein in its entirety), that operates well below the damage threshold. SwiChR++ is an enhanced chloride channel, developed by principled structure-guided approach to engineering channelrhodopsin for chloride selectivity. This opsin may be activated with blue light, inhibits neuronal activity when open, and remains open for prolonged periods because of its extended time constant for closing, thus conferring it extreme sensitivity to light. We first determined whether SwiChR++ was functional as an inhibitory opsin, and whether it could provide therapeutic relief in the 60HDA lesioned rat PD model.

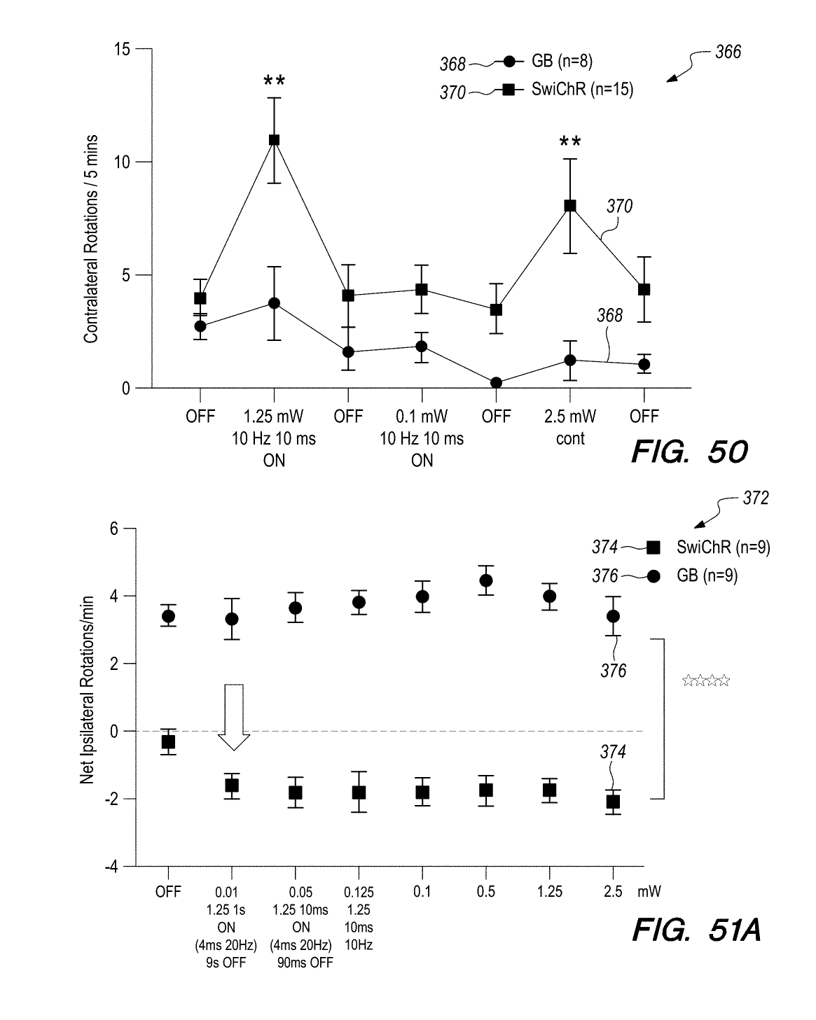

For this study, male rats, with adequate 60HDA lesions (confirmed by apomorphine screening) received unilateral STN injections of vector for expression of SwiChR++, ipsilateral to the side of the lesion. Injections consisted of 300 nl of vehicle control (gradient buffer, GB, n=8) or 300 nl of AAV1-hsyn-SwiChR++-P2A (n=15) at the concentration of 1.0×10¹³ vg/mL (total amount, 3.0×10⁹ vg). There was a light-mediated therapeutic effect in the lesioned rats that expressed SwiChR++, in that the rats showed a robust increase in rotations contralateral to the side of the lesion, as expected for a therapeutic effect (FIG. 50). Plot 366 of FIG. 50 shows the results of the light mediated therapeutic effect using 60HDA rats with administration regimes of 1.25 mW/10 Hz/10 ms; 0.1 mW/10 Hz/10 ms; and 2.5 mW continuous light. The effect was significant for constant illumination of 2.5 mW (resulting in a peak fluence rate of 102 mW/mm², and an illumination volume of 0.57 mm³ for levels ≥1 mW/mm²), as well as for an average power of 0.125 mW (1.25 mW peak with 10 ms pulses at 10 Hz (a duty cycle of 10%, and resulting in a peak fluence rate of 50 mW/mm², and an illumination volume of 0.26=0 for levels ≥1 mW/mm²). These results suggest that SwiChR++ is indeed a functional inhibitory opsin that may provide therapeutic relief in the PD rodent model and translate to use in humans. Data 368 and 370 represent experimental and control groups, respectively.

### Example 8—SwiChR++ Function in 60HDA Lesioned Mice

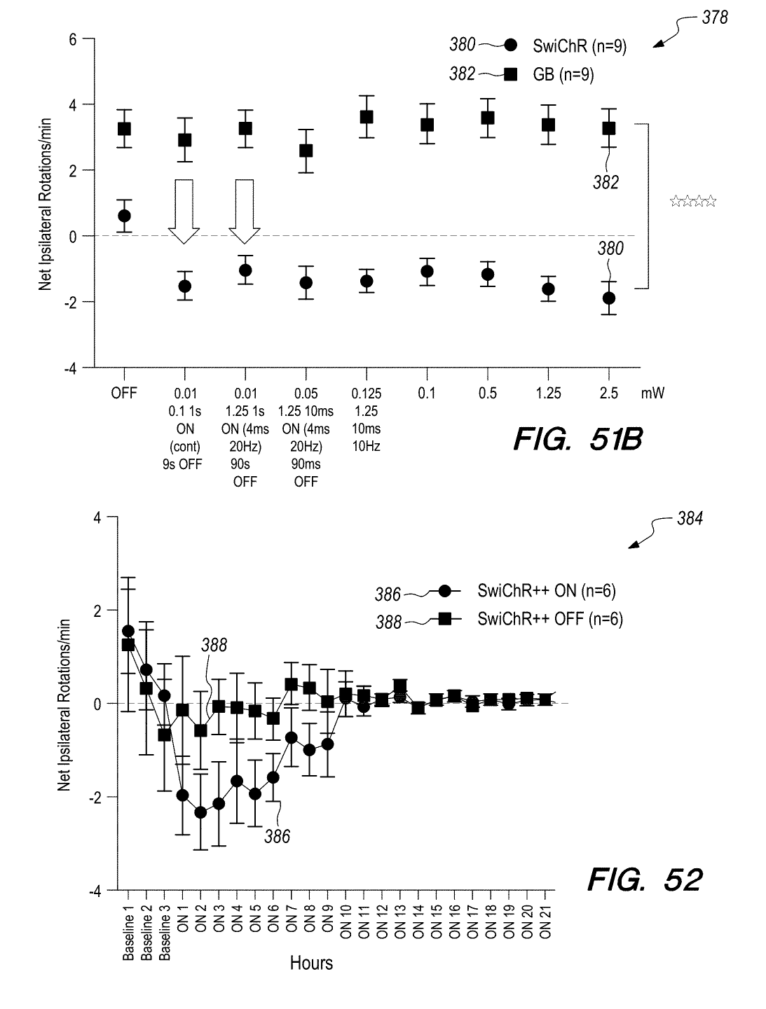

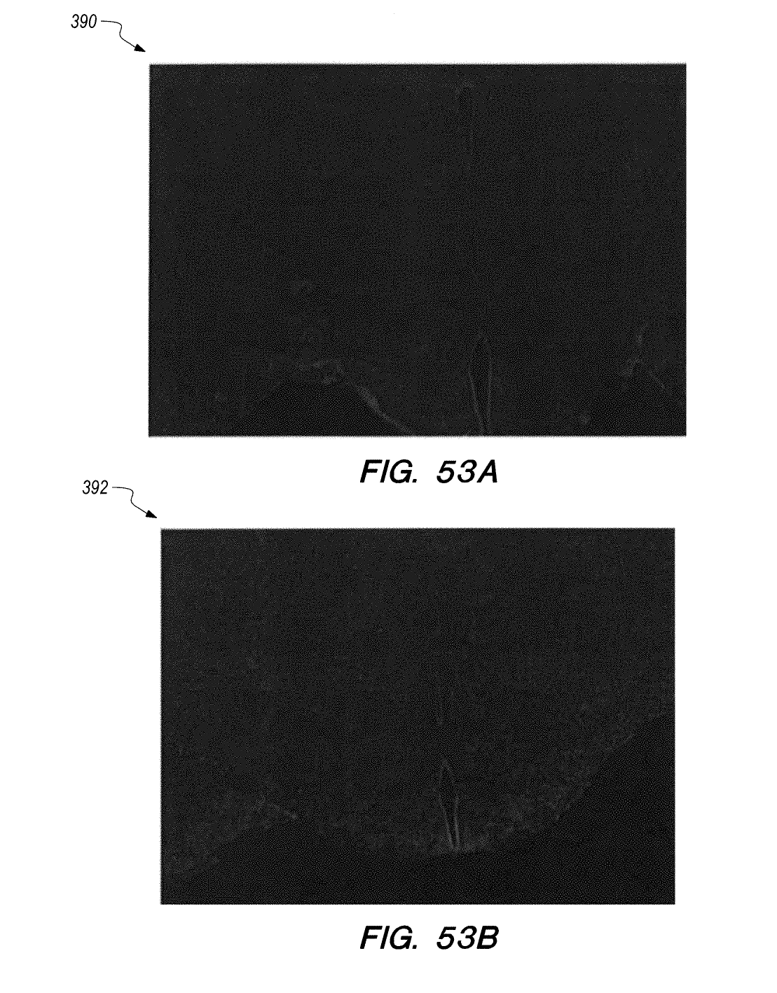

Then, we sought to confirm the efficacy of SwiChR++ in a second species, and tested whether SwiChR++ was functional with light level below the damage limit. As describe before, male mice, with adequate unilateral 60HDA lesions confirmed by exceeding a threshold of rotations in response to the dopamine agonist apomorphine, received unilateral STN injections of SwiChR++, ipsilateral to the side of the lesion. Injections consisted of 500 nl of vehicle control (gradient buffer, GB, n=9) or 500 nl of the inhibitory opsin (AAV1-hsyn-SwiChr++-p2A) (n=9) at the concentration of 1.04×10¹³ vg/mL (total titer amount of 5.20×10⁹ vg). After injection of vector by stereotaxic surgery, animals were also implanted with an optic fiber for light delivery to the STN that went into the brain with the fiber tip positioned approximately 0.25 mm above the STN. After four weeks of expression, we found that SwiChR++ produced a robust therapeutic effect, as measured by the rotation assay, with light levels that were well below the threshold for phototoxicity (FIGS. 51A and 51B, red arrows). Plot 372 of FIG. 51A documents, at 4.5 weeks expression of the opsin, the decrease in net ispsilaterial rotations per minute with various light administration regimes of 0.01 mW (1.25 mW is ON, 4 ms 20 Hz, 9 s OFF); 0.05 mW (1.25 mW 10 ms ON (4 ms, 20 Hz, 90 ms OFF); 0.125 mW (1.25 m@ 10 ms ON, 10 Hz); 0.1 mW; 0.5 mW; 1.25 mW; and 2.5 mW. Data 374 and 376 represent experimental and control groups, respectively. Plot 378 of FIG. 51B documents, at 5 weeks expression of the opsin, the decrease in net ispsilaterial rotations per minute with various light administration regimes of 0.01 mW (1.25 mW is ON, 4 ms 20 Hz, 9 s OFF); 0.05 mW (1.25 mW 10 ms ON (4 ms, 20 Hz, 90 ms OFF); 0.125 mW (1.25 m@ 10 ms ON, 10 Hz); 0.1 mW; 0.5 mW; 1.25 mW; and 2.5 mW. The magnitude of the therapeutic response was comparable across different patterns of light and different average light intensities, suggesting that SwiChR++ is indeed an ultrasensitive channel for which the effect saturates at very low light level. The therapeutic effect was reproducible week after week. Furthermore, the efficacy of SwiChR++ was well maintained during continuous testing for 24 hours (FIG. 52). Data 380 and 382 represent experimental and control groups, respectively. Plot 384 of FIG. 52 documents the therapeutic effect of SwiChR++ activation over 21 hours of light ON. We found a significant difference when comparing SwiChR++ animals that received the illumination treatment (ON) vs. animals without light treatment (OFF). This effect was evident during the active (dark) cycle of the mice, and there was no indication of disruption of sleep during the inactive (light) cycle of the mice. These experiments were conducted by taking advantage of the long time constant of the SwiChR++ channel to close following activation: we were able to reduce the total average amount of light delivered to the average light delivery of 0.002 mW over the period of light ‘ON’ testing (10 ms pulses at 10 Hz for 1 minute every 5 minutes (an overall duty cycle of 2%) with a peak power of 0.1 mW at 488 nm), resulting in a peak fluence rate of 4 mW/mm², and an illumination volume of 0.19 mm³ for levels ≥0.1 mW/mm². This average exposure was 25Xs less than that of cw exposure (0.05 mW, 2 mW/mm2 peak,) and 50Xs less than the level of pulsed light (average 0.1 mW, 1.25 mW peak, 53 mW/mm2 peak, 10 ms at 10 Hz) we found to be safe and not cause toxicity in brain tissue when tested continuously for 168 hours (7 days) in WT mice (FIGS. 53A and 53B). Data 386 and 388 represent experimental and control groups, respectively. Image 390 of FIG. 53A illustrates a lack of cellular damage with 168 hours of continuous light at an average administration of 0.05 mW of light (488 nm, constant ON). Image 392 of FIG. 53B illustrates lack of cellular damage with 168 hours of light at an average administration of 0.1 mW (488 nm, 10 ms, 10 Hz). Thus, we have demonstrated efficacy with an opsin that functions well below our newly discovered damage limits of brain tissue phototoxicity.

Recalling the descriptions of damage limits and exposure durations and the means of calculating the fluence rate in tissue, we can see from the above examples that for continuous exposures (i.e. durations >2 hrs over 24 hrs), an average fluence rate of ≤2 mW/mm² and peak fluence rate of <50 mw/mm² when using a duty cycle ≤10% may define the safety range for the use of visible light within most areas of the brain, along with the use of pulse durations of between 0.1 ms and 1 s, and pulse intervals of between 2.5 ms and 10 s, (or frequencies of between 0.1 Hz and 400 Hz). Duty cycles may be adjusted to provide limits by average power to peak power constraints, as it may appear that the pulse interval provides time to maintain cellular health. For a train of pulses the limit set by the pulse durations above may also apply to the pulse burst time. For treatment durations <2 hrs within a 24 hour period, the average fluence rate may approach the peak fluence rate. Modeling calculates that 2 mW/mm² corresponds to <0.1 mW out of a 200 um diameter fiber, and 50 mw/mm² to <1.25 mW out of a 200 um diameter fiber in grey matter with a 4% volumetric blood concentration.

The opsin protein may be selected from the group consisting of: a depolarizing opsin, a hyperpolarizing opsin, a stimulatory opsin, an inhibitory opsin, a chimeric opsin, and a step-function opsin. The opsin protein may be selected from the group consisting of: NpHR, eNpHR 1.0, eNpHR 2.0, eNpHR 3.0, SwiChR, SwiChR 2.0, SwiChR 3.0, Mac, Mac 3.0, Arch, ArchT, Arch 3.0, ArchT 3.0, iChR, ChR2, C1V1-T, C1V1-TT, Chronos, Chrimson, ChrimsonR, CatCh, VChR1-SFO, ChR2-SFO, ChR2-SSFO, ChEF, ChIEF, Jaws, ChloC, Slow ChloC, iC1C2, iC1C2 2.0, and iC1C2 3.0. For example, an “inhibitory” channel (such as those referred to as “iChR” or “SwiChR”) may be utilized to open and permit large amounts of Cl− ions to pass, thereby hyperpolarizing the neuron more effectively and thus inhibiting the cell with efficiency and sensitivity. These opsins have action spectra similar to that of ChR and ChR2, with a peak spectral response at about 460 nm.

In some embodiments, the light-responsive protein can comprise an amino acid sequence at least about 90%, 91%, 92%, 93%, 94%, 95%, 96%, 97%, 98%, 99%, or 100% identical to the sequence shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:49. In an embodiment, the light-responsive protein can comprise an amino acid sequence at least about 90%, 91%, 92%, 93%, 94%, 95%, 96%, 97%, 98%, 99%, or 100% identical to a polypeptide encoded by SEQ ID NO:50.

An “individual” can be a mammal, including a human. Mammals include, but are not limited to, farm animals, sport animals, pets, primates, mice and rats. Individuals also include companion animals including, but not limited to, dogs and cats. In one aspect, an individual is a human. In another aspect, an individual is a non-human animal.

As used herein, “depolarization-induced synaptic depletion” occurs when continuous depolarization of a neural cell plasma membrane prevents the neural cell from sustaining high frequency action on efferent targets due to depletion of terminal vesicular stores of neurotransmitters.

Amino acid substitutions in a native protein sequence may be “conservative” or “non-conservative” and such substituted amino acid residues may or may not be one encoded by the genetic code. A “conservative amino acid substitution” is one in which the amino acid residue is replaced with an amino acid residue having a chemically similar side chain (i.e., replacing an amino acid possessing a basic side chain with another amino acid with a basic side chain). A “non-conservative amino acid substitution” is one in which the amino acid residue is replaced with an amino acid residue having a chemically different side chain (i.e., replacing an amino acid having a basic side chain with an amino acid having an aromatic side chain).

The standard twenty amino acid “alphabet” is divided into chemical families based on chemical properties of their side chains. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and side chains having aromatic groups (e.g., tyrosine, phenylalanine, tryptophan, histidine).

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “treatment” or “treating” is an approach for obtaining beneficial or desired results including clinical results. For purposes of this invention, beneficial or desired clinical results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals.

### Light-Responsive Opsin Proteins

Provided herein are optogenetic-based methods for selectively hyperpolarizing or depolarizing neurons.

Optogenetics refers to the combination of genetic and optical methods used to control specific events in targeted cells of living tissue, even within freely moving mammals and other animals, with the temporal precision (millisecond-timescale) needed to keep pace with functioning intact biological systems. Optogenetics requires the introduction of fast light-responsive channel or pump proteins to the plasma membranes of target neuronal cells that allow temporally precise manipulation of neuronal membrane potential while maintaining cell-type resolution through the use of specific targeting mechanisms. Any microbial opsin that can be used to promote neural cell membrane hyperpolarization or depolarization in response to light may be used. For example, the Halorhodopsin family of light-responsive chloride pumps (e.g., NpHR, NpHR2.0, NpHR3.0, NpHR3.1) and the GtR3 proton pump can be used to promote neural cell membrane hyperpolarization in response to light. As another example, eARCH (a proton pump) or ArchT can be used to promote neural cell membrane hyperpolarization in response to light. Additionally, members of the Channelrhodopsin family of light-responsive cation channel proteins (e.g., ChR2, SFOs, SSFOs, C1V1 s) can be used to promote neural cell membrane depolarization or depolarization-induced synaptic depletion in response to a light stimulus.

### Enhanced Intracellular Transport Amino Acid Motifs

The present disclosure provides for the modification of light-responsive opsin proteins expressed in a cell by the addition of one or more amino acid sequence motifs which enhance transport to the plasma membranes of mammalian cells. Light-responsive opsin proteins having components derived from evolutionarily simpler organisms may not be expressed or tolerated by mammalian cells or may exhibit impaired subcellular localization when expressed at high levels in mammalian cells. Consequently, in some embodiments, the light-responsive opsin proteins expressed in a cell can be fused to one or more amino acid sequence motifs selected from the group consisting of a signal peptide, an endoplasmic reticulum (ER) export signal, a membrane trafficking signal, and/or an N-terminal golgi export signal. The one or more amino acid sequence motifs which enhance light-responsive protein transport to the plasma membranes of mammalian cells can be fused to the N-terminus, the C-terminus, or to both the N- and C-terminal ends of the light-responsive protein. Optionally, the light-responsive protein and the one or more amino acid sequence motifs may be separated by a linker. In some embodiments, the light-responsive protein can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:37).

Trafficking sequences that are suitable for use can comprise an amino acid sequence having 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:37)).

A trafficking sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

Signal sequences that are suitable for use can comprise an amino acid sequence having 90%, 91%, 92%, 93%, 94%, 95%, 96%, 97%, 98%, 99%, or 100%, amino acid sequence identity to an amino acid sequence such as one of the following:

1) the signal peptide of hChR2 (e.g., MDYGGALSAVGRELLFVTNPVVVNGS (SEQ ID NO:38))
2) the .beta.2 subunit signal peptide of the neuronal nicotinic acetylcholine receptor (e.g., MAGHSNSMALFSFSLLWLCSGVLGTEF (SEQ ID NO:39));
3) a nicotinic acetylcholine receptor signal sequence (e.g., MGLRALMLWLLAAAGLVRESLQG (SEQ ID NO:40)); and
4) a nicotinic acetylcholine receptor signal sequence (e.g., MRGTPLLLVVSLFSLLQD (SEQ ID NO:41)).

A signal sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

Endoplasmic reticulum (ER) export sequences that are suitable for use in a modified opsin of the present disclosure include, e.g., VXXSL (where X is any amino acid) [SEQ ID NO:42] (e.g., VKESL (SEQ ID NO:43); VLGSL (SEQ ID NO:44); etc.); NANSFCYENEVALTSK (SEQ ID NO:45); FXYENE (SEQ ID NO:46) (where X is any amino acid), e.g., FCYENEV (SEQ ID NO:47); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

Additional protein motifs which can enhance light-responsive protein transport to the plasma membrane of a cell are described in U.S. patent application Ser. No. 12/041,628, which is incorporated herein by reference in its entirety. In some embodiments, the signal peptide sequence in the protein can be deleted or substituted with a signal peptide sequence from a different protein.

### Light-Responsive Chloride Pumps

In some aspects of the methods provided herein, one or more members of the Halorhodopsin family of light-responsive chloride pumps are expressed on the plasma membranes of neural cells.

In some aspects, said one or more light-responsive chloride pump proteins expressed on the plasma membranes of the nerve cells described above can be derived from Natronomonas pharaonic. In some embodiments, the light-responsive chloride pump proteins can be responsive to amber light as well as red light and can mediate a hyperpolarizing current in the nerve cell when the light-responsive chloride pump proteins are illuminated with amber or red light. The wavelength of light which can activate the light-responsive chloride pumps can be between about 580 and 630 nm. In some embodiments, the light can be at a wavelength of about 589 nm or the light can have a wavelength greater than about 630 nm (e.g. less than about 740 nm). In another embodiment, the light has a wavelength of around 630 nm. In some embodiments, the light-responsive chloride pump protein can hyperpolarize a neural membrane for at least about 90 minutes when exposed to a continuous pulse of light. In some embodiments, the light-responsive chloride pump protein can comprise an amino acid sequence at least about 90%, 91%, 92%, 93%, 94%, 95%, 96%, 97%, 98%, 99%, or 100% identical to the sequence shown in SEQ ID NO:32. Additionally, the light-responsive chloride pump protein can comprise substitutions, deletions, and/or insertions introduced into a native amino acid sequence to increase or decrease sensitivity to light, increase or decrease sensitivity to particular wavelengths of light, and/or increase or decrease the ability of the light-responsive protein to regulate the polarization state of the plasma membrane of the cell. In some embodiments, the light-responsive chloride pump protein contains one or more conservative amino acid substitutions. In some embodiments, the light-responsive protein contains one or more non-conservative amino acid substitutions.

The light-responsive protein comprising substitutions, deletions, and/or insertions introduced into the native amino acid sequence suitably retains the ability to hyperpolarize the plasma membrane of a neuronal cell in response to light.

Additionally, in other aspects, the light-responsive chloride pump protein can comprise a core amino acid sequence at least about 90%, 91%, 92%, 93%, 94%, 95%, 96%, 97%, 98%, 99%, or 100% identical to the sequence shown in SEQ ID NO: 32 and an endoplasmic reticulum (ER) export signal. This ER export signal can be fused to the C-terminus of the core amino acid sequence or can be fused to the N-terminus of the core amino acid sequence. In some embodiments, the ER export signal is linked to the core amino acid sequence by a linker. The linker can comprise any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments, the ER export signal can comprise the amino acid sequence FXYENE (SEQ ID NO:46), where X can be any amino acid. In another embodiment, the ER export signal can comprise the amino acid sequence VXXSL, where X can be any amino acid [SEQ ID NO:42]. In some embodiments, the ER export signal can comprise the amino acid sequence FCYENEV (SEQ ID NO:47).

Endoplasmic reticulum (ER) export sequences that are suitable for use in a modified opsin of the present disclosure include, e.g., VXXSL (where X is any amino acid) [SEQ ID NO:42] (e.g., VKESL (SEQ ID NO:43); VLGSL (SEQ ID NO:44); etc.); NANSFCYENEVALTSK (SEQ ID NO:45); FXYENE (where X is any amino acid) (SEQ ID NO:46), e.g., FCYENEV (SEQ ID NO:47); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

In other aspects, the light-responsive chloride pump proteins provided herein can comprise a light-responsive protein expressed on the cell membrane, wherein the protein comprises a core amino acid sequence at least about 90%, 91%, 92%, 93%, 94%, 95%, 96%, 97%, 98%, 99%, or 100% identical to the sequence shown in SEQ ID NO: 32 and a trafficking signal (e.g., which can enhance transport of the light-responsive chloride pump protein to the plasma membrane). The trafficking signal may be fused to the C-terminus of the core amino acid sequence or may be fused to the N-terminus of the core amino acid sequence. In some embodiments, the trafficking signal can be linked to the core amino acid sequence by a linker which can comprise any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:37).

In some aspects, the light-responsive chloride pump protein can comprise a core amino acid sequence at least about 90%, 91%, 92%, 93%, 94%, 95%, 96%, 97%, 98%, 99%, or 100% identical to the sequence shown in SEQ ID NO: 32 and at least one (such as one, two, three, or more) amino acid sequence motifs which enhance transport to the plasma membranes of mammalian cells selected from the group consisting of an ER export signal, a signal peptide, and a membrane trafficking signal. In some embodiments, the light-responsive chloride pump protein comprises an N-terminal signal peptide, a C-terminal ER Export signal, and a C-terminal trafficking signal. In some embodiments, the C-terminal ER Export signal and the C-terminal trafficking signal can be linked by a linker. The linker can comprise any of about 5, 10, 20, 30, 40, 50, 75, 100, 125, 150, 175, 200, 225, 250, 275, 300, 400, or 500 amino acids in length. The linker can also further comprise a fluorescent protein, for example, but not limited to, a yellow fluorescent protein, a red fluorescent protein, a green fluorescent protein, or a cyan fluorescent protein. In some embodiments the ER Export signal can be more C-terminally located than the trafficking signal. In other embodiments the trafficking signal is more C-terminally located than the ER Export signal. In some embodiments, the signal peptide comprises the amino acid sequence MTETLPPVTESAVALQAE (SEQ ID NO:48). In another embodiment, the light-responsive chloride pump protein comprises an amino acid sequence at least 95% identical to SEQ ID NO:33.

Moreover, in other aspects, the light-responsive chloride pump proteins can comprise a core amino acid sequence at least about 90%, 91%, 92%, 93%, 94%, 95%, 96%, 97%, 98%, 99%, or 100% identical to the sequence shown in SEQ ID NO: 32, wherein the N-terminal signal peptide of SEQ ID NO:32 is deleted or substituted. In some embodiments, other signal peptides (such as signal peptides from other opsins) can be used. The light-responsive protein can further comprise an ER transport signal and/or a membrane trafficking signal described herein. In some embodiments, the light-responsive chloride pump protein comprises an amino acid sequence at least 95% identical to SEQ ID NO:34.

In some embodiments, the light-responsive opsin protein is a NpHR opsin protein comprising an amino acid sequence at least 950, at least 960, at least 970, at least 980, at least 99% or 100% identical to the sequence shown in SEQ ID NO:32. In some embodiments, the NpHR opsin protein further comprises an endoplasmic reticulum (ER) export signal and/or a membrane trafficking signal. For example, the NpHR opsin protein comprises an amino acid sequence at least 95% identical to the sequence shown in SEQ ID NO:32 and an endoplasmic reticulum (ER) export signal. In some embodiments, the amino acid sequence at least 95% identical to the sequence shown in SEQ ID NO:32 is linked to the ER export signal through a linker. In some embodiments, the ER export signal comprises the amino acid sequence FXYENE (SEQ ID NO:46), where X can be any amino acid. In another embodiment, the ER export signal comprises the amino acid sequence VXXSL, where X can be any amino acid [SEQ ID NO:42]. In some embodiments, the ER export signal comprises the amino acid sequence FCYENEV (SEQ ID NO:47). In some embodiments, the NpHR opsin protein comprises an amino acid sequence at least 95% identical to the sequence shown in SEQ ID NO:32, an ER export signal, and a membrane trafficking signal. In other embodiments, the NpHR opsin protein comprises, from the N-terminus to the C-terminus, the amino acid sequence at least 95% identical to the sequence shown in SEQ ID NO:32, the ER export signal, and the membrane trafficking signal. In other embodiments, the NpHR opsin protein comprises, from the N-terminus to the C-terminus, the amino acid sequence at least 95% identical to the sequence shown in SEQ ID NO:32, the membrane trafficking signal, and the ER export signal. In some embodiments, the membrane trafficking signal is derived from the amino acid sequence of the human inward rectifier potassium channel Kir2.1. In some embodiments, the membrane trafficking signal comprises the amino acid sequence K S R I T S E G E Y I P L D Q I D I N V (SEQ ID NO:37). In some embodiments, the membrane trafficking signal is linked to the amino acid sequence at least 95% identical to the sequence shown in SEQ ID NO:32 by a linker. In some embodiments, the membrane trafficking signal is linked to the ER export signal through a linker. The linker may comprise any of 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments, the light-responsive opsin protein further comprises an N-terminal signal peptide. In some embodiments, the light-responsive opsin protein comprises the amino acid sequence of SEQ ID NO:33. In some embodiments, the light-responsive opsin protein comprises the amino acid sequence of SEQ ID NO:34.

Also provided herein are polynucleotides encoding any of the light-responsive chloride ion pump proteins described herein, such as a light-responsive protein comprising a core amino acid sequence at least about 90%, 91%, 92%, 93%, 94%, 95%, 96%, 97%, 98%, 99%, or 100% identical to the sequence shown in SEQ ID NO:32, an ER export signal, and a membrane trafficking signal. In another embodiment, the polynucleotides comprise a sequence which encodes an amino acid at least 95% identical to SEQ ID NO:33 and SEQ ID NO:34. The polynucleotides may be in an expression vector (such as, but not limited to, a viral vector described herein). The polynucleotides may be used for expression of the light-responsive chloride ion pump proteins.

Further disclosure related to light-responsive chloride pump proteins can be found in U.S. Patent Application Publication Nos: 2009/0093403 and 2010/0145418 as well as in International Patent Application No: PCT/US2011/028893, the disclosures of each of which are hereby incorporated by reference in their entireties.

### Light-Responsive Proton Pumps

In some aspects of the methods provided herein, one or more light-responsive proton pumps are expressed on the plasma membranes of the neural cells.

In some embodiments, the light-responsive proton pump protein can be responsive to blue light and can be derived from Guillardia theta, wherein the proton pump protein can be capable of mediating a hyperpolarizing current in the cell when the cell is illuminated with blue light. The light can have a wavelength between about 450 and about 495 nm or can have a wavelength of about 490 nm. In another embodiment, the light-responsive proton pump protein can comprise an amino acid sequence at least about 90%, 91%, 92%, 93%, 94%, 95%, 96%, 97%, 98%, 99%, or 1000 identical to the sequence shown in SEQ ID NO:31. The light-responsive proton pump protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-responsive proton pump protein to regulate the polarization state of the plasma membrane of the cell. Additionally, the light-responsive proton pump protein can contain one or more conservative amino acid substitutions and/or one or more non-conservative amino acid substitutions. The light-responsive proton pump protein comprising substitutions, deletions, and/or insertions introduced into the native amino acid sequence suitably retains the ability to hyperpolarize the plasma membrane of a neuronal cell in response to light.

In other aspects of the methods disclosed herein, the light-responsive proton pump protein can comprise a core amino acid sequence at least about 90%, 91%, 92%, 93%, 94%, 95%, 96%, 97%, 98%, 99%, or 100% identical to the sequence shown in SEQ ID NO:31 and at least one (such as one, two, three, or more) amino acid sequence motifs which enhance transport to the plasma membranes of mammalian cells selected from the group consisting of a signal peptide, an ER export signal, and a membrane trafficking signal. In some embodiments, the light-responsive proton pump protein comprises an N-terminal signal peptide and a C-terminal ER export signal. In some embodiments, the light-responsive proton pump protein comprises an N-terminal signal peptide and a C-terminal trafficking signal. In some embodiments, the light-responsive proton pump protein comprises an N-terminal signal peptide, a C-terminal ER Export signal, and a C-terminal trafficking signal. In some embodiments, the light-responsive proton pump protein comprises a C-terminal ER Export signal and a C-terminal trafficking signal. In some embodiments, the C-terminal ER Export signal and the C-terminal trafficking signal are linked by a linker. The linker can comprise any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER Export signal is more C-terminally located than the trafficking signal. In some embodiments the trafficking signal is more C-terminally located than the ER Export signal.

Also provided herein are isolated polynucleotides encoding any of the light-responsive proton pump proteins described herein, such as a light-responsive proton pump protein comprising a core amino acid sequence at least about 90%, 91%, 92%, 93%, 94%, 95%, 96%, 97%, 98%, 99%, or 100% identical to the sequence shown in SEQ ID NO:31. Also provided herein are expression vectors (such as a viral vector described herein) comprising a polynucleotide encoding the proteins described herein, such as a light-responsive proton pump protein comprising a core amino acid sequence at least about 90%, 91%, 92%, 93%, 94%, 95%, 96%, 97%, 98%, 99%, or 100% identical to the sequence shown in SEQ ID NO:31. The polynucleotides may be used for expression of the light-responsive protein in neural cells.

Further disclosure related to light-responsive proton pump proteins can be found in International Patent Application No. PCT/US2011/028893, the disclosure of which is hereby incorporated by reference in its entirety.

In some embodiments, the light-responsive proton pump protein can be responsive to green or yellow light and can be derived from Halorubrum sodomense or Halorubrum sp. TP009, wherein the proton pump protein can be capable of mediating a hyperpolarizing current in the cell when the cell is illuminated with green or yellow light. The light can have a wavelength between about 560 and about 570 nm or can have a wavelength of about 566 nm. In another embodiment, the light-responsive proton pump protein can comprise an amino acid sequence at least about 90%, 91%, 92%, 93%, 94%, 95%, 96%, 97%, 98%, 99%, or 100% identical to the sequence shown in SEQ ID NO:25 or SEQ ID NO:26. The light-responsive proton pump protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-responsive proton pump protein to regulate the polarization state of the plasma membrane of the cell. Additionally, the light-responsive proton pump protein can contain one or more conservative amino acid substitutions and/or one or more non-conservative amino acid substitutions. The light-responsive proton pump protein comprising substitutions, deletions, and/or insertions introduced into the native amino acid sequence suitably retains the ability to hyperpolarize the plasma membrane of a neuronal cell in response to light.

In other aspects of the methods disclosed herein, the light-responsive proton pump protein can comprise a core amino acid sequence at least about 900, 910, 920, 930, 940, 950, 960, 970, 980, 990, or 100% identical to the sequence shown in SEQ ID NO:25 or SEQ ID NO:26 and at least one (such as one, two, three, or more) amino acid sequence motifs which enhance transport to the plasma membranes of mammalian cells selected from the group consisting of a signal peptide, an ER export signal, and a membrane trafficking signal. In some embodiments, the light-responsive proton pump protein comprises an N-terminal signal peptide and a C-terminal ER export signal. In some embodiments, the light-responsive proton pump protein comprises an N-terminal signal peptide and a C-terminal trafficking signal. In some embodiments, the light-responsive proton pump protein comprises an N-terminal signal peptide, a C-terminal ER Export signal, and a C-terminal trafficking signal. In some embodiments, the light-responsive proton pump protein comprises a C-terminal ER Export signal and a C-terminal trafficking signal. In some embodiments, the C-terminal ER Export signal and the C-terminal trafficking signal are linked by a linker. The linker can comprise any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER Export signal is more C-terminally located than the trafficking signal. In some embodiments the trafficking signal is more C-terminally located than the ER Export signal.

Also provided herein are isolated polynucleotides encoding any of the light-responsive proton pump proteins described herein, such as a light-responsive proton pump protein comprising a core amino acid sequence at least about 90%, 91%, 92%, 93%, 94%, 95%, 96%, 97%, 98%, 99%, or 100% identical to the sequence shown in SEQ ID NO:25 or SEQ ID NO:26. Also provided herein are expression vectors (such as a viral vector described herein) comprising a polynucleotide encoding the proteins described herein, such as a light-responsive proton pump protein comprising a core amino acid sequence at least about 90%, 91%, 92%, 93%, 94%, 95%, 96%, 97%, 98%, 99%, or 100% identical to the sequence shown in SEQ ID NO:25 or SEQ ID NO:26. The polynucleotides may be used for expression of the light-responsive protein in neural cells.

### Light-Responsive Cation Channel Proteins

In some aspects of the methods provided herein, one or more light-responsive cation channels can be expressed on the plasma membranes of the neural cells.

In some aspects, the light-responsive cation channel protein can be derived from Chlamydomonas reinhardtii, wherein the cation channel protein can be capable of mediating a depolarizing current in the cell when the cell is illuminated with light. In another embodiment, the light-responsive cation channel protein can comprise an amino acid sequence at least about 90%, 91%, 92%, 93%, 94%, 95%, 96%, 97%, 98%, 99%, or 100% identical to the sequence shown in SEQ ID NO:1. The light used to activate the light-responsive cation channel protein derived from Chlamydomonas reinhardtii can have a wavelength between about 460 and about 495 nm or can have a wavelength of about 480 nm. Additionally, the light can have an intensity of at least about 100 Hz. In some embodiments, activation of the light-responsive cation channel derived from Chlamydomonas reinhardtii with light having an intensity of 100 Hz can cause depolarization-induced synaptic depletion of the neurons expressing the light-responsive cation channel. The light-responsive cation channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-responsive cation channel protein to regulate the polarization state of the plasma membrane of the cell. Additionally, the light-responsive cation channel protein can contain one or more conservative amino acid substitutions and/or one or more non-conservative amino acid substitutions. The light-responsive proton pump protein comprising substitutions, deletions, and/or insertions introduced into the native amino acid sequence suitably retains the ability to depolarize the plasma membrane of a neuronal cell in response to light.

In some embodiments, the light-responsive cation channel comprises a T159C substitution of the amino acid sequence set forth in SEQ ID NO:1. In some embodiments, the light-responsive cation channel comprises a L132C substitution of the amino acid sequence set forth in SEQ ID NO:1. In some embodiments, the light-responsive cation channel comprises an E123T substitution of the amino acid sequence set forth in SEQ ID NO:1. In some embodiments, the light-responsive cation channel comprises an E123A substitution of the amino acid sequence set forth in SEQ ID NO:1. In some embodiments, the light-responsive cation channel comprises a T159C substitution and an E123T substitution of the amino acid sequence set forth in SEQ ID NO:1. In some embodiments, the light-responsive cation channel comprises a T159C substitution and an E123A substitution of the amino acid sequence set forth in SEQ ID NO:1. In some embodiments, the light-responsive cation channel comprises a T159C substitution, an L132C substitution, and an E123T substitution of the amino acid sequence set forth in SEQ ID NO:1. In some embodiments, the light-responsive cation channel comprises a T159C substitution, an L132C substitution, and an E123A substitution of the amino acid sequence set forth in SEQ ID NO:1. In some embodiments, the light-responsive cation channel comprises an L132C substitution and an E123T substitution of the amino acid sequence set forth in SEQ ID NO:1. In some embodiments, the light-responsive cation channel comprises an L132C substitution and an E123A substitution of the amino acid sequence set forth in SEQ ID NO:1.

Further disclosure related to light-responsive cation channel proteins can be found in U.S. Patent Application Publication No. 2007/0054319 and International Patent Application Publication Nos. WO 2009/131837 and WO 2007/024391, the disclosures of each of which are hereby incorporated by reference in their entireties.

### Step Function Opsins and Stabilized Step Function Opsins

In other embodiments, the light-responsive cation channel protein can be a step function opsin (SFO) protein or a stabilized step function opsin (SSFO) protein that can have specific amino acid substitutions at key positions throughout the retinal binding pocket of the protein. In some embodiments, the SFO protein can have a mutation at amino acid residue C128 of SEQ ID N0:1. In other embodiments, the SFO protein has a C128A mutation in SEQ ID NO:1. In other embodiments, the SFO protein has a C128S mutation in SEQ ID NO:1. In another embodiment, the SFO protein has a C128T mutation in SEQ ID NO:1. In some embodiments, the SFO protein can comprise an amino acid sequence at least about 90%, 91%, 92%, 93%, 94%, 95%, 96%, 97%, 98%, 99%, or 100% identical to the sequence shown in SEQ ID NO:2, SEQ ID NO:3, or SEQ ID NO:4.

In some embodiments, the SFO protein can have a mutation at amino acid residue D156 of SEQ ID NO:1. In some embodiments, the SFO protein can comprise an amino acid sequence at least about 90%, 91%, 92%, 93%, 94%, 95%, 96%, 97%, 98%, 99%, or 100% identical to the sequence shown in SEQ ID NO:5.

In other embodiments, the SSFO protein can have a mutation at both amino acid residues C128 and D156 of SEQ ID NO:1. In one embodiment, the SSFO protein has an C128S and a D156A mutation in SEQ ID NO:1. In another embodiment, the SSFO protein can comprise an amino acid sequence at least about 90%, 91%, 92%, 93%, 94%, 95%, 96%, 97%, 98%, 99%, or 100% identical to the sequence shown in SEQ ID NO:6. In another embodiment, the SSFO protein can comprise a C128T mutation in SEQ ID NO:1. In some embodiments, the SSFO protein comprises C128T and D156A mutations in SEQ ID NO:1.

In some embodiments the SFO or SSFO proteins provided herein can be capable of mediating a depolarizing current in the cell when the cell is illuminated with blue light. In other embodiments, the light can have a wavelength of about 445 nm. Additionally, the light can have an intensity of about 100 Hz. In some embodiments, activation of the SFO or SSFO protein with light having an intensity of 100 Hz can cause depolarization-induced synaptic depletion of the neurons expressing the SFO or SSFO protein. In some embodiments, each of the disclosed step function opsin and stabilized step function opsin proteins can have specific properties and characteristics for use in depolarizing the membrane of a neuronal cell in response to light.

Further disclosure related to SFO or SSFO proteins can be found in International Patent Application Publication No. WO 2010/056970 and U.S. Provisional Patent Application Nos. 61/410,704 and 61/511,905, the disclosures of each of which are hereby incorporated by reference in their entireties.

### C1V1 Chimeric Cation Channels

In other embodiments, the light-responsive cation channel protein can be a C1V1 chimeric protein derived from the VChR1 protein of Volvox carteri and the ChR1 protein from Chlamydomonas reinhardti, wherein the protein comprises the amino acid sequence of VChR1 having at least the first and second transmembrane helices replaced by the first and second transmembrane helices of ChR1; is responsive to light; and is capable of mediating a depolarizing current in the cell when the cell is illuminated with light. In some embodiments, the C1V1 protein can further comprise a replacement within the intracellular loop domain located between the second and third transmembrane helices of the chimeric light responsive protein, wherein at least a portion of the intracellular loop domain is replaced by the corresponding portion from ChR1. In another embodiment, the portion of the intracellular loop domain of the C1V1 chimeric protein can be replaced with the corresponding portion from ChR1 extending to amino acid residue A145 of the ChR1. In other embodiments, the C1V1 chimeric protein can further comprise a replacement within the third transmembrane helix of the chimeric light responsive protein, wherein at least a portion of the third transmembrane helix is replaced by the corresponding sequence of ChR1. In yet another embodiment, the portion of the intracellular loop domain of the C1V1 chimeric protein can be replaced with the corresponding portion from ChR1 extending to amino acid residue W163 of the ChR1. In other embodiments, the C1V1 chimeric protein can comprise an amino acid sequence at least about 90%, 91%, 92%, 93%, 94%, 95%, 96%, 97%, 98%, 99%, or 100% identical to the sequence shown in SEQ ID NO:13 or SEQ ID NO:49.

In some embodiments, the C1V1 protein can mediate a depolarizing current in the cell when the cell is illuminated with green light. In other embodiments, the light can have a wavelength of between about 540 nm to about 560 nm. In some embodiments, the light can have a wavelength of about 542 nm. In some embodiments, the C1V1 chimeric protein is not capable of mediating a depolarizing current in the cell when the cell is illuminated with violet light. In some embodiments, the chimeric protein is not capable of mediating a depolarizing current in the cell when the cell is illuminated with light having a wavelength of about 405 nm. Additionally, the light can have an intensity of about 100 Hz. In some embodiments, activation of the C1V1 chimeric protein with light having an intensity of 100 Hz can cause depolarization-induced synaptic depletion of the neurons expressing the C1V1 chimeric protein. In some embodiments, the disclosed C1V1 chimeric protein can have specific properties and characteristics for use in depolarizing the membrane of a neuronal cell in response to light.

### C1V1 Chimeric Mutant Variants

In some aspects, the present disclosure provides polypeptides comprising substituted or mutated amino acid sequences, wherein the mutant polypeptide retains the characteristic light-activatable nature of the precursor C1V1 chimeric polypeptide but may also possess altered properties in some specific aspects. For example, the mutant light-responsive C1V1 chimeric proteins described herein can exhibit an increased level of expression both within an animal cell or on the animal cell plasma membrane; an altered responsiveness when exposed to different wavelengths of light, particularly red light; and/or a combination of traits whereby the chimeric C1V1 polypeptide possess the properties of low desensitization, fast deactivation, low violet-light activation for minimal cross-activation with other light-responsive cation channels, and/or strong expression in animal cells.

Accordingly, provided herein are C1V1 chimeric light-responsive opsin proteins that can have specific amino acid substitutions at key positions throughout the retinal binding pocket of the VChR1 portion of the chimeric polypeptide. In some embodiments, the C1V1 protein can have a mutation at amino acid residue E122 of SEQ ID NO:13 or SEQ ID NO:49. In some embodiments, the C1V1 protein can have a mutation at amino acid residue E162 of SEQ ID NO:13 or SEQ ID NO:49. In other embodiments, the C1V1 protein can have a mutation at both amino acid residues E162 and E122 of SEQ ID NO:13 or SEQ ID NO:49. In other embodiments, the C1V1 protein can comprise an amino acid sequence at least about 90%, 91%, 92%, 93%, 94%, 95%, 96%, 97%, 98%, 99%, or 100% identical to the sequence shown in SEQ ID NO:14, SEQ ID NO:15, SEQ ID NO:16, SEQ ID NO:17, SEQ ID NO:18, or SEQ ID NO:19. In some embodiments, each of the disclosed mutant C1V1 chimeric proteins can have specific properties and characteristics for use in depolarizing the membrane of an animal cell in response to light.

In some aspects, the C1V1-E122 mutant chimeric protein is capable of mediating a depolarizing current in the cell when the cell is illuminated with light. In some embodiments the light can be green light. In other embodiments, the light can have a wavelength of between about 540 nm to about 560 nm. In some embodiments, the light can have a wavelength of about 546 nm. In other embodiments, the C1V1-E122 mutant chimeric protein can mediate a depolarizing current in the cell when the cell is illuminated with red light. In some embodiments, the red light can have a wavelength of about 630 nm. In some embodiments, the C1V1-E122 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. Additionally, the light can have an intensity of about 100 Hz. In some embodiments, activation of the C1V1-E122 mutant chimeric protein with light having an intensity of 100 Hz can cause depolarization-induced synaptic depletion of the neurons expressing the C1V1-E122 mutant chimeric protein. In some embodiments, the disclosed C1V1-E122 mutant chimeric protein can have specific properties and characteristics for use in depolarizing the membrane of a neuronal cell in response to light.

In other aspects, the C1V1-E162 mutant chimeric protein is capable of mediating a depolarizing current in the cell when the cell is illuminated with light. In some embodiments the light can be green light. In other embodiments, the light can have a wavelength of between about 535 nm to about 540 nm. In some embodiments, the light can have a wavelength of about 542 nm. In other embodiments, the light can have a wavelength of about 530 nm. In some embodiments, the C1V1-E162 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. Additionally, the light can have an intensity of about 100 Hz. In some embodiments, activation of the C1V1-E162 mutant chimeric protein with light having an intensity of 100 Hz can cause depolarization-induced synaptic depletion of the neurons expressing the C1V1-E162 mutant chimeric protein. In some embodiments, the disclosed C1V1-E162 mutant chimeric protein can have specific properties and characteristics for use in depolarizing the membrane of a neuronal cell in response to light.

In yet other aspects, the C1V1-E122/E162 mutant chimeric protein is capable of mediating a depolarizing current in the cell when the cell is illuminated with light. In some embodiments the light can be green light. In other embodiments, the light can have a wavelength of between about 540 nm to about 560 nm. In some embodiments, the light can have a wavelength of about 546 nm. In some embodiments, the C1V1-E122/E162 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. In some embodiments, the C1V1-E122/E162 mutant chimeric protein can exhibit less activation when exposed to violet light relative to C1V1 chimeric proteins lacking mutations at E122/E162 or relative to other light-responsive cation channel proteins. Additionally, the light can have an intensity of about 100 Hz. In some embodiments, activation of the C1V1-E122/E162 mutant chimeric protein with light having an intensity of 100 Hz can cause depolarization-induced synaptic depletion of the neurons expressing the C1V1-E122/E162 mutant chimeric protein. In some embodiments, the disclosed C1V1-E122/E162 mutant chimeric protein can have specific properties and characteristics for use in depolarizing the membrane of a neuronal cell in response to light.

Further disclosure related to C1V1 chimeric cation channels as well as mutant variants of the same can be found in U.S. Provisional Patent Application Nos. 61/410,736, 61/410,744, and 61/511,912, the disclosures of each of which are hereby incorporated by reference in their entireties.

### Champ

In some embodiments, the light-responsive protein is a chimeric protein comprising Arch-TS-p2A-ASIC 2a-TS-EYFP-ER-2 (Champ). Champ comprises an Arch domain and an Acid-sensing ion channel (ASIC)-2a domain. Light activation of Champ activates a proton pump (Arch domain) that activates the ASIC-2a proton-activated cation channel (ASIC-2a domain). A polynucleotide encoding Champ is shown in SEQ ID NO:50.

### Polynucleotides

The disclosure also provides polynucleotides comprising a nucleotide sequence encoding a light-responsive protein described herein. In some embodiments, the polynucleotide comprises an expression cassette. In some embodiments, the polynucleotide is a vector comprising the above-described nucleic acid. In some embodiments, the nucleic acid encoding a light-responsive protein of the disclosure is operably linked to a promoter. Promoters are well known in the art. Any promoter that functions in the host cell can be used for expression of the light-responsive opsin proteins and/or any variant thereof of the present disclosure. In one embodiment, the promoter used to drive expression of the light-responsive opsin proteins can be a promoter that is specific to motor neurons. In other embodiments, the promoter is capable of driving expression of the light-responsive opsin proteins in neurons of both the sympathetic and/or the parasympathetic nervous systems. Initiation control regions or promoters, which are useful to drive expression of the light-responsive opsin proteins or variant thereof in a specific animal cell are numerous and familiar to those skilled in the art. Virtually any promoter capable of driving these nucleic acids can be used. Examples of motor neuron-specific genes can be found, for example, in Kudo, et al., Human Mol. Genetics, 2010, 19(16): 3233-3253, the contents of which are hereby incorporated by reference in their entirety. In some embodiments, the promoter used to drive expression of the light-responsive protein can be the Thy1 promoter, which is capable of driving robust expression of transgenes in neurons of both the central and peripheral nervous systems (See, e.g., Llewellyn, et al., 2010, Nat. Med., 16(10):1161-1166). In other embodiments, the promoter used to drive expression of the light-responsive protein can be the EF1.alpha. promoter, a cytomegalovirus (CMV) promoter, the CAG promoter, a synapsin-I promoter (e.g., a human synapsin-I promoter), a human synuclein 1 promoter, a human Thy1 promoter, a calcium/calmodulin-dependent kinase II alpha (CAMKII.alpha.) promoter, or any other promoter capable of driving expression of the light-responsive opsin proteins in the peripheral neurons of mammals.

Also provided herein are vectors comprising a nucleotide sequence encoding a light-responsive protein or any variant thereof described herein. The vectors that can be administered according to the present invention also include vectors comprising a nucleotide sequence which encodes an RNA (e.g., an mRNA) that when transcribed from the polynucleotides of the vector will result in the accumulation of light-responsive opsin proteins on the plasma membranes of target animal cells. Vectors which may be used, include, without limitation, lentiviral, HSV, adenoviral, and adeno-associated viral (AAV) vectors. Lentiviruses include, but are not limited to HIV-1, HIV-2, SIV, FIV and EIAV. Lentiviruses may be pseudotyped with the envelope proteins of other viruses, including, but not limited to VSV, rabies, Mo-MLV, baculovirus and Ebola. Such vectors may be prepared using standard methods in the art.

In some embodiments, the vector is a recombinant AAV vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p15-23, Budder Arnold, London, UK (2006), the disclosures of each of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Methods of preparing AAV vectors in a baculovirus system are described in, e.g., WO 2008/024998. AAV vectors can be self-complementary or single-stranded. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos.: 91/18088 and WO 93/09239; U.S. Pat. Nos. 4,797,368, 6,596,535, and 5,139,941; and European Patent No.: 0488528, all of which are hereby incorporated by reference herein in their entireties). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the present disclosure can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the present disclosure are encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the present disclosure includes a recombinant virus particle (recombinant because it contains a recombinant polynucleotide) comprising any of the vectors described herein. Methods of producing such particles are known in the art and are described in U.S. Pat. No. 6,596,535, the disclosure of which is hereby incorporated by reference in its entirety.

The light sensitive protein may be delivered to the target tissue using a virus. The virus may be selected from the group consisting of: AAV1, AAV2, AAV4, AAV5, AAV6, AAV7, AAV8, AAV9, lentivirus, and HSV. The virus may contain a polynucleotide that encodes for the opsin protein. The polynucleotide may encode for a transcription promoter. The transcription promoter may be selected from the group consisting of: CaMKIIa, hSyn, CMV, Hb9Hb, Thy1, and Ef1a. The viral construct may be selected from the group consisting of: AAV1-hSyn-Arch3.0, AAV5-CamKII-Arch3.0, AAV1-hSyn-iC1C23.0, AAV5-CamKII-iC1C23.0, AAV1-hSyn-SwiChR3.0, and AAV5-CamKII-SwiChR3.0. Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed:

1. A probe for illuminating a target tissue of a patient, comprising:
a. a plurality of optical fibers;
b. a probe body portion having proximal and distal ends, the probe body portion being moveably coupled to the plurality of fibers and configured to at least partially encapsulate the plurality of fibers;
c. a distal end portion coupled to the distal end of the probe body portion, the distal end portion comprising at least one guiding feature configured to redirect a path of at least one of the optical fibers comprising the plurality of optical fibers as such at least portion of one of the optical fibers is extended through and past the distal end portion by moving the plurality of fibers relative to the probe body portion.

2. The probe of claim 1, further comprising an ejector portion configured to move the plurality of fibers relative to the probe body portion.

3. The probe of claim 2, wherein the ejector portion comprises an elongate member configured to advance the plurality of fibers relative to the probe body portion, the elongate portion coupled to the plurality of fibers.

4. The probe of claim 3, wherein the elongate member comprises an elongate structure selected from the group consisting of: a wire, a fiber, a rod, and a tube.

5. The probe of claim 3, wherein the elongate member comprises a polymer or metal.

6. The probe of claim 3, further comprising a collar member coupled to both the plurality of fibers and the elongate member.

7. The probe of claim 2, wherein the ejector portion comprises a collectively grouped portion of the plurality of fibers, and wherein the at least a portion one of the optical fibers is extended through and past the distal end portion by moving the collectively grouped portion relative to the probe body portion.

8. The probe of claim 1, wherein at least one of the plurality of optical fibers comprises glass or polymer.

9. The probe of claim 1, wherein the probe body portion comprises an at least partially circumferentially coupled member relative to the plurality of optical fibers.

10. The probe of claim 9, wherein the probe body portion comprises a structure selected from the group consisting of: a tube, coil, or spring.

11. The probe of claim 10, wherein the probe body portion comprises a tube having one or more relief cuts formed in it to increase overall structural flexibility of the tube.

12. The probe of claim 11, wherein the one or more relief cuts are formed in an interrupted helical pattern.

13. The probe of claim 1, wherein the probe body portion comprises a polymer or metal material.

14. The probe of claim 1, wherein the probe body portion comprises a material selected to have a relatively low friction coefficient relative to the plurality of optical fibers.

15. The probe of claim 14, wherein the probe body portion comprises a hydrophilic coating configured to provide relatively low friction resistance to the plurality of optical fibers when in a fluid-exposed environment.

16. The probe of claim 1, wherein at least one of the plurality of optical fibers comprises a pre-set shape, such that when extended through and past the distal end portion, the at least one of the plurality of optical fibers is biased to occupy such pre-set shape.

17. The probe of claim 1, wherein the plurality of optical fibers comprises fibers of varying lengths, such that upon extension through and past the distal end portion, they form a non-symmetric pattern.

18. The probe of claim 1, wherein the plurality of optical fibers is configured to inserted into brain or spinal cord tissue structures.

19. The probe of claim 1, further comprising an infusion cannula bundled with the plurality of optical fibers, the infusion cannula having proximal and distal ends and defining a lumen therebetween.

20. The probe of claim 19, wherein the lumen is configured to facilitate infusion of liquid compounds from the proximal end, wherein a medical provider may have direct access, to the distal end adjacent the target tissue of the patient.

21. The probe of claim 20, wherein the lumen may be configured to facilitate delivery of liquid compounds comprising genetic material.

22. The probe of claim 1, wherein the plurality of optical fibers is configured to transmit wavelengths in the range of about 400 nm to about 700 nm.

23. The probe of claim 21, wherein the liquid compounds comprise optogenetic material.

24. An optical diffuser, comprising:
a composite comprising a generally cylindrical outer shape and configured to emit light along its length through its outer surface;
wherein the composite comprises a matrix material and a plurality of scattering particles embedded in the matrix material, the plurality of scattering particles having a refractive index that is different from the refractive index of the matrix material.

25. The optical diffuser of claim 1, further comprising an interface configured to provide for direct coupling between the diffuser and an optical fiber.

26. The optical diffuser of claim 1, wherein the scattering particles comprise microspheres.

27. The optical diffuser of claim 1, wherein the scattering particles comprise a material selected from the group consisting of: polytetrafluoroethylene (PTFE), polycarbonate (PC), polystyrene (PS), silicon dioxide (SiO2), borosilicate glass, dense flint glass, soda lime glass, barium sulfate (BaSO4), titanium dioxide (TiO2), and aluminum oxide (Al2O3).

28. The optical diffuser of claim 27, wherein the scattering particles comprise borosilicate glass sold under the tradename BK7.

29. The optical diffuser of claim 27, wherein the scattering particles comprise dense flint glass sold under the tradename SF10.

30. The optical diffuser of claim 24, wherein the matrix material is selected from the group consisting of: a polymer, a gel, an epoxy, a heat-cured material, and a light-cured material.

31. The optical diffuser of claim 24, wherein the scattering particles occupy a volume fraction of between about 0.1% and about 10% within the composite.

32. The optical diffuser of claim 24, wherein the scattering particles have a characteristic size of between about 0.10 microns and about 10 microns.

33. The optical diffuser of claim 24, wherein the scattering particles have a refractive index that is greater than the refractive index of the matrix material.

34. The optical diffuser of claim 24, wherein the scattering particles have a refractive index that is less than the refractive index of the matrix material.

35. The optical diffuser of claim 24, further comprising a sheath configured to at least partially encapsulate the composite.

36. The optical diffuser of claim 35, wherein the sheath is coupled to the composite using an adhesive.

37. The optical diffuser of claim 36, wherein the adhesive has a refractive index that is less than the refractive index of the matrix material.

38. The optical diffuser of claim 35, wherein the sheath comprises a material selected from the group consisting of: polyethylene (PE), polyethylene terephthalate (PET), polyethylene terephthalate glycol (PETG), and polytetrafluoroethylene (PTFE).

39. An optical diffuser, comprising:
an optical waveguide featuring a plurality of cuts configured to emit light along the length of the waveguide through an outer surface of the waveguide.

40. The optical diffuser of claim 39, wherein the plurality of cuts are oriented at an angle nominally perpendicular to the surface of the waveguide.

41. The optical diffuser of claim 39, wherein the plurality of cuts are oriented at an angle nominally oblique to the surface of the waveguide.

42. The optical diffuser of claim 39, wherein an orientation angle of one or more of the plurality of cuts may be specifically configured to cause asymmetric diffusion of light out of the diffuser from the waveguide.

43. The optical diffuser of claim 42, wherein the orientation angle of the plurality of cuts is varied in a pattern to cause asymmetric diffusion of light out of the diffuser from the waveguide.

44. The optical diffuser of claim 43, wherein the orientation angle of the plurality of cuts is varied in a longitudinal pattern along the waveguide to cause asymmetric diffusion of light out of the diffuser from the waveguide.

45. The optical diffuser of claim 43, wherein the orientation angle of the plurality of cuts is varied in a pattern of discrete zones to create discrete diffuser segments.

46. The optical diffuser of claim 39, wherein a depth of one or more of the plurality of cuts may be specifically configured to cause asymmetric diffusion of light out of the diffuser from the waveguide.

47. The optical diffuser of claim 46, wherein the depth of the plurality of cuts is varied in a pattern to cause asymmetric diffusion of light out of the diffuser from the waveguide.

48. The optical diffuser of claim 47, wherein the depth of the plurality of cuts is varied in a longitudinal pattern along the waveguide to cause asymmetric diffusion of light out of the diffuser from the waveguide.

49. The optical diffuser of claim 47, wherein the orientation angle of the plurality of cuts is varied in a pattern of discrete zones to create discrete diffuser segments.

50. An optical connection assembly, comprising:
a. a first faceplate comprising a plurality of first fiber ports configured to provide direct contact with faces of first optical fibers coupled thereto, wherein the first fiber ports are arranged in a predetermined first two-dimensional pattern;
b. a second faceplate comprising a plurality of second fiber ports configured to provide direct contact with faces of second optical fibers coupled thereto, wherein the second fiber ports are arranged in a predetermined second two-dimensional pattern, the second two-dimensional pattern complementary with the first two-dimensional pattern;
c. an alignment portion configured to orient the first faceplate with the second faceplate such that the first and second two-dimensional patterns are substantially aligned; and
d. a locking portion configured to secure a coupling between the first faceplate and second faceplate.

51. The optical connection assembly of claim 50, wherein the first two-dimensional pattern is a regular array.

52. The optical connection assembly of claim 51, wherein the regular array is a hexagonal array.

53. The optical connection assembly of claim 50, wherein the first fiber ports are configured to be of different size than the second fiber ports.

54. The optical connection assembly of claim 53, wherein the first fiber ports are configured to be smaller than the second fiber ports.

55. The optical connection assembly of claim 50, wherein the alignment portion comprises a non-radially symmetric tongue-in-groove configuration.

56. The optical connection assembly of claim 50, wherein the locking portion comprises at least one set of complementary interlocking teeth.

57. The optical connection assembly of claim 50, wherein the locking portion and alignment portion are integrated into a common member.

58. The optical connection assembly of claim 57, wherein the locking portion and alignment portion comprise a non-radially symmetric tongue-in-groove configuration with at least one set of complementary interlocking teeth.

59. An anchoring assembly for coupling a portion of a probe to the cranium of a patient, comprising:
a. a ring portion comprising one or more mounting tabs, a channel, and having an inner and outer diameter, the ring portion being configured to be permanently positioned at least partially within a hole created through the cranium of the patient, wherein the one or more mounting tabs are arranged about the outer diameter of the ring portion and configured to be positioned against an exterior surface of the cranium, and wherein the channel is configured to accommodate passage of at least a portion of the probe;
b. a collet portion having an outer diameter and inner diameter, and defining a channel and a slot, the outer diameter being selected to engage with the inner diameter of the ring portion, wherein the channel is configured to be complementary to the channel of the ring portion and also configured to accommodate passage of at least a portion of the probe, and wherein the slot is located opposite the channel and configured to accommodate passage of at least a portion of the probe; and
c. a cap portion comprising a cap slot that is complementary to the slot of the collet portion and sized to fit within the inner diameter of the collet portion;
wherein the ring channel, collet channel, and cap slot are configured to avoid kinking the at least a portion of the probe as the it is passed, at least in part, across a wall of the cranium.

60. The anchoring assembly of claim 59, wherein the one or more mounting tabs are configured to be positioned in a location selected from those consisting of: near a bottom of the ring portion; near a top of the ring portion; and in between a bottom and a top of the ring portion.

61. The anchoring assembly of claim 59, further comprising a snap-fit feature formed within the inner diameter of the ring portion, the snap-fit feature configured to mate with the outer diameter of the collet portion.

62. The anchoring assembly of claim 59, further comprising a snap-fit feature formed within the inner diameter of the collet portion, the snap-fit feature configured to mate with the outer diameter of the cap portion.

63. The anchoring assembly of claim 59, wherein the ring portion comprises a metal or polymer.

64. The anchoring assembly of claim 59, wherein the collet portion comprises a metal or polymer.

65. The anchoring assembly of claim 59, wherein the cap portion comprises a metal or polymer.

66. The anchoring assembly of claim 59, wherein the ring channel, collet channel, and cap slot are configured to maintain a minimum bend radius of the at least a portion of the probe.

67. The anchoring assembly of claim 66, wherein the minimum bend radius of the at least a portion of the probe is greater than or equal to 3.5 mm.

68. A therapeutic system for illuminating tissue, comprising:
a. a power supply;
b. a controller;
c. an illumination source operatively coupled to the controller and power supply;
d. an applicator operatively coupled to the illumination source and also configured to engage a targeted tissue structure, the applicator configured to receive photons from the illumination source and deliver at least a portion of the received photons into the targeted tissue structure;
wherein the controller is configured to control the illumination source to emit photons to the targeted tissue structure with an illumination configuration selected to avoid phototoxicity of the targeted tissue structure with prolonged use.

69. The therapeutic system of claim 68, wherein the illumination configuration comprises a pulsatile emission configuration configured to provide a fluence rate of less than about 55 milliwatts per square millimeter.

70. The therapeutic system of claim 68, wherein the illumination configuration comprises a pulsatile emission configuration configured to provide a fluence rate of greater than 55 milliwatts per square millimeter only in a volume immediately adjacent to the applicator, and less than about 55 milliwatts per square millimeter elsewhere.

71. The therapeutic system of claim 69, wherein the pulsatile emission configuration has a duty cycle of less than or equal to about 20%.

72. The therapeutic system of claim 70, wherein the pulsatile emission configuration has a duty cycle of less than or equal to about 20%.

73. The therapeutic system of claim 69, wherein the pulsatile emission configuration has a pulse off time of greater than or equal to about 50 milliseconds.

74. The therapeutic system of claim 70, wherein the pulsatile emission configuration has a pulse off time of greater than or equal to about 50 milliseconds.

75. The therapeutic system of claim 69, wherein the pulsatile emission configuration has a pulse on time of less than or equal to about 20 milliseconds.

76. The therapeutic system of claim 70, wherein the pulsatile emission configuration has a pulse on time of less than or equal to about 20 milliseconds.

77. The therapeutic system of claim 70, wherein the volume immediately adjacent to the applicator comprises a thickness of less than or equal to about 300 microns.

78. The therapeutic system of claim 68, wherein the illumination configuration comprises a continuous emission configuration configured to provide a fluence rate of less than about 2.5 milliwatts per square millimeter.

79. The therapeutic system of claim 68, wherein the illumination configuration comprises a continuous emission configuration configured to provide a fluence rate of greater than 2.5 milliwatts per square millimeter only in a volume immediately adjacent to the applicator, and less than about 2.5 milliwatts per square millimeter elsewhere.

80. The therapeutic system of claim 79, wherein the volume immediately adjacent to the applicator comprises a thickness of less than or equal to about 300 microns.

81. The therapeutic system of claim 68, wherein the applicator is an implantable applicator.

82. The therapeutic system of claim 68, wherein the implantable applicator is configured to be engaged with a targeted tissue structure that comprises a portion of the nervous system.

83. The therapeutic system of claim 82, wherein the implantable applicator is configured to be engaged with a targeted tissue structure that comprises a nerve or a portion of the central nervous system.

84. The system of claim 82, wherein the targeted tissue structure has been genetically modified to encode an opsin protein.

85. The system of claim 84, wherein the opsin protein is an inhibitory opsin protein.

86. The system of claim 85, wherein the inhibitory opsin protein is selected from the group consisting of: NpHR, eNpHR 1.0, eNpHR 2.0, eNpHR 3.0, SwiChR, SwiChR 2.0, SwiChR 3.0, Arch, ArchT, Arch 3.0, ArchT 3.0, iChR, iC++, ChloC, Slow ChloC, iC1C2, iC1C2 2.0, and iC1C2 3.0.

87. The system of claim 85, wherein the opsin protein is a stimulatory opsin protein.

88. The system of claim 86, wherein the stimulatory opsin protein is selected from the group consisting of: ChR2, C1V1-T, C1V1-TT, Chronos, Chrimson, ChrimsonR, CatCh, VChR1-SFO, ChR2-SFO, ChR2-SSFO, ChEF, ChIEF, and Jaws.
